(19) United States
(12) Patent Application Publication
(10) Pub. No.: US 2024/0021280 A1
(43) Pub. Date: Jan. 18, 2024

(54) ONCOLOGY WORKFLOW FOR CLINICAL DECISION SUPPORT

(71) Applicant: Roche Molecular Systems, Inc., Pleasanton, CA (US)

(72) Inventors: Cindy K. BARNARD, Pleasanton, CA (US); Sambasivarao BYRAPUNENI, Pleasanton, CA (US); Diwakar CHAPAGAIN, Pleasanton, CA (US); Archana P. DORGE, Pleasanton, CA (US); Catherine M. JEU, Pleasanton, CA (US); Rengaraja KESAVAN, Pleasanton, CA (US); Kaushal D. PAREKH, Pleasanton, CA (US); Raman RAMANATHAN, Pleasanton, CA (US); David M. SCHLOSSMAN, Pleasanton, CA (US); Vishakha SHARMA, Pleasanton, CA (US)

(21) Appl. No.: 18/222,308

(22) Filed: Jul. 14, 2023

Related U.S. Application Data
(63) Continuation of application No. PCT/US22/12814, filed on Jan. 18, 2022.
(60) Provisional application No. 63/138,275, filed on Jan. 15, 2021.
(60) Provisional application No. 63/256,476, filed on Oct. 15, 2021.

(51) Int. Cl. G16H 10/60; G16H 15/00; G16H 50/20

(52) U.S. Cl. CPC G16H 10/60; G16H 15/00; G16H 50/20

(57) ABSTRACT

Systems and methods are provided for managing patient data. The system integrates medical data from multiple sources to a unified patient database. Structured and unstructured medical data is obtained, enriched (e.g., by designating data field types, standardizing data types or terminology, and the like), and stored to the unified patient database. The data retrieved from the disparate sources is stored to data elements in the unified patient database in a network of connected objects including data about tumor masses, treatments, reports, medical history, and diagnoses. The data in the unified patient database is used to display patient data in user-friendly interface views, including a patient journey view that displays patient data in a chronological fashion organized by data types. The different interface views can be traversed to display patient data originating from disparate sources with ease, to improve the clinical decision making process.

### CROSS-REFERENCE TO RELATED PATENT APPLICATIONS

The present application is a bypass continuation of International Application No. PCT/US2022/012814 filed Jan. 18, 2022, which claims benefit of priority to U.S. Patent Application No. 63/138,275, filed Jan. 15, 2021 and U.S. Patent Application No. 63/256,476, filed Oct. 15, 2021, each of which is incorporated herein by reference for all purposes.

### BACKGROUND

Every day, hospitals create a tremendous amount of clinical data across the globe. Analysis of this data is critical to understand detailed insights in healthcare delivery and quality of care, as well as provide a basis to improve personalized healthcare. Unfortunately, a large proportion of recorded data is difficult to access and analyze as most data are captured in an unstructured form. Unstructured data may include, for examples, healthcare provider notes, imaging or pathology reports, or any other data that are neither associated with a structured data model nor organized in a pre-defined manner to define the context and/or meaning of the data. The data are typically stored in multiple data sources. A clinician who seeks to analyze the data of a patient to make a decision may need to source the data from multiple data sources, and then parse through the data manually to extract the information needed to make a clinical decision. But such a way of obtaining data to make a clinical decision is laborious, slow, costly, and error-prone.

### BRIEF SUMMARY

Disclosed herein are techniques for improving a clinician's access to patient data to perform a clinical decision, such as a clinical decision related to oncology. In some examples, a medical data processing system is provided. The medical data processing system can collect medical data of a patient from multiple data sources, convert the medical data into structured data, and present the structured data in various forms, such as in a summary format, and in a longitudinal temporal view report format. The medical data processing system can also support an oncology workflow solution, which can support or perform a diagnosis operation on the collected medical data, and present a result of the diagnosis to the clinician. The oncology workflow solution can enable a clinician, such as an oncologist or his/her delegates, to longitudinally manage cancer patients from suspicion of cancer through treatment and follow-up. The oncology workflow solution can also support other medical applications, such as a quality of care evaluation tool to evaluate a quality of care administered to a patient, a medical research tool to determine a correlation between various information of the patient (e.g., demographic information) and tumor information (e.g., prognosis or expected survival) of the patient, etc. The techniques can also be applied to other types of diseases areas and not limited to oncology.

In some embodiments, a method for managing medical data includes performing by a server computer: creating a patient record for a patient in a unified patient database, the patient record comprising an identifier of the patient and one or more data objects related to medical data associated with the patient, the unified patient database including data from a plurality of sources; retrieving, from an external database, a medical record for the patient; receiving identification of a primary cancer associated with the medical record via a Graphical User Interface (GUI); in response to receiving the identification of the primary cancer, creating a primary cancer object in the patient record, the primary cancer object having a field including the primary cancer; storing the medical record linked to the primary cancer object in the patient record in the unified patient database; receiving, via user input to the GUI, medical data for the patient; determining that the medical data for the patient is associated with the primary cancer; and storing the medical data for the patient linked to the primary cancer object in the patient record in the unified patient database.

In some aspects, the medical record for the patient is in a first format comprising a set of data elements correlated to corresponding data types; and receiving the identification of the primary cancer comprises: identifying the primary cancer by analyzing the data elements and the data types; displaying the GUI comprising a prompt for a user to confirm the primary cancer identification; and receiving user confirmation of the primary cancer identification via the GUI.

In some aspects, the medical record is a first medical record, the method further comprising: receiving a second medical record for the patient, wherein the second medical record is in a second format comprising unstructured data; identifying, from the unstructured data, a data element associated with the primary cancer; analyzing the unstructured data to assign the data element to a data type; and based on the assigned data type and the identifying the data element is associated with the primary cancer, storing the data element linked to the primary cancer object in the patient record in the unified patient database.

In some aspects, receiving the identification of the primary cancer associated with the medical record comprises: displaying, via the GUI, the medical record and a menu configured to receive user input selecting one or more primary cancers; and receiving, via the GUI, user input selecting the primary cancer.

In some aspects, the method further comprises storing the medical record in the patient record; and parsing the medical record to determine that the patient record is not associated with a particular primary cancer, wherein displaying the medical record and the menu is responsive to determining that the patient record is not associated with a particular primary cancer.

In some aspects, the medical record comprises unstructured data; and the method further comprises: applying a first machine learning model to identify text in the medical record; and applying a second machine learning model to correlate a portion of the identified text with a corresponding field, wherein storing the medical record further comprises storing the identified text to the unified patient database in association with the field. In some aspects, the first machine learning model comprises an Optical Character Recognition (OCR) model; and the second machine learning model comprises a Natural Language Processing (NLP) model.

In some aspects, the method further comprises retrieving, from the unified patient database, at least a subset of the medical data for the patient; and causing display, via a user interface, of the at least the subset of the medical data for the patient for performing clinical decision making. In some aspects, the external database corresponds to at least one of: an EMR (electronic medical record) system, a PACS (picture archiving and communication system), a Digital Pathology (DP) system, an LIS (laboratory information system), and a RIS (radiology information system). In some aspects, the medical record is retrieved based upon the identifier of the patient.

In some embodiments, a method for managing a unified patient database comprising performing by a server computer: storing, to the unified patient database, a patient record comprising a network of interconnected data objects, the unified patient database including data from a plurality of sources; storing, to the patient record in the unified patient database, a first data object corresponding to a data element for a tumor mass of a primary cancer, the first data object including an attribute specifying a site of the tumor mass; receiving, from a diagnostic computer, diagnosis information corresponding to the primary cancer; analyzing the diagnosis information to identify a correlation between the diagnosis information and to the tumor mass; based on identifying the correlation between the diagnosis information and the tumor mass, storing, to the unified patient database, a second data object corresponding to the diagnosis information, the second data object connected to the first data object via the network of interconnected data objects; receiving, from the diagnostic computer, treatment information corresponding to the primary cancer; analyzing the treatment information to identify a correlation between the treatment information and to the tumor mass; and based on identifying the correlation between the treatment information and the tumor mass, storing, to the unified patient database, a third data object corresponding to the treatment information, the third data object connected to the first data object via the network of interconnected data objects.

In some aspects, the method further comprises retrieving, from the unified patient database, one or more of the attributes specifying the site of the tumor mass, the diagnosis information, and/or the treatment information; and causing display, via a user interface, of one or more of the attribute specifying the site of the tumor mass, the diagnosis information, and/or the treatment information for clinical decision making.

In some aspects, the method further comprises receiving, from the diagnostic computer, patient history data; analyzing the patient history data to identify a correlation between the patient history data and the tumor mass; and based on identifying the correlation between the patient history data and the tumor mass, storing, to the unified patient database, a fourth data object corresponding to the patient history data, the fourth data object connected to the first data object via the network of interconnected data objects.

In some aspects, the method further comprises receiving, from the diagnostic computer, tumor mass information corresponding to a tumor mass at a metastasis site of the primary cancer; analyzing the tumor mass information to identify a correlation between the diagnosis information and the tumor mass; and based on receiving the tumor mass information and identifying the first data object, storing, to the unified patient database, a fifth data object corresponding to the tumor mass information connected to the first data object via the network of interconnected data objects. In some aspects, the second data object includes one or more attributes selected from: a stage of the primary cancer, a biomarker, and a tumor size.

In some aspects, the method further comprises identifying, from the unified patient database, a data element and a data type associated with the patient; and transmitting, to an external system, the data element and the data type in structured form. In some aspects, the method, further comprises, upon generating each of the first data object and the second data object, generating a first timestamp stored in association with the first data object indicating the time of creation of the first data object and a second timestamp stored in association with the second data object indicating the time of creation of the second data object.

In some aspects, the method further comprises updating the unified patient database by: importing medical data from an external database; parsing the imported medical data to identify a particular data element associated with the patient and the primary cancer; and storing the particular data element to a sixth data object in association with the first data object.

In some aspects, the external database corresponds to at least one of: an EMR (electronic medical record) system, a PACS (picture archiving and communication system), a Digital Pathology (DP) system, an LIS (laboratory information system), and a RIS (radiology information system).

In some embodiments, a method of processing medical data to facilitate a clinical decision, comprising performing by a server computer: receiving, via a graphical user interface, identification data identifying a patient; receiving user input selecting a mode, of a set of selectable modes of the graphical user interface; based on the identification data and the user input, retrieving a set of medical data associated with the patient from a unified patient database, the set of medical data corresponding to the selected mode; and displaying, via the graphical user interface, a user-selectable set of objects in a timeline, the objects organized in rows, each row corresponding to a different category of a plurality of categories, the plurality of categories comprising pathology, diagnostics, and treatments.

In some aspects, retrieving the set of medical data comprises: querying a unified patient database to identify a patient record for the patient from the unified patient database, the patient record comprising a patient object; identifying each of a set of objects connected to the patient object; and retrieving a predetermined subset of the identified set of objects for display.

In some aspects, the set of medical data corresponds to one or more of: a treatment object in a unified patient database, the treatment object storing a treatment type, a date, and a response to the treatment; a diagnostic finding object in the unified patient database, the diagnostic finding object storing biomarker data, staging data, and/or tumor size data; and a history object in the unified patient database, the history object storing surgical histories, allergies, and/or family medical history.

In some aspects, the method further comprises detecting user interaction with an object of the set of objects; identifying and retrieving a corresponding report from the unified patient database; and displaying the report via the graphical user interface. In some aspects, the graphical user interface further comprises a ribbon displayed above the timeline, the ribbon displaying a subset of the objects flagged as significant.

In some aspects, the graphical user interface further comprises an element for navigating to a second interface view, the method further comprising: detecting user interaction with the element for navigating to the second interface view; and transitioning to the second interface view, the second interface view displaying oncologic summary data.

In some embodiments, a method for managing patient data comprises storing, to a unified patient database, a patient record, the unified patient database including data from a plurality of sources, the patient record including a plurality of data objects including a first primary cancer data object storing data elements corresponding to a first tumor mass of a patient and a second primary cancer data object storing data elements corresponding to a second tumor mass of the patient; rendering and causing display of a graphical user interface, the graphical user interface comprising a patient summary comprising information summarizing patient data in the patient record in the unified patient database; detecting user interaction with an element of the graphical user interface; responsive to detecting the user interaction, retrieving, from the unified patient database, the data elements from the first primary cancer data object and the second primary cancer data object of the patient record; and rendering: a first modal corresponding to a first primary cancer of a patient; and a second modal corresponding to a second primary cancer of the patient; and causing display of the first modal and the second modal side-by-side in the graphical user interface.

In some aspects, each of the modals displays a set of biomarkers with timestamps, staging information, and metastatic site information. In some aspects, the plurality of sources comprise two or more of: an EMR (electronic medical record) system, a PACS (picture archiving and communication system), a Digital Pathology (DP) system, an LIS (laboratory information system), a RIS (radiology information system), patient reported outcomes, a wearable device, or a social media website.

In some embodiments, a method of processing medical data to facilitate a clinical decision comprises receiving, via a portal, input medical data of a patient associated with a plurality of data categories, the plurality of data categories being associated with an oncology workflow operation; generating structured medical data of the patient based on the input medical data, the structured medical data being generated to support the oncology workflow operation to generate a diagnostic result comprising one of: the patient having no cancer, the patient having a primary cancer, the patient having multiple primary cancers, or the patient having a carcinoma of unknown primary sites; and displaying, via the portal, the structured medical data and a history of the diagnostic results of the patient with respect to a time in the portal, to enable a clinical decision to be made based on the history of the diagnosis results.

In some aspects, the portal comprises a data entry interface to receive the input medical data, and to map the input medical data into fields to generate the structured medical data; and wherein the data entry interface organizes the structured medical data into one or more pages, each of the one or more pages being associated with a particular primary tumor site. In some aspects, the method further comprises receiving, via the data entry interface, a first indication that a first subset of the medical data entered into a first page of the data entry interface associated with a first primary tumor site belongs to a second primary tumor site; and based on the first indication: creating a second page for the second primary tumor site; and populating the second page with the first subset of medical data.

In some aspects, the method further comprises receiving, via the data entry interface, a second indication that a second subset of the medical data entered into the first page is related to a metastasis of the second primary tumor site; and based on the second indication, populating the second page with the second subset of medical data. In some aspects, the method, further comprises importing a document file from a unified patient database; and extracting the input medical data from the document file based on at least one of a natural language processing (NLP) operation or a rule-based extraction operation on texts included in the document file.

In some aspects, the method further comprises displaying the document file in a document browser of the portal; and highlighting one or more portions of the document file from which the input medical data are extracted. In some aspects, the method, further comprises displaying one or more data fields next to the document browser; and displaying an indication that a subset of the one or more data fields are to be populated with the input medical data to be extracted from the highlighted one or more portions of the document file, to indicate a correspondence between the subset of the one or more data fields and the highlighted one or more portions of the document file.

In some aspects, the indication include emphasizing the subset of one or more data fields and encircling highlight markings over the highlighted one or more portions of the document file. In some aspects, the indication is displayed based on receiving an input from a user via the portal. In some aspects, the highlighted one or more portions are determined based on detecting an input from a user via the portal. In some aspects, the highlighted one or more portions are determined based on the at least one of the natural language processing (NLP) operation or the rule-based extraction operation.

In some aspects, the method further comprises determining one or more medical data categories of the extracted input medical data; determining a mapping between one or more fields in the structured medical data and the one or more medical data categories based on a structured data list (SDL); and populating the one or more fields with the extracted input medical data based on the mapping.

In some aspects, the mapping comprises mapping the input medical data to standardized values. In some aspects, the input medical data are received from one or more sources comprising at least one of: an EMR (electronic medical record) system, a PACS (picture archiving and communication system), a Digital Pathology (DP) system, an LIS (laboratory information system), a RIS (radiology information system), patient reported outcomes, a wearable device, or a social media website.

These and other embodiments of the invention are described in detail below. For example, other embodiments are directed to systems, devices, computer products, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

The detailed description is set forth with reference to the accompanying figures.

FIG. 1 illustrates a conventional clinical decision making process to be improved by examples of the present disclosure.

FIG. 2 illustrates a medical data processing system to facilitate a clinical decision, according to certain aspects of the present disclosure.

FIG. 3A, FIG. 3B, FIG. 3C, FIG. 3D, FIG. 3E, FIGS. 3F, 3G, and 3H illustrate examples of a data entry interfaces of the medical data processing system of FIG. 2, according to certain aspects of the present disclosure.

FIG. 4A, FIG. 4B, and FIG. 4C illustrate examples of a data abstraction interface of the medical data processing system of FIG. 2, according to certain aspects of the present disclosure.

FIG. 5A, FIG. 5B, FIG. 5C, and FIG. 5D illustrate examples of operations of the data abstraction interface of FIG. 4A-FIG. 4C.

FIGS. 6A, 6B, 6C, and 6D illustrate additional examples of data extraction interfaces and operations of the medical data processing system of FIG. 2, according to certain aspects of the present disclosure.

FIGS. 7A and 7B illustrate examples of data reconciliation interfaces and operations of the medical data processing system of FIG. 2, according to certain aspects of the present disclosure.

FIG. 8A, FIG. 8B, and FIG. 8C illustrate examples of a portal summary view that improves access to medical data of a patient, according to certain aspects of this disclosure.

FIG. 9A, FIG. 9B, FIG. 9C, FIG. 9D, and FIG. 9E illustrate examples of a portal patient journey view that improves access to medical data of a patient, according to certain aspects of this disclosure.

FIG. 10 illustrates an example of a portal reports view that improves access to medical data of a patient, according to certain aspects of this disclosure.

FIG. 11 illustrates an example of a portal performance metric view that improves access to medical data of a patient, according to certain aspects of this disclosure.

FIG. 12 illustrates an example of a data schema for patient data, according to certain aspects of this disclosure.

FIG. 13 illustrates another example of a data schema for patient data, according to certain aspects of this disclosure.

FIGS. 14A, 14B, 14C, and 14D illustrate an example overview workflow for patient data management, according to certain aspects of this disclosure.

FIG. 15 illustrates a method of managing patient data from disparate sources in a unified fashion, according to certain aspects of this disclosure.

FIG. 16 illustrates another method of managing patient data for improved access to the patient data, according to certain aspects of this disclosure.

FIG. 17 illustrates a method of displaying patient data via a graphical user interface for improved access to the patient data, according to certain aspects of this disclosure.

FIG. 18 illustrates a method of managing and displaying patient data, according to certain aspects of this disclosure.

FIGS. 19A and 19B illustrate an example of an oncology workflow enabled by the medical data processing system of FIG. 2, according to certain aspects of this disclosure.

FIG. 20A and FIG. 20B illustrate another example of an oncology workflow enabled by the medical data processing system of FIG. 2, according to certain aspects of this disclosure.

FIG. 21 illustrates a method of processing medical data to facilitate a clinical decision, according to certain aspects of this disclosure.

FIG. 22 illustrates an example computer system that may be utilized to implement techniques disclosed herein.

### DETAILED DESCRIPTION

Techniques are described for improving a clinician's access to patient data to perform a clinical decision, such as a clinical decision related to oncology. In some examples, a medical data processing system can collect medical data of a patient from multiple data sources, convert the medical data into structured data, and present the structured data in various forms, such as in a summary format, in a longitudinal temporal view report format, etc. The medical data processing system can also support an oncology workflow solution, which can support/perform a diagnosis operation on the collected medical data and present a result of the diagnosis to the clinician. The oncology workflow solution can enable a clinician, such as an oncologist or his/her delegates, to longitudinally manage cancer patients from suspicion of cancer through treatment and follow-up. A database and a graphical user interface for accessing the database are provided for updating and viewing patient data in oncology, e.g., representing a patient journey for diagnosis and/or treatment. The graphical user interface can, for example, be used by an oncologist to manage patient data and get a clear view of cancer progression and responsiveness to treatments over time.

In some examples, the medical data processing system includes a data collection module, a data abstraction module, an enrichment module, a data access module, and a data reconciliation module. The medical data collection module can receive or retrieve medical data of a patient. The patient data can originate from various data sources (at one or more healthcare institutions) including, for example, an EMR (electronic medical record) system, a PACS (picture archiving and communication system), a Digital Pathology (DP) system, a LIS (laboratory information system) including genomic data, RIS (radiology information system), patient reported outcomes, wearable and/or digital technologies, social media etc.

The database system can ingest data from multiple sources. For example, data can be ingested from one or more external databases, such as an Electronic Medical Record (EMR) repository, Picture Archiving and Communication System (PACS), etc., as noted above. Data can also be manually entered via fields in the user interface. The ingested data can include structured and unstructured data. The unstructured data may come from unstructured reports such as PDF files. In the case of unstructured reports, machine learning (e.g., Optical Character Recognition (OCR) and/or Natural Language Processing (NLP)) is used to identify and populate fields. Such as a database system that ingests data from multiple sources and stores the data within a new schema can be referred to as a unified patient database.

Within the unified patient database, the data can be stored in a graph structure, where data elements are linked to connect different cancers or other conditions in the patient with different treatments, observations, and so forth. The graph structure can also be used to link different cancers with one another (e.g. primary and metastasis).

Data can be ingested and enriched via the user interface. In particular, an interface is provided for data abstraction. In the data abstraction process, the information can be extracted from a report and used to populate fields of the interface, which a user can confirm or edit, to generate structured medical data. In the data enrichment process, enrichment operations are performed to improve the quality of the extracted medical data. Examples of enrichment operations include a normalizing various numerical values (e.g., weight, tumor size, etc.), replacing a non-standard terminology provided by a patient with a standardized terminology, filling in missing fields characterizing or supplementing data, which may involve displaying pull down menus including categories, data standardization formats, and the like. Automatically and/or via user input, fields are filled or updated. For example, the user can interact with interface elements to categorize a tumor as a primary cancer (also referred to as a primary tumor) or metastasis, or fill in other fields such as date, time, doctor's notes, etc.

Another interface view can be used for a reconciliation process. The reconciliation interface view may be triggered if data has been uploaded to the database but information is missing from the record such as an association with a primary cancer, a stage, or a surgery type. For example, in the reconciliation process, a tumor can be associated with one or more primary cancers, which may trigger the data record for the tumor being stored with an updated mapping in the unified patient database.

At any point in the data ingestion, abstraction, and reconciliation process, a patient journey can be viewed. The patient journey is a timeline showing various multi-modal elements of a patient's oncology journey and medical history in chronological fashion. This makes it easy to visualize patient cancer milestones and cancer progression (as it metastasizes, relapses, or recurs, for example). The patient journey includes a set of objects in a timeline. The objects can correspond to categories such as pathology, diagnostics, and treatments. Each category can have a row in the timeline, where objects in that category are displayed chronologically. Each object can be user-selectable. Upon detecting user interaction with an object, the system may retrieve and display supplementary information, reports, and the like via the graphical user interface.

Additionally, techniques can improve a clinician's access to patient data to perform a clinical decision, such as a clinical decision related to oncology. In some examples, the medical data processing system can collect medical data of a patient from multiple data sources, convert the medical data into structured data, and present the structured data in various forms, such as in a summary format, in a longitudinal temporal view report format, etc. The medical data processing system can support an oncology workflow, in which a clinician can perform various diagnoses at different stages of the workflow. The medical data processing system can facilitate entry of the diagnosis results at different stages of the workflow by a clinician, and perform post-processing of the data, both of which enable the clinician to longitudinally manage cancer patients from suspicion of cancer through treatment and follow-up. The medical data processing system can also support other medical applications, such as a quality of care evaluation tool to evaluate a quality of care administered to a patient, a medical research tool to determine a correlation between various information of the patient (e.g., demographic information) and tumor information (e.g., prognosis or expected survival) of the patient, etc. The techniques can also be applied to other types of diseases areas and not limited to oncology.

In some examples, the medical data collection module also provides a portal to allow inputting and displaying of structured medical data into the system. The structured medical data can include various information related to the diagnosis of a tumor, such as tumor site, staging, pathology information (e.g., biopsy results), diagnostic procedures, and biomarkers of both the primary tumor as well as additional tumor sites (e.g., due to metastasis from the primary tumor). The portal can display the structured data in the form of a patient summary. The portal can also organize the display of the structured data into pages, with each page being associated with a particular primary tumor site and including the fields of information of the associated primary tumor site and can be accessed by a tab. The data entry interface can allow a user to input medical data manually. Based on detecting the user's input of certain fields in the page of a first primary tumor (e.g., designation of an additional tumor site as a new primary tumor), medical data collection module can create an additional page for a second primary tumor, and populate the fields of the newly-created page for the second primary tumor based on the addition tumor site information input into the page of the first primary tumor. In some examples, the medical data collection module also allows a user to select an additional tumor mass found during a diagnostic procedure of the primary tumor and associate the mass with the second primary tumor to represent the case of metastasis. Based on detecting the association, medical data collection module can transfer all the diagnostic results of the additional tumor from the first primary tumor page to the newly-created page for the second primary tumor.

Moreover, the portal also allows a user to import a document file (e.g., a pathology report, a doctor note, etc.) from the aforementioned data sources. The medical data abstraction module can then perform a data abstraction operation, in which various medical data are extracted from the document file, and used to populate fields of the patient summary to generate structured medical data. In some examples, the medical data can be extracted based on performing, for example, a natural language processing (NLP) operation, a rule-based extraction operation, etc., on the texts included in the document file. In some examples, the medical data can also be extracted from metadata of the document file, such as date of the file, category of the document file (e.g., a pathology report versus a clinician's note), the clinician who authored/signed off the document file, and a procedure type associated with the content of the document file (e.g., biopsy, imaging, or other diagnosis steps). The extracted medical data can then be used to automatically populate various fields of the patient summary. The medical data abstraction module can also highlight parts of the document file from which the structured medical data are extracted, as well as the fields to be populated by the structured medical data, to allow a user to track/verify a result of the data abstraction operation. In some examples, the medical data abstraction module can also support manual extraction of structured medical data from the document file via the portal.

In addition, the enrichment module can perform various enrichment operations to improve the quality of the extracted medical data. One enrichment operation can include a normalization operation to normalize various numerical values (e.g., weight, tumor size, etc.) included in the extracted medical data to a standardized unit, to correct for a data error, or to replace a non-standard terminology provided by a patient with a standardized terminology based on various medical standards/protocols, such as International Classification of Diseases (ICD) and Systematized Nomenclature of Medicine (SNOMED). The enriched extracted medical data can then be stored in a unified patient database as part of the structured medical data (e.g., structured oncology data) for the patient. In addition, in a case where the portal receives medical data manually input by the user, the enrichment module can also control the portal to display pull down menus including alternatives of standardized data (e.g., SNOMED terminologies) which can be chosen by the user as input, to ensure that the user inputs standardized medical data into the medical data processing system.

The medical data abstraction module as well as the enrichment module can be continuously adapted to improve the extraction and normalization processes. For example, some of the original unstructured patient data from the data sources can be manually tagged to indicate mappings of certain data elements as ground truth. For example, a sequence of texts in doctor's notes can be tagged as ground truth indication of an adverse effect of a treatment. The tagged doctor's notes can be used to train, for example, an NLP of the data abstraction module, to enable the NLP to extract texts indicating adverse effects from other untagged doctor's notes. The NLP can also be trained with other training data sets including, for example, common data models, data dictionaries, hierarchical data (i.e. dependencies between/among text), to extract data elements based on a semantic and contextual understanding of the extracted data. For example, the natural language processor can be trained to select, from a set of standardized data candidates for a data element of the cancer registry, a candidate having a closest meaning as the extracted data. Moreover, some of the extracted data, such as numerical data, can also be updated or validated for consistency with one or more data normalization rules as part of the processing.

Further, the oncology workflow module can perform/support a diagnosis operation based on the structured medical data provided by the medical data collection module. In one example, the diagnosis operation can be performed to confirm the biopsy result is for the same primary tumor or is for a different tumor, and to track the size of the primary tumor for evaluating the tumor's response to particular treatment. In another example, the diagnosis operation can be performed to determine whether the patient has a single primary tumor site, multiple primary tumor sites, or unknown primary sites. The results of the diagnosis operation can then be recorded and/or displayed with respect to time in the portal as part of the medical journey of the patient, to enable an oncologist or his/her delegates, to longitudinally manage cancer patients from suspicion of cancer through treatment and follow-up. The diagnosis results can also be used to support other medical applications, such as a quality of care evaluation tool to evaluate a quality of care administered to a patient, a medical research tool to determine a correlation between various information of the patient (e.g., demographic information) and tumor information (e.g., prognosis or expected survival) of the patient, etc.

The disclosed techniques enable aggregation and extraction of medical data to generate a patient summary and display the data in a portal. By providing all the relevant medical data in a portal, and organizing the data according to tumor sites, the clinician's access of the medical data can be substantially improved, which in turn can facilitate the clinician's decision making and administering of care to the patient. In addition, as part of the oncology workflow, an automated diagnosis operation that mimics part of a clinician's diagnosis can be performed, which can reduce the clinician's work load. Moreover, the display of the diagnosis results, rather than the raw medical data, in the portal as part of the patient's journey can provide the clinician with better visualization of the medical states of the patient. This enables an oncologist or his/her delegates to longitudinally manage cancer patients from suspicion of cancer through treatment and follow-up. All these aspects can improve the quality of care provided to the patients.

### I. Clinical Decision Making

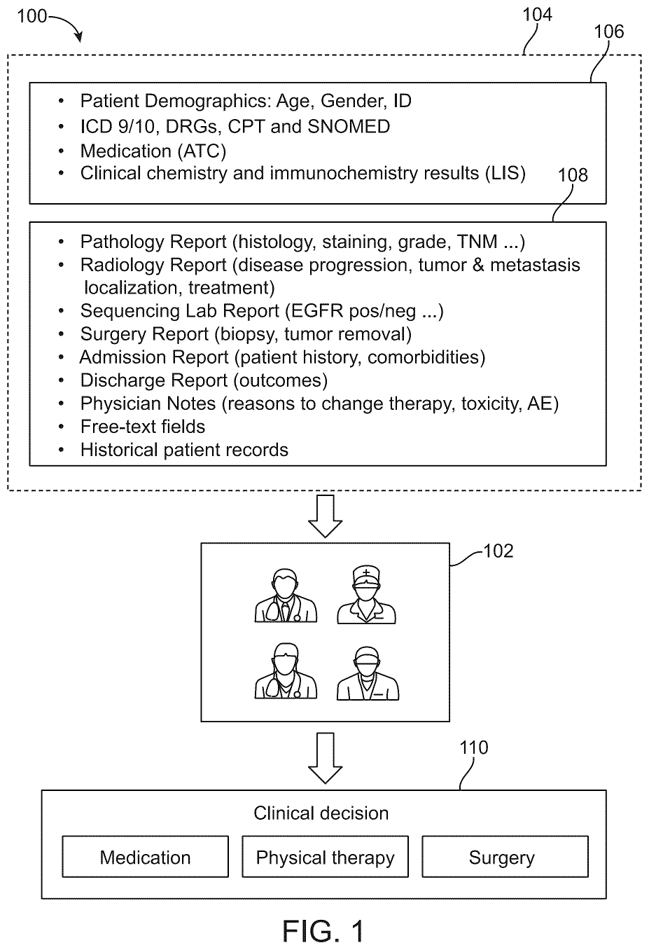

FIG. 1 is a chart 100 illustrating a conventional clinical decision making process. As shown in FIG. 1, clinicians 102 can obtain medical data 104 of a patient, which can include structured medical data 106 and unstructured medical data 108, to generate a clinical decision 110. Structured medical data 106 can include different categories of data including, for example, demographic information (age, gender, etc.) of the patient, diagnosis results described in terms of various standardized codes International Classification of Disease (ICD), Diagnosis-Related Group (DRG), Current Procedural Terminology (CPT) and SNOMED codes, medication history (e.g., Anatomical Therapeutic Chemical (ATC)), clinical chemistry and immunochemistry results, etc. In addition, unstructured medical data 108 can include different categories of data including various medical reports such as, for example, pathology reports, radiology reports, sequencing lab reports, surgery reports, admission reports, discharge reports, physician notes, etc. Clinical decision 110 may include, for example, medications, physical therapies (e.g., radiation), and surgeries to be administered to the patient. Medical data 104 is typically stored in different data sources, such as EMR (electronic medical record) system, PACS (picture archiving and communication system), Digital Pathology (DP) system, and LIS (laboratory information system).

Clinicians 102 may need to access each and every category of data listed in medical data 104 to make a decision. For example, clinicians 102 may need to access a pathology report and a surgery report to obtain information related to a tumor. Clinicians 102 may also need to access a radiology report to determine whether the tumor is localized or the cancel cells has spread and a sequencing lab report to obtain biomarker information. Clinicians 102 may also need to access physician notes to obtain information about, for example, a treatment history of the patient by another clinician. All these data are critical in deciding the treatment of the patient. For example, based on radiology report, the clinician can determine that the tumor is localized, and certain physical therapy (e.g., radiation therapy) can be administered to target at the localized tumor. Moreover, based on the presence of certain biomarkers, certain medication can be administered to target the site.

While clinicians 102 can have access to a large and diverse set of medical data to make a clinical decision, the procurement of the medical data from different data sources can be very laborious. The lack of structured and standardized medical data also makes the procurement difficult. For example, clinicians 102 need to read through and interpret numerous medical reports to obtain the information they are looking for. Clinicians 102 may also need to consider the habits of the physicians in writing the reports in order to interpret the reports properly. All these are not only laborious but also error-prone, which affect the clinician's capabilities in determining and administering high quality care to the patients.

### II. Medical Data Processing System

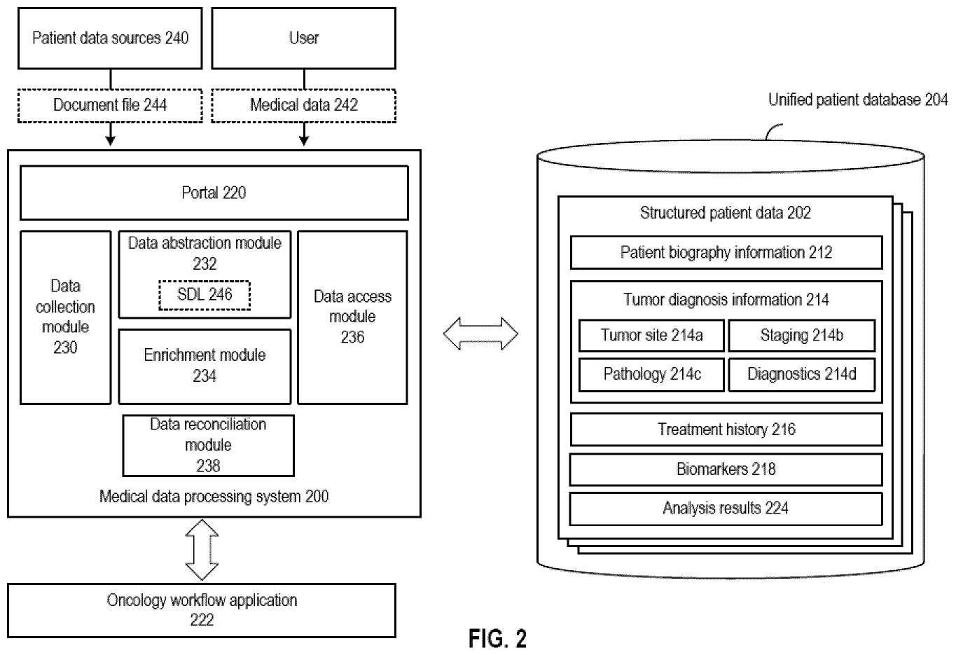

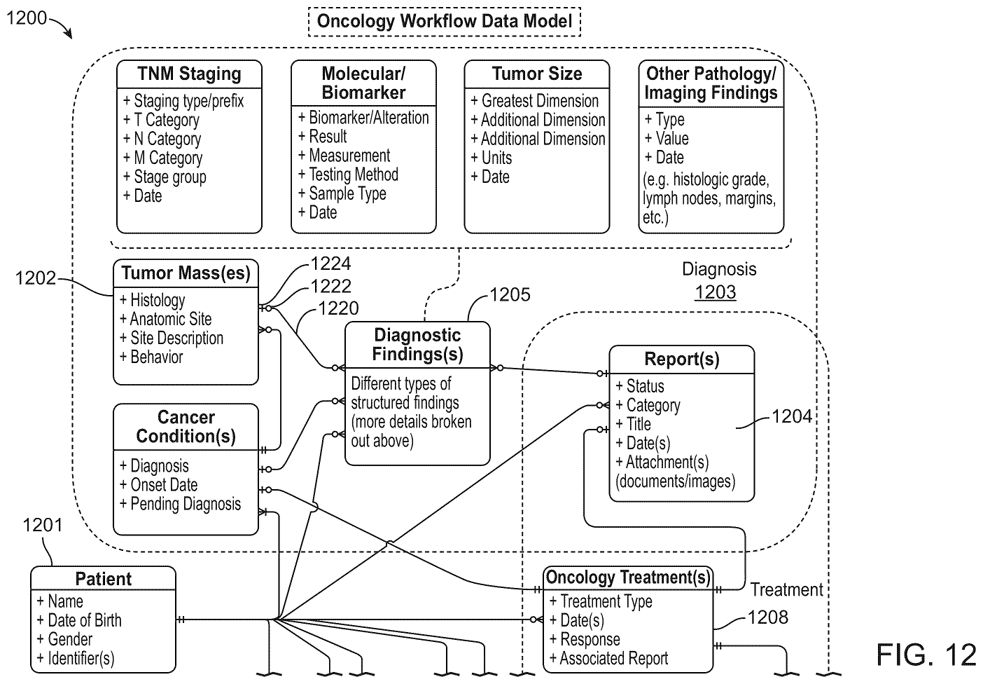

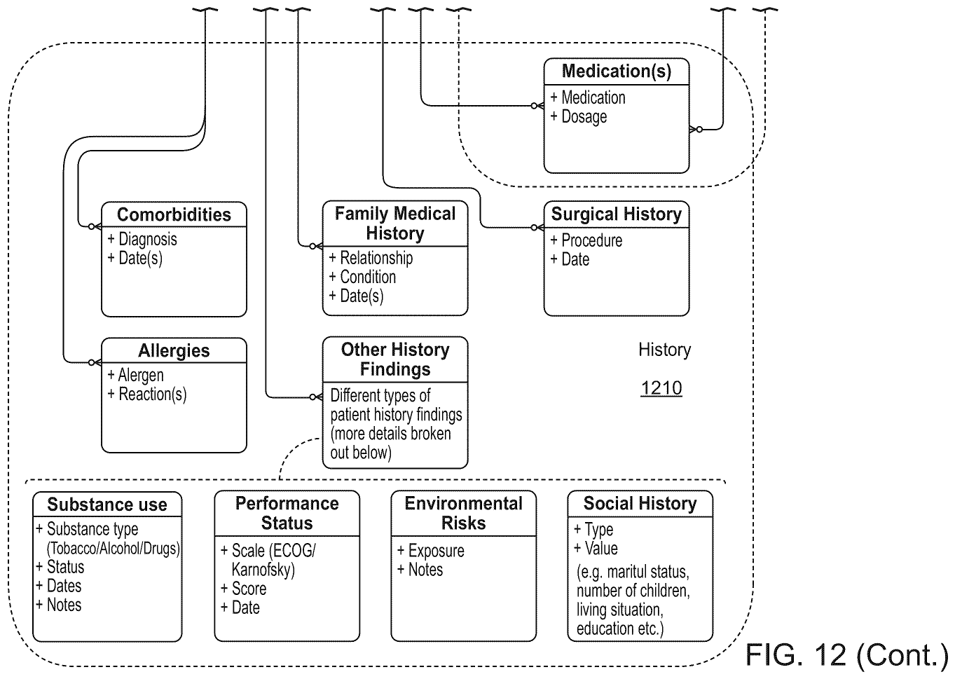

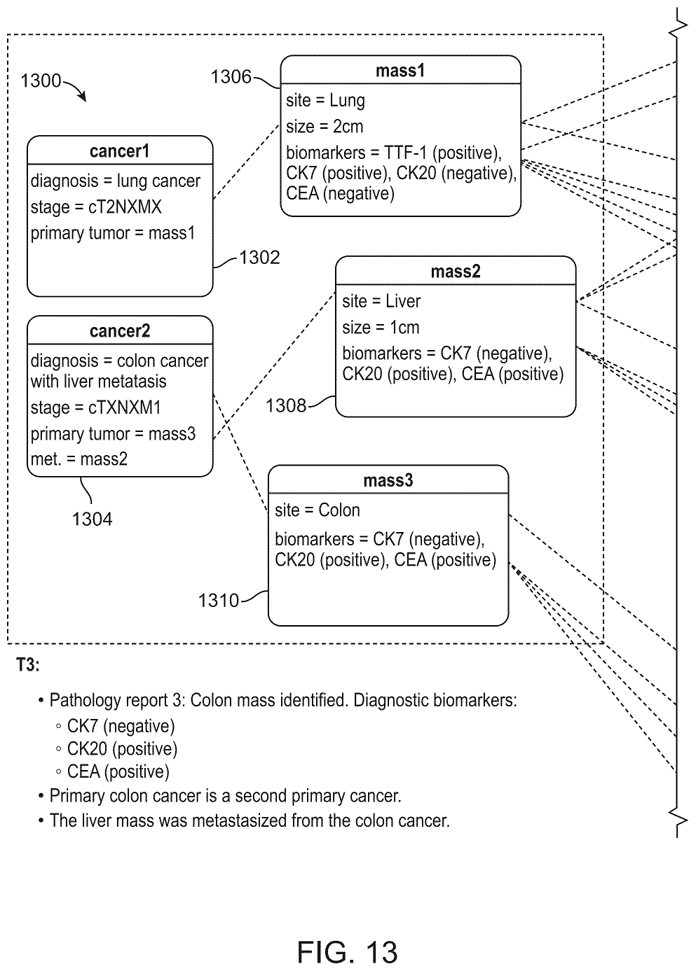

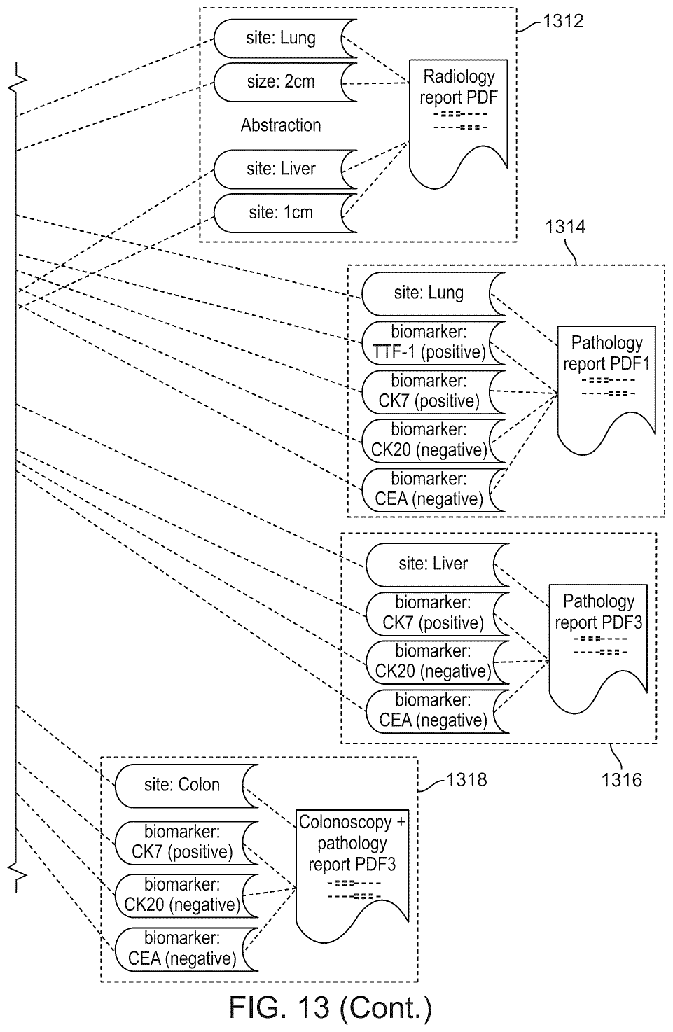

FIG. 2 illustrates an example of a medical data processing system 200 that can address at least some of the issues above. Medical data processing system 200 can collect medical data 242 of a patient and convert the medical data 242 into structured patient data 202. Medical data processing system 200 can also store structured patient data 202 to a unified patient database 204. The unified patient database 204 can store data retrieved from various sources in a unified fashion. The data may originate from one or more patient data sources 240. Patient data sources 240 may include one or more external databases or other sources, such as an Electronic Medical Record (EMR) repository, Picture Archiving and Communication System (PACS), a Digital Pathology (DP) system, a LIS (laboratory information system) including genomic data, RIS (radiology information system), patient reported outcomes, wearable and/or digital technologies, social media, and so forth. The data stored to the unified patient database 204 may include unstructured data such as PDFs or images of scanned documents, as well as information that was entered directly into the medical data processing system 200 via a portal 220. The unified patient database 204 can store multiple records, each corresponding to a particular patient. Each patient record can include network of interconnected data objects. Data schema for use in the unified patient database 204 are described in further detail below with respect to FIGS. 12 and 13.

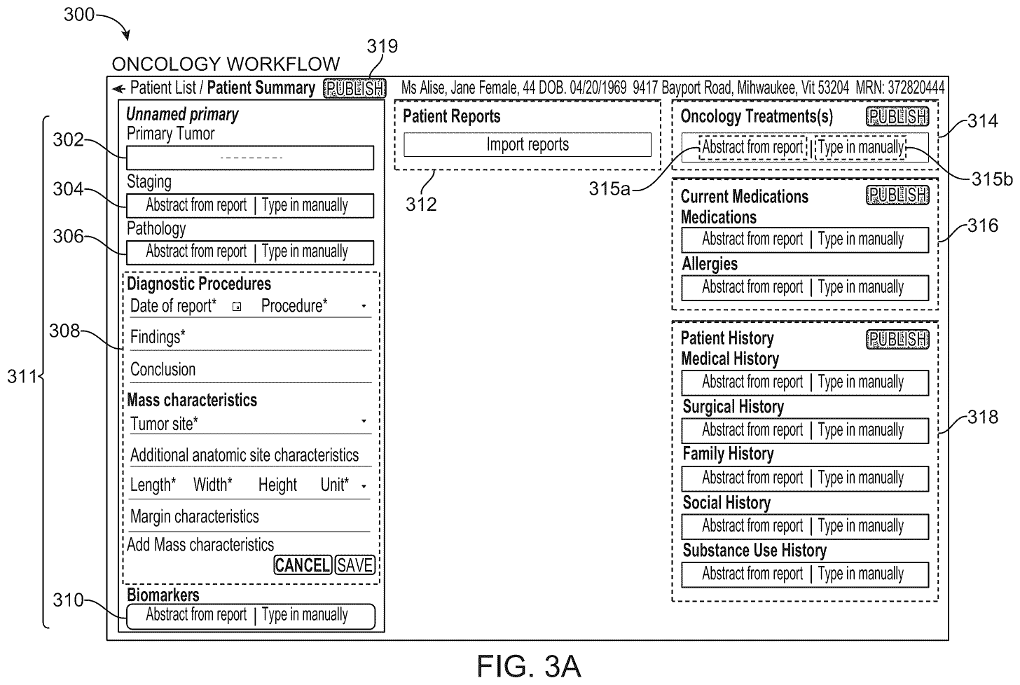

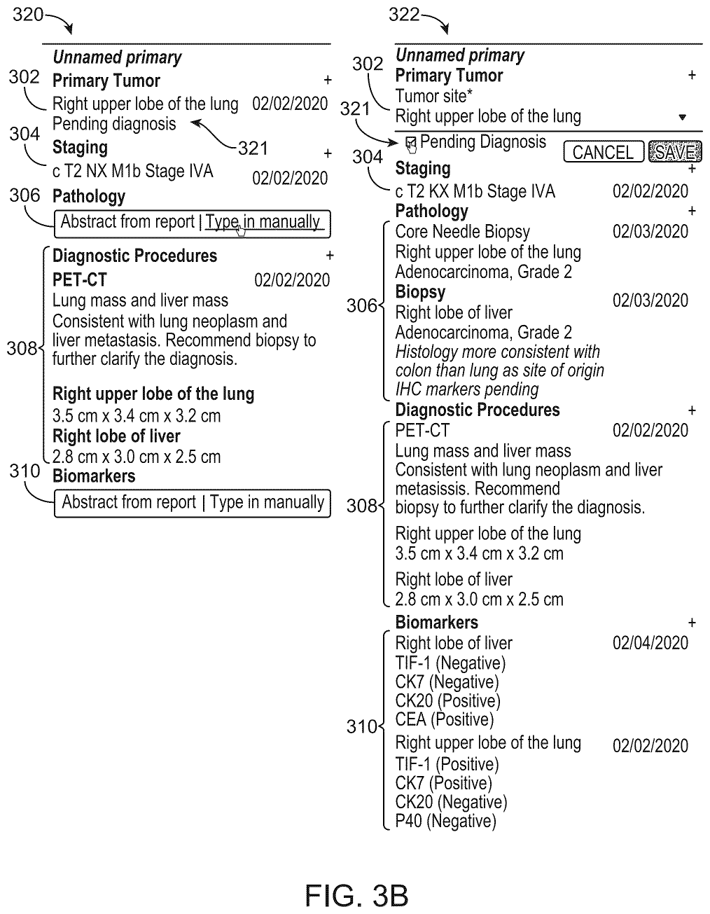

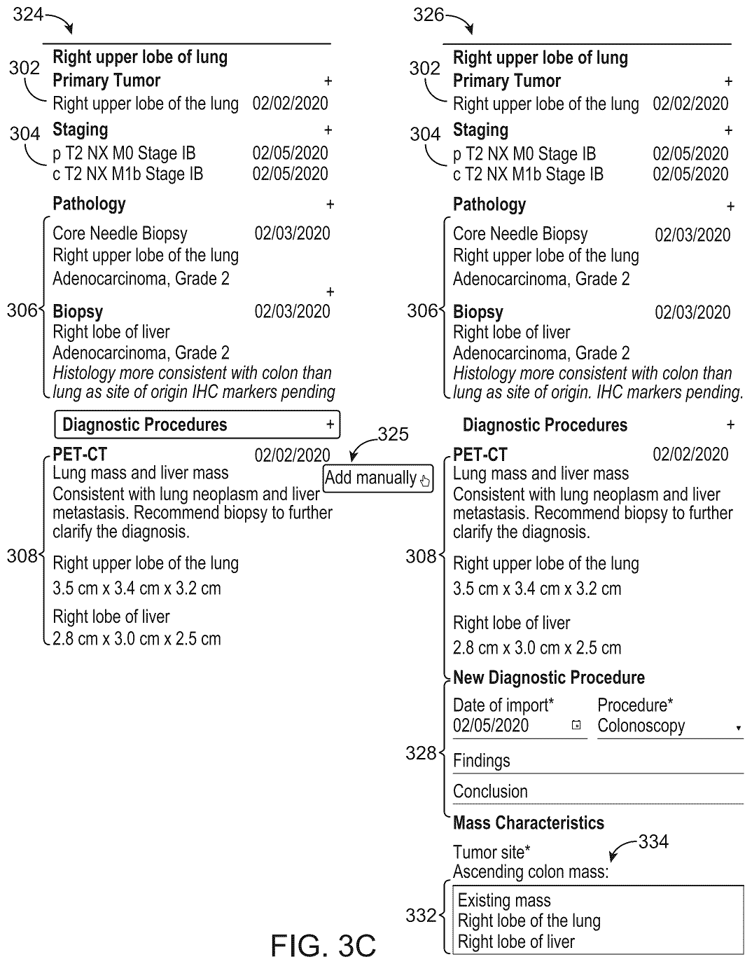

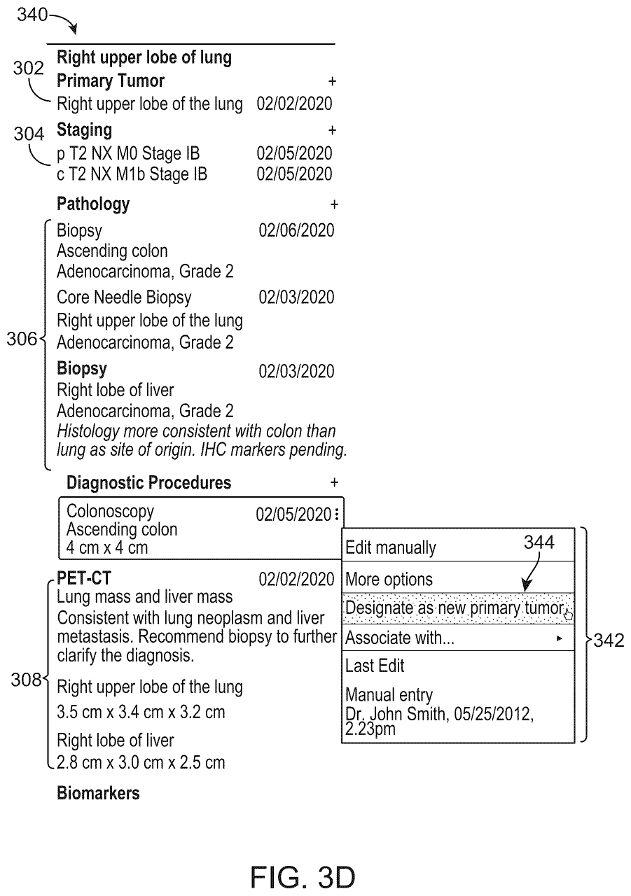

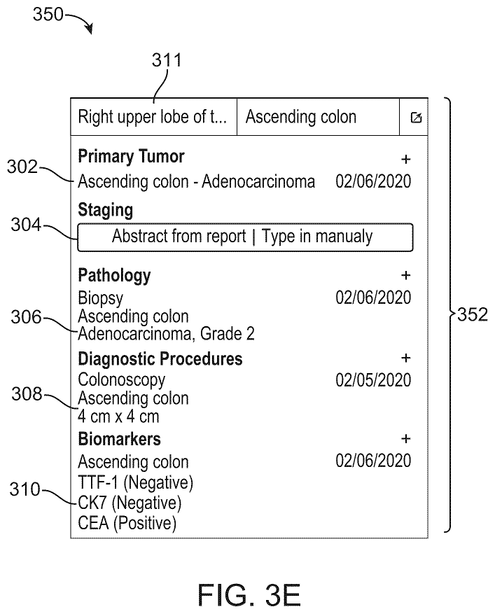

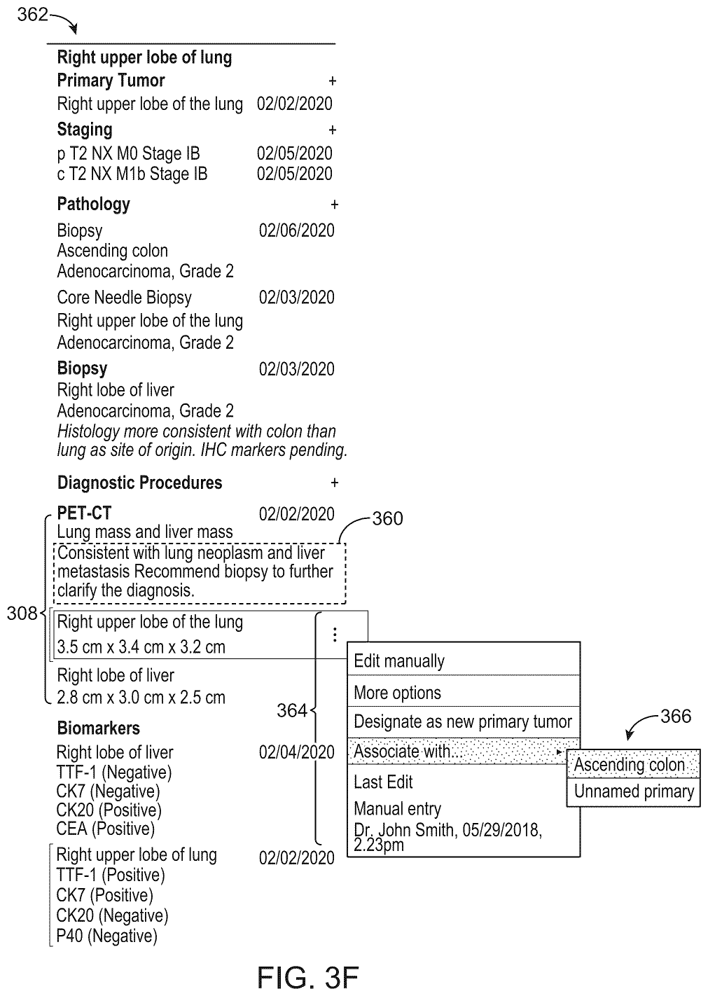

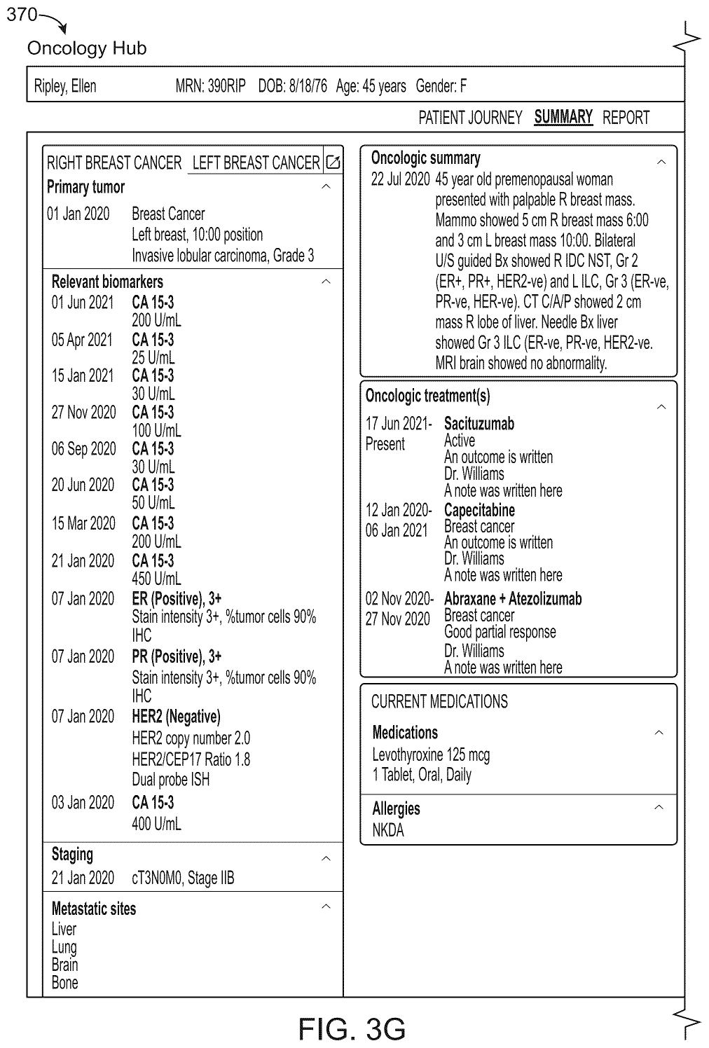

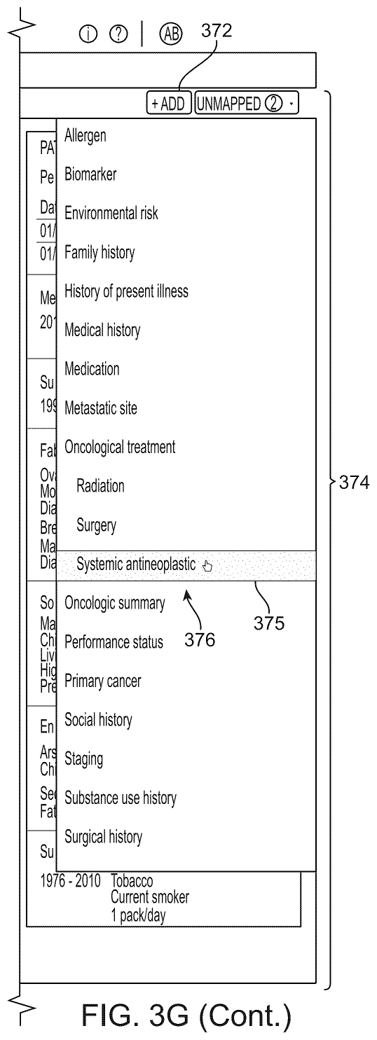

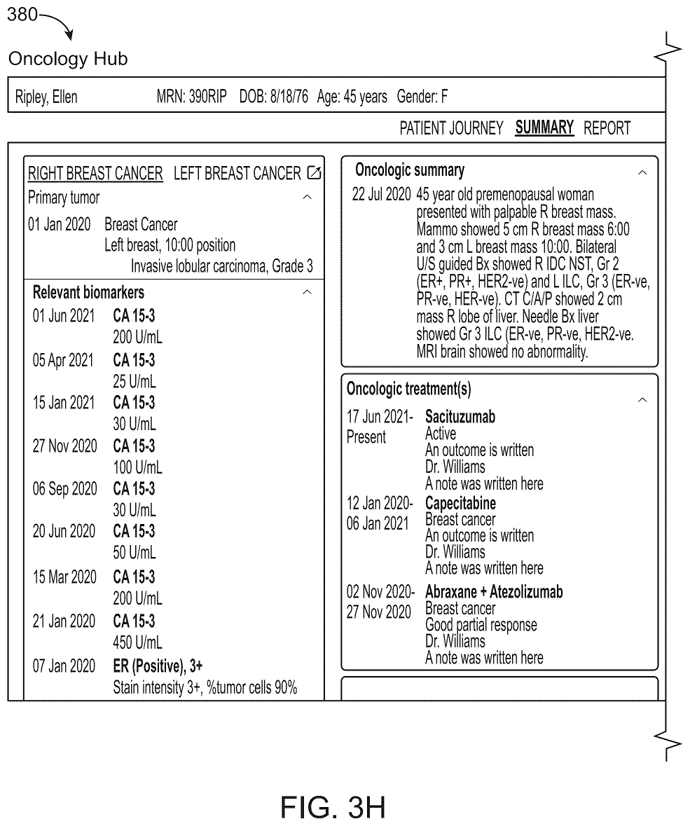

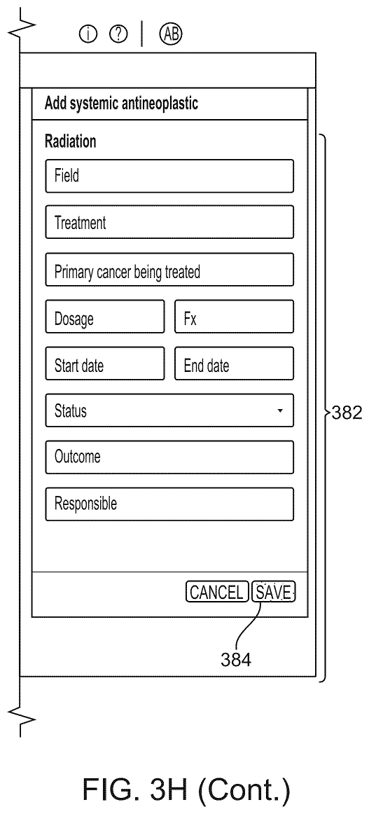

In a case where the medical data are directed to oncology, structured patient data 202 can include various data categories such as patient biography information 212, tumor diagnosis information 214, treatment history 216, and biomarkers 218. Tumor diagnosis information 214 can further include various data sub-categories or data types within a particular data category such as tumor site 214a, staging 214b, pathology information 214c (e.g., biopsy results), and diagnostic procedures 214d. Medical data processing system 200 further includes portal 220, which can present the structured data in various forms, such as in a summary format, in a longitudinal temporal view report format, etc., as illustrated in FIGS. 3A-11. In some implementations, portal 220 is displayed on a display component of a computing device separate from the medical data processing system 200. For example, a diagnostic computer (not pictured) displays the portal 220 and receives user input such as medical data 242.

In addition, medical data processing system 200 can support an oncology workflow application 222. Oncology workflow application 222 can determine data to be collected by medical data processing system 200 to support an oncology workflow. Moreover, as described below, oncology workflow application 222 can perform (or support) an analysis on the collected medical data and generate analysis results 224. The analysis can include determining a tumor state of the patient such as, for example, whether the patient has a single tumor or multiple tumors, whether the patient has metastasis, etc., based on structured patient data 202. The analysis result can be updated whenever new data (e.g., new diagnosis results, new biopsy results, etc.) is added for the patient. In some implementations, oncology workflow application 222 executes on a diagnostic computer.

The analysis result presented in portal 220 can enable a clinician, such as an oncologist or his/her delegates, to longitudinally manage cancer patients from suspicion of cancer through treatment and follow-up. The results of the diagnosis operation can then be recorded and/or displayed with respect to time in the portal as part of the medical journey of the patient. Portal 220 can enable an oncologist or his/her delegates to longitudinally manage cancer patients from suspicion of cancer through treatment and follow-up. The analysis results can also be used to support other medical applications, such as a quality of care evaluation tool to evaluate a quality of care administered to a patient, a medical research tool to determine a correlation between various information of the patient (e.g., demographic information) and tumor information (e.g., prognosis or expected survival) of the patient, etc. Medical data processing system 200 can store structured patient data 202, as well as analysis results 224 in unified patient database 204, from which the structured data and the analysis results can be accessed by other medical applications.

As shown, medical data processing system 200 includes a portal 220, a data collection module 230, a data abstraction module 232, an enrichment module 234, and a data access module 236. Data collection module 230 can receive medical data 242 from a user via a data entry interface of portal 220, in which the user can enter the data into various fields, and structured patients data 202 can be created via mapping between the fields and the entered data.

In addition, data collection module 230 can also receive medical data 242 directly from portal 220, which can provide a document abstraction interface that allows a user to import a document file 244 (e.g., a pathology report, a doctor note, etc.) from patient data sources 240. From document file 244, data abstraction module 232 can perform an abstraction operation, in which data abstraction module 232 extracts medical data from the document file and maps the extracted data to various data categories. The mapping can be based on a master structured data list (SDL) 246 that defines a list of data categories for a document type of document file 244 to support oncology workflow application 222. Patient data sources 240 (at one or more healthcare institutions) can include, for example, an EMR (electronic medical record) system, a PACS (picture archiving and communication system), a Digital Pathology (DP) system, a LIS (laboratory information system) including genomic data, RIS (radiology information system), patient reported outcomes, wearable and/or digital technologies, social media etc. After the abstraction operation, the user can edit and/or confirm the data extracted from the document.

In addition, enrichment module 234 can perform various enrichment operations to improve the quality of the extracted medical data, such as performing a normalization operation. The normalization operation can be performed to, for example, normalize various numerical values (e.g., weight, tumor size, etc.) included in the extracted medical data to a standardized unit, to correct for a data error, or to replace a non-standard terminology provided by a patient with a standardized terminology based on various medical standards/protocols, such as International Classification of Diseases (ICD) and Systematized Nomenclature of Medicine (SNOMED). As described below, enrichment module 234 can perform the normalization operation on the data received from data collection module 230 and/or data abstraction module 232. The enriched extracted medical data can then be stored to unified patient database 204 as part of the structured patient data 202 (e.g., structured oncology data) for the patient. Enrichment module 234 can also operate with portal 220 to provide interface elements such as a pull down menu including alternatives of standardized data which can be chosen by the user as input, to ensure that the user inputs standardized medical data into the medical data processing system.

Data access module 236 can provide a temporary storage of the data received from data collection module 230 and from data abstraction module 232 and update the data in the temporary storage based on the edits made to the data by the user through portal 220. Data access module 236 can release the data as structured patient data 202 to unified patient database 204 after receiving confirmation, through portal 220, from the user that the data is finalized and can be released back to unified patient database 204. Moreover, data access module 236 can provide various applications, such as oncology workflow application 222, with access to the data in the temporary storage. This can provide the user with information to track and manage the data entry and data abstraction operations, at data collection module 230 and data abstraction module 232, that supports the workflow application.

Data reconciliation module 238 can identify data elements in the unified patient database 204 that are missing information needed to properly store and display patient data. For example, if a data record for a particular cancer mass is not associated with a primary cancer site, this cancer mass can be flagged for reconciliation. The data reconciliation module 238 can provide UI elements that prompt a user to enter the necessary information (e.g., to associate a cancer mass with a primary cancer, e.g., as a new primary cancer or as a metastasis of another primary cancer). The data reconciliation module 238 can retrieve user input and modify the data record for the cancer mass to associate the cancer mass with the primary cancer identified via the user input to the UI.

### III. Example Interfaces

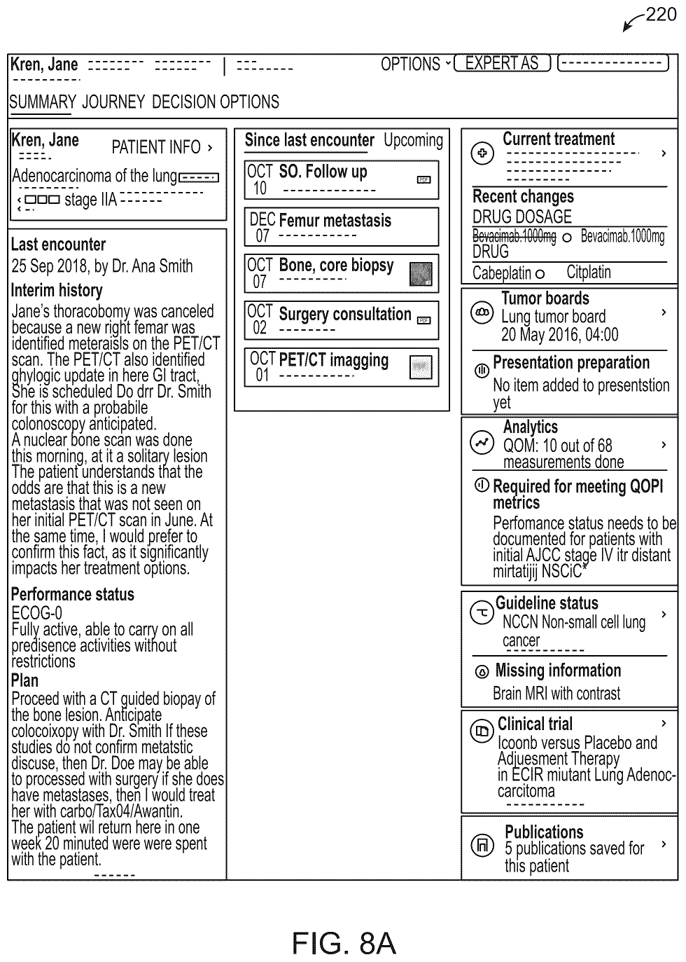

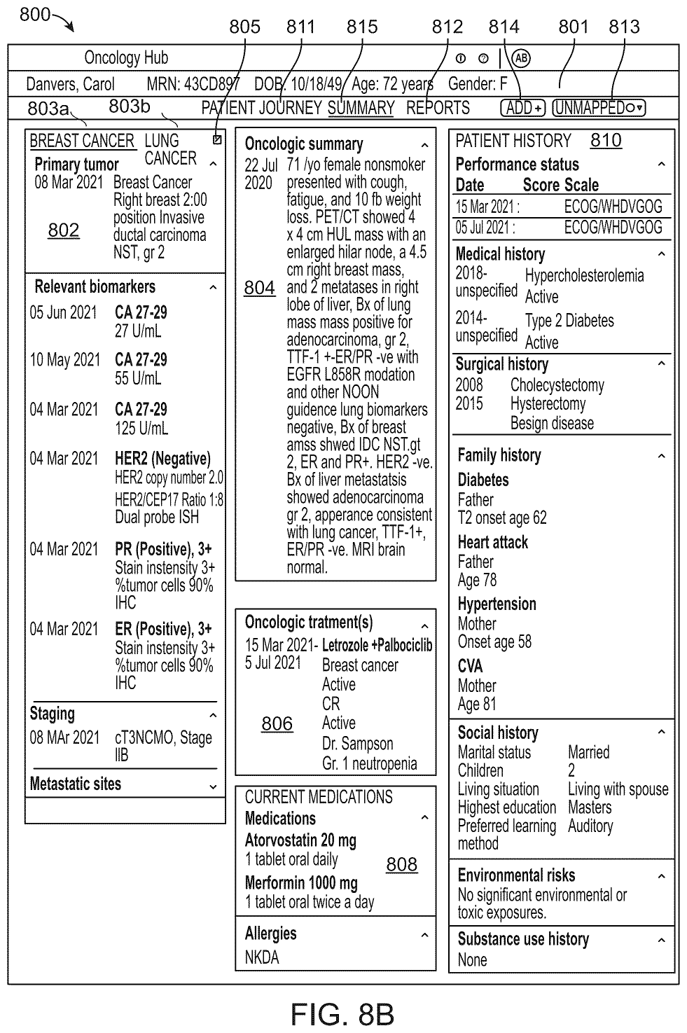

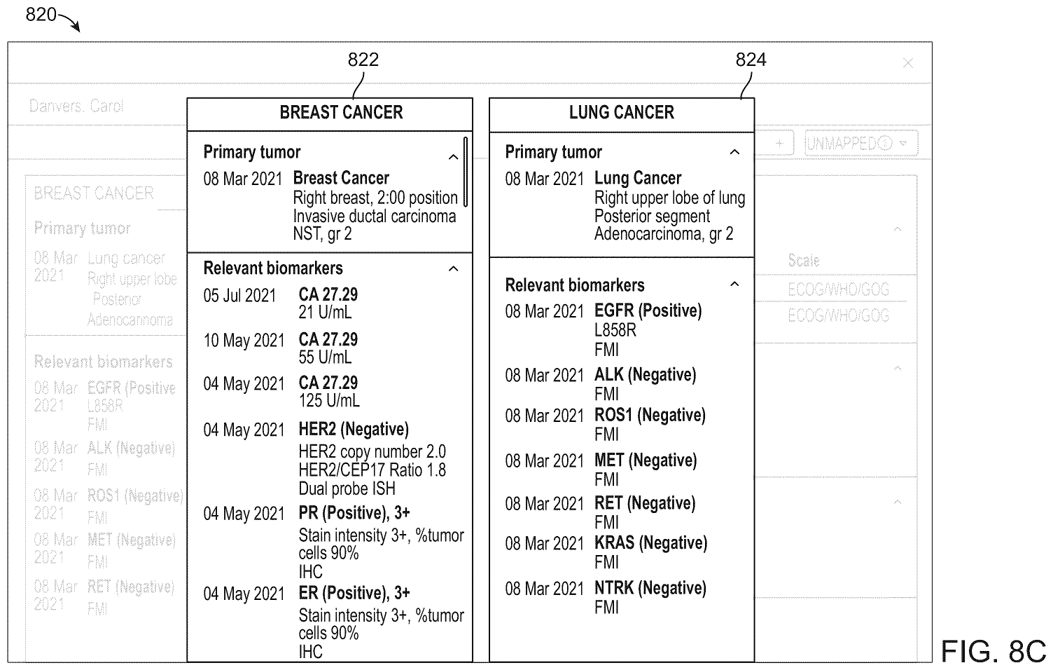

FIGS. 3A-11 illustrate various interfaces that can be used to display patient data and facilitate ingestion and organization of patient data for clinical decision making. The data entry interfaces of FIGS. 3A-7B can be used to import and organize data to be stored in the unified patient database. The view interfaces of FIGS. 8A-11 can be used to retrieve and display data from the unified patient database for use in clinical decision making.

A. Data Entry Interfaces

FIG. 3A, FIG. 3B, FIG. 3C, FIG. 3D, FIG. 3E, FIG. 3F, FIG. 3G, and FIG. 3H illustrate examples of portal 220. The examples provide an interface for managing medical data for an example patient.

1. Summary Page

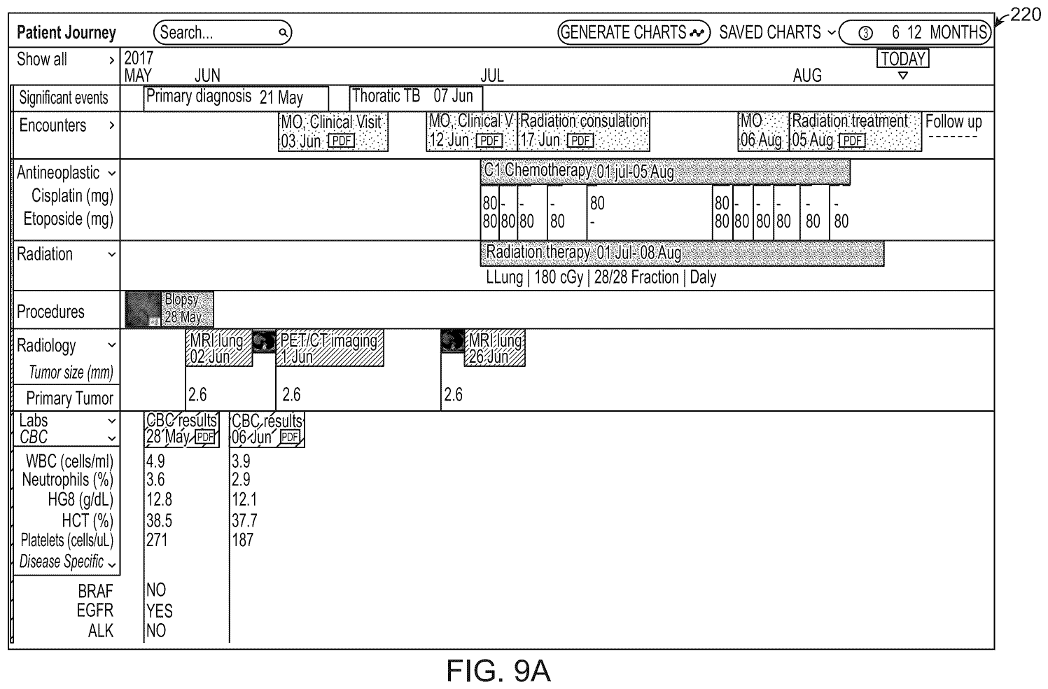

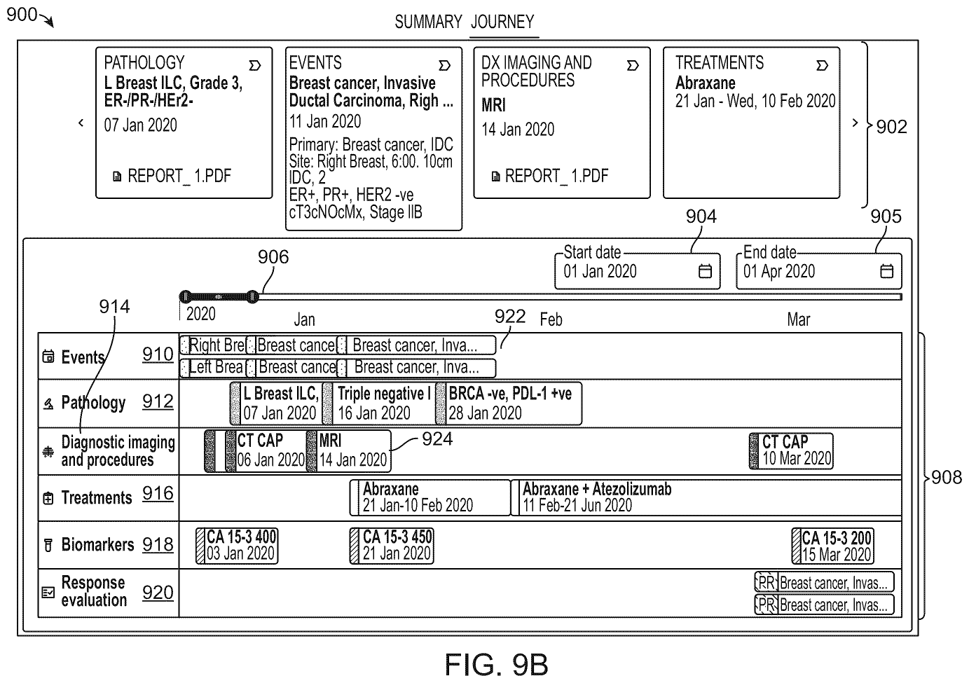

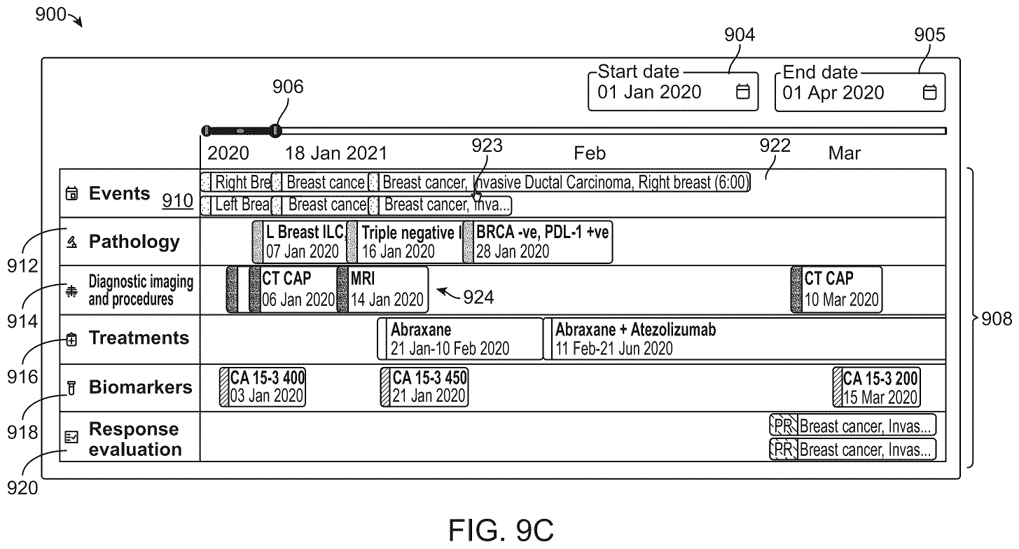

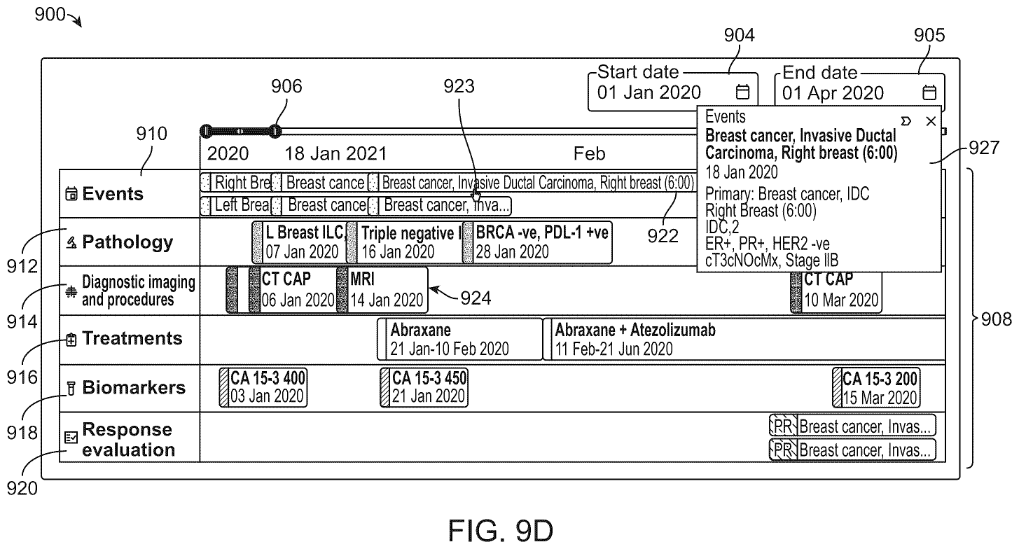

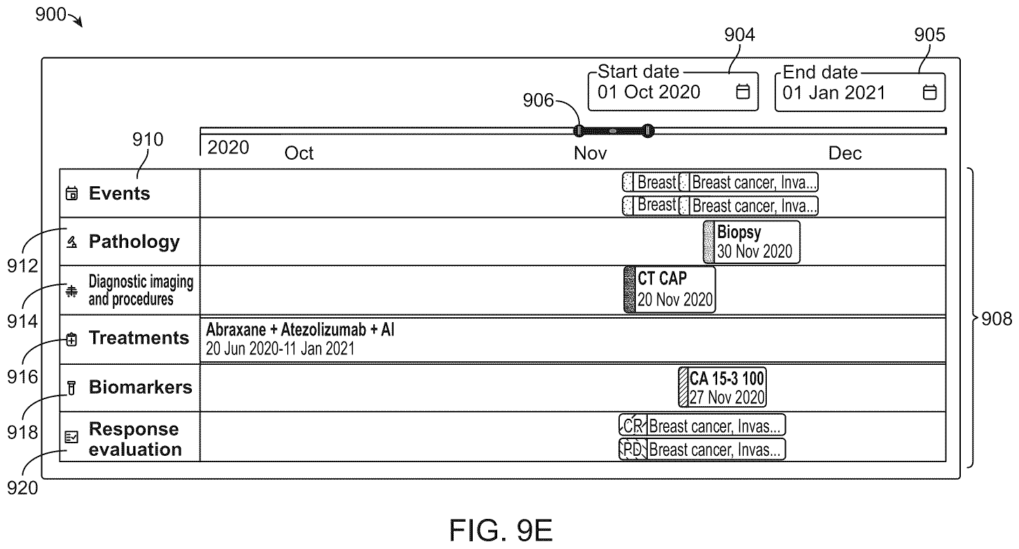

As shown in FIG. 3A, portal 220 can provide a data entry interface 300 to enter data to support oncology workflow application 222. Data entry interface 300 can guide a user to enter data manually and/or approve or edit automatically extracted data. Data received via data entry interface 300 of portal 220 can be stored based on fields 308 of data entry interface 300 in an appropriate fashion to unified patient database 204 using data schema such as those described below with respect to FIGS. 12 and 13. Data from unified patient database 204 can then be retrieved for displaying further interface views such as a patient journey view showing a longitudinal temporal view report of patient data over time, as shown in FIGS. 9A-9E.

Data entry interface 300 includes various fields for various information related to the diagnosis of a tumor, such as a field 302 for tumor site, a field 304 for staging, a field 306 for pathology information (e.g., biopsy results), fields 308 for diagnostic procedures, and field 310 for biomarkers. Fields 302-310 can form a patient summary page 311 for a particular tumor site. In addition to patient summary page 311, data entry interface 300 can include fields for other information, such as patient reports 312, oncology treatment information 314 about a set of oncology treatments the patient has received, current medications information 316 about the current medications received by the patient, and patient history information 318 about the various histories (e.g., medical history, surgical history, family history, social history, and substance use history) of the patient. Data entry interface 300 provides an interface to aggregate different modalities of patient data and then convert the data into structured patient data 202. The fields and various options provided in data entry interface 300 can be defined based on oncology workflow application 222.

Each of patient summary page 311, patients report 312, oncology treatment information 314, current medications information 316, and patient history information 318 further includes a publish button. For example, patient summary page 311 includes a publish button 319. As described above, as data entry interface 300 receives data entered into the various fields, data access module 236 can store the data in the temporary storage and withhold the data from unified patient database 204. The activation of publish button 319 can prompt data access module 236 to send the data as structured patients data 202 to unified patient database 204.

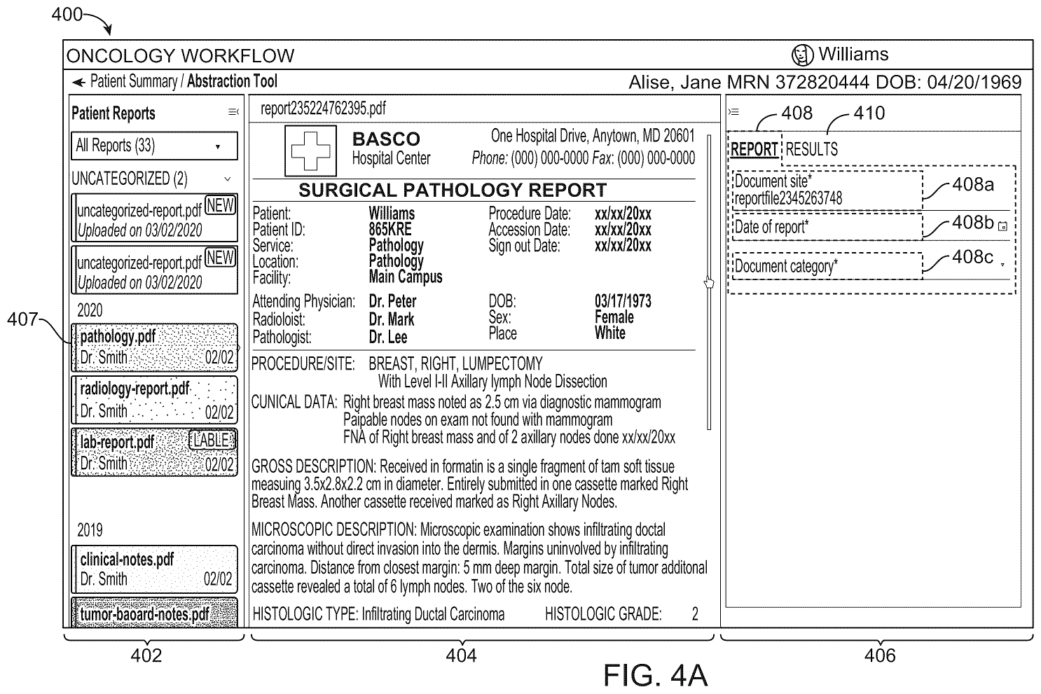

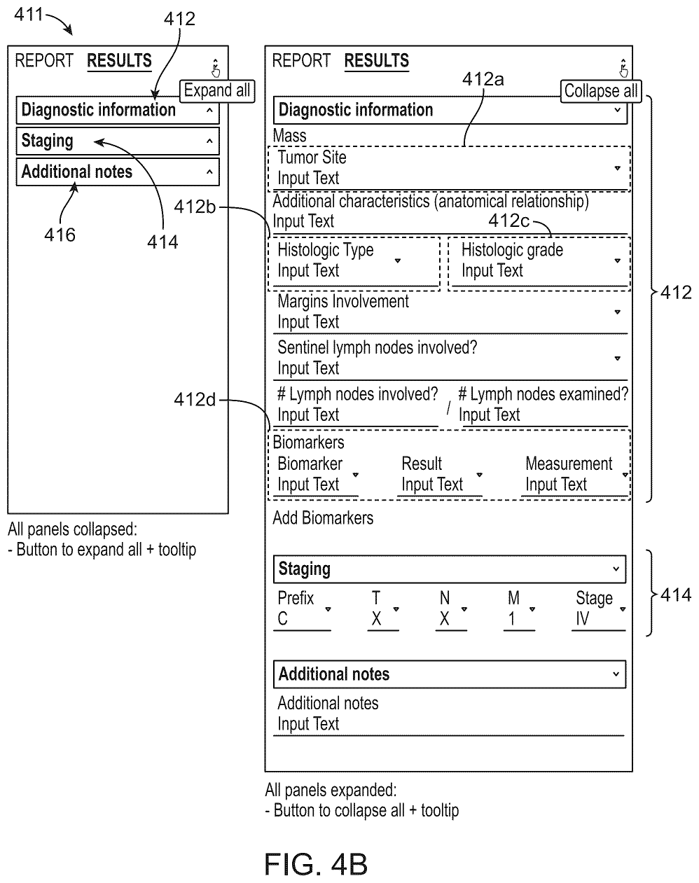

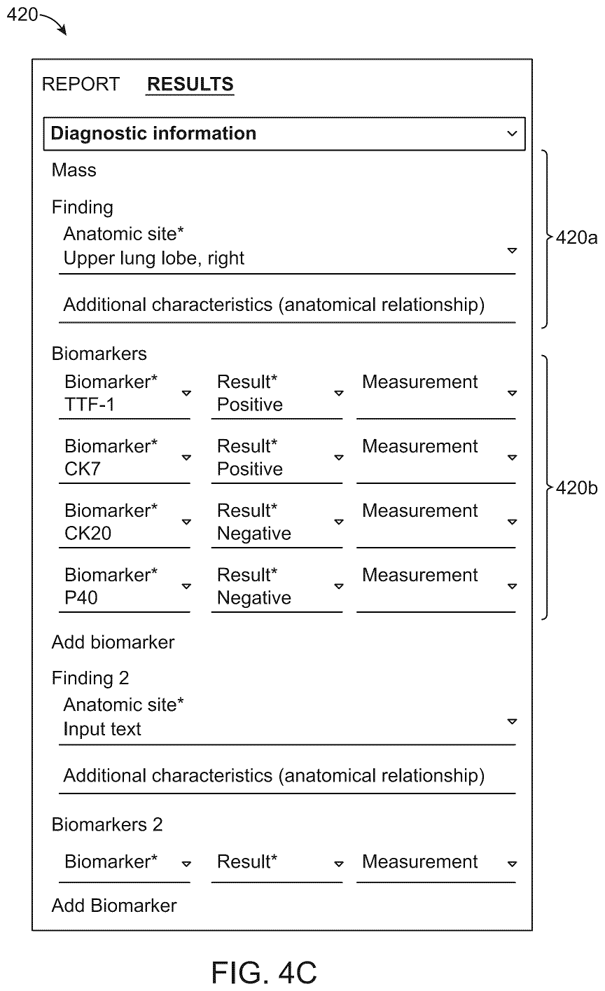

Data entry interface 300 can provide various ways to enter data for most of the fields, including manual entry of data, and abstraction from a document file. For example, in the field for oncology treatment information 314, a link 315a and a link 315b can be provided. Activation of link 315a can lead to display of a data abstraction portal (e.g., as described below with respect to FIGS. 4A-6D) to extract the data for oncology treatment information from a document field, whereas activation of link 315b can lead to display of a text box and/or a pull-down menu to allow the user to manually enter the data for oncology treatment information, as now described with respect to FIGS. 3B-3F.

2. Operations of Summary Page

FIG. 3B-FIG. 3F illustrate examples of operations of patient summary page 311 in receiving data manually input by a user. Referring to operation 320 of FIG. 3B, primary tumor field 302 can receive input text “right upper lobe of the lung” (e.g., a location), but the diagnosis is not yet confirmed and is still pending, and “pending diagnosis” flag 321 is asserted. The title of patient summary page 311 remains “Unnamed Primary.” Moreover, diagnostic procedures field 308 can receive the input text indicating that Positron Emission Tomography-Computed Tomography (PET-CT) is performed as part of the diagnostic procedures, and masses consistent with lung neoplasm and liver metastasis are found. The input text further indicates the sizes of masses found in the lung and in the liver. In operation 322, in primary tumor field 302, “pending diagnosis” flag 321 is de-asserted to confirm that the mass in the right upper lobe of the lung is a primary tumor. In addition, additional information is input to pathology field 306. Such designations may be imported by medical data processing system 200 and stored to unified patient database 204 according to the structured fields established via the interface.

Referring to operation 324 of FIG. 3C, after detecting that the “pending diagnosis” flag is de-asserted, data entry interface 300 can change the title of patient summary page 311 from “Unnamed Primary” to “Right upper lobe of the lung” to reflect that the information in fields 302-310 belong to a tumor in the right upper lobe of the lung. Moreover, referring to operation 326 of FIG. 3C, upon detecting that an add icon 325 is activated, data entry interface 300 can display an additional sets of fields for the user to enter information about a new diagnostic procedure. The information may include, for example, the date of the new diagnostic procedure, the name of the procedure, and the findings. Moreover, a pull-down menu 332 is provided to select the site of the tumor mass found in the new diagnostic procedure for fields 334. The candidates listed in pull-down menu 332 can be provided as standardized terminologies by enrichment module 234 so that only standardized terminologies are input into fields 334. As shown in FIG. 3C, in operation 326, an additional tumor mass (ascending colon mass) is added as a result of the new diagnostic procedure.

FIG. 3D, FIG. 3E, and FIG. 3F illustrate examples of operations to create a new page for a second primary tumor after page 311 (for the primary tumor at right upper lobe of the lung) is populated with data. Referring to FIG. 3D, in operation 340, data entry interface 300 can provide a pull-down menu 342 upon detecting that the additional tumor mass listed in the new diagnostic procedure is selected. Pull-down menu 342 includes an option 344 that allows a user to designate the newly added tumor mass (ascending colon mass) as a new primary tumor. Referring to FIG. 3E, in operation 350, upon detecting the selection to designate the newly added tumor site in the colon as a new primary tumor, data entry interface 300 can create a new page 352 for the primary tumor at the ascending colon, in addition to page 311 for the primary tumor at the right upper lobe of lung. Enrichment module 234 can also add in the standardized terminology “Adenocarcinoma” in the primary tumor site information for page 352 as a supplement to ascending colon. In addition, fields 302-310 of page 352 are populated with information from page 311, such as new diagnostic procedures added back in operation 326 of FIG. 3C. As a result of operation 340, data collection module 230 can create, as part of structured patient data 202 for a patient, a first data structure for a primary tumor site in the right upper lobe of lung and a second data structure for a primary tumor site in the ascending colon, with each data structure including a set of tumor diagnosis information, treatment history, and biomarkers.

After page 352 for the second primary tumor site (ascending colon) is created, certain diagnostic results for page 311 (for the primary tumor at right upper lobe of the lung) can be linked with the second primary tumor site. For example, referring to FIG. 3F, the diagnostic results for page 311 include information 360 of an additional tumor mass in the right upper lobe of the lung. In operation 362, data entry interface 300 can detect the selection of information 360 and output a menu 364, which includes an option 366 of associating with the additional tumor mass with the second primary tumor site ascending colon. Upon detecting a selection of option 366, data collection module 230 can move information 360 into page 352 for the second primary tumor site, to indicate that the additional tumor mass at right upper lobe of the lung is the result of metastasis at the second primary tumor site of ascending colon.

3. Adding Various Categories of Medical Data

FIG. 3G illustrates a patient summary view 370 of the portal 220. The patient summary view 370 is a view of a graphical user interface for viewing and modifying data for a patient. The patient summary view 370 includes an add button 372. Responsive to detecting user interaction with the add button 372, an add data modal 374 is displayed. Add data modal 374 can be a web page element that displays in front of other page content. Add data modal 374 may deactivate page content outside of add data modal 374 while displayed. Add data modal 374 includes a list of data types and data categories for which data can be entered and stored. The data types and data categories shown in FIG. 3G include allergen, biomarker, environmental risk, family history, history of present illness, medical history, medication, metastatic site, oncological treatment, radiation, surgery, systemic antineoplastic 375, oncologic summary, performance status, primary cancer, social history, staging, substance use history, and surgical history. A data category may include data types within that data category. For example, radiation, surgery, and systemic antineoplastic 374 are data types within the data category of oncological treatment in this example. The data types and data categories shown in add data modal 374 may correspond to data objects stored in a map structure in the unified patent database, where the data types and data categories label and organize corresponding data elements. For example, the data objects can include a patient root data object 1201, mapped to associated data objects including a tumor mass data object 1202, a diagnostic findings data object 1205, treatment data objects 1208, and history data objects 1210, as depicted in FIG. 12. This data schema facilitates display of the patient summary view, and information entered via the patient summary view can be used to modify the data in the unified patient database, as further described below in section IV.

Each of these data types and data categories can correspond to a different set of configured data fields. Responsive to user interaction with one of the displayed data types or data categories, the portal 220 can transition to a data entry view 380, including the data fields corresponding to the selected data type, as depicted in FIG. 3H. As shown in FIG. 3G, a cursor 376 indicates user interaction with the displayed data type systemic antineoplastic 375. On hover, systemic antineoplastic 375 is highlighted. Clicking systemic antineoplastic 375 causes the interface to transition to the data entry view 380 including the data fields corresponding to systemic antineoplastic 375.

FIG. 3H illustrates a data entry view 380 of the portal 220 according to some embodiments. The data entry view 380 can be used to receive medical data for a patient via the portal 220. The data is stored to the unified patient database in a patient record, which may be organized in a data graph mapping the data elements (e.g., as entered into the interface) to one another based on the configured data types as shown in FIG. 12. A menu 382 includes a set of fields that can accept user input to manually provide information corresponding to respective fields. These fields can include both drop-down menus, from which a type of treatment, primary cancer, status, or outcome can be selected, and fields configured to accept typed user input such as a number of cycles, start date, end date, responsible party, and additional notes. Responsive to detecting user interaction with a save button 384, the system saves the data input to the fields. For example, the data element input into each field can be saved to the unified patient database 204, organized based on a data type corresponding to that field.

B. Interfaces for Managing Data Ingestion from Unstructured Reports

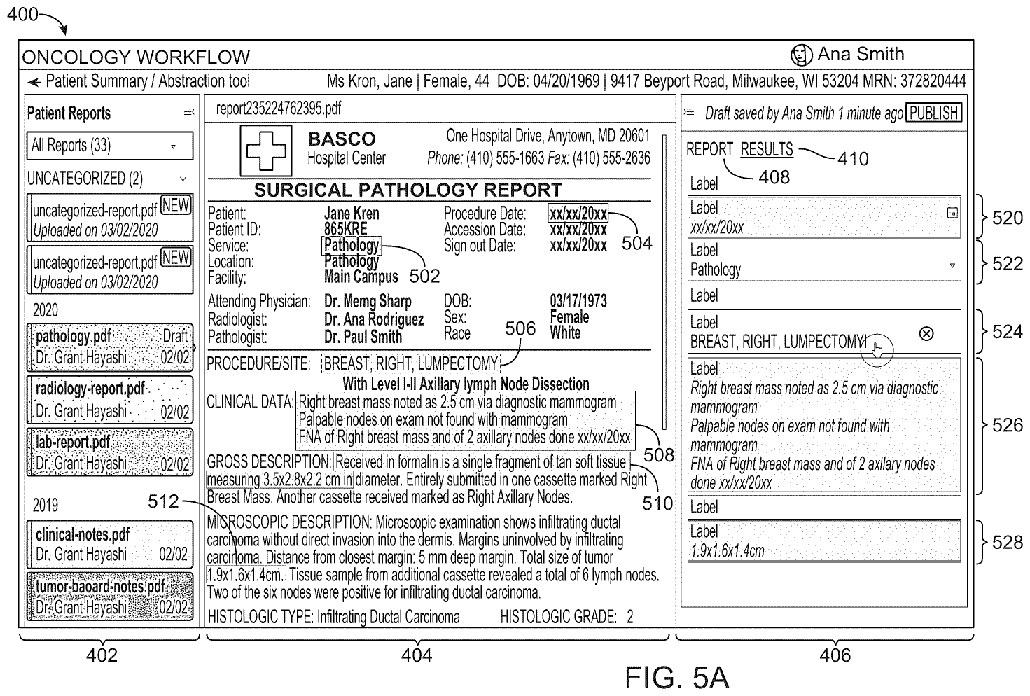

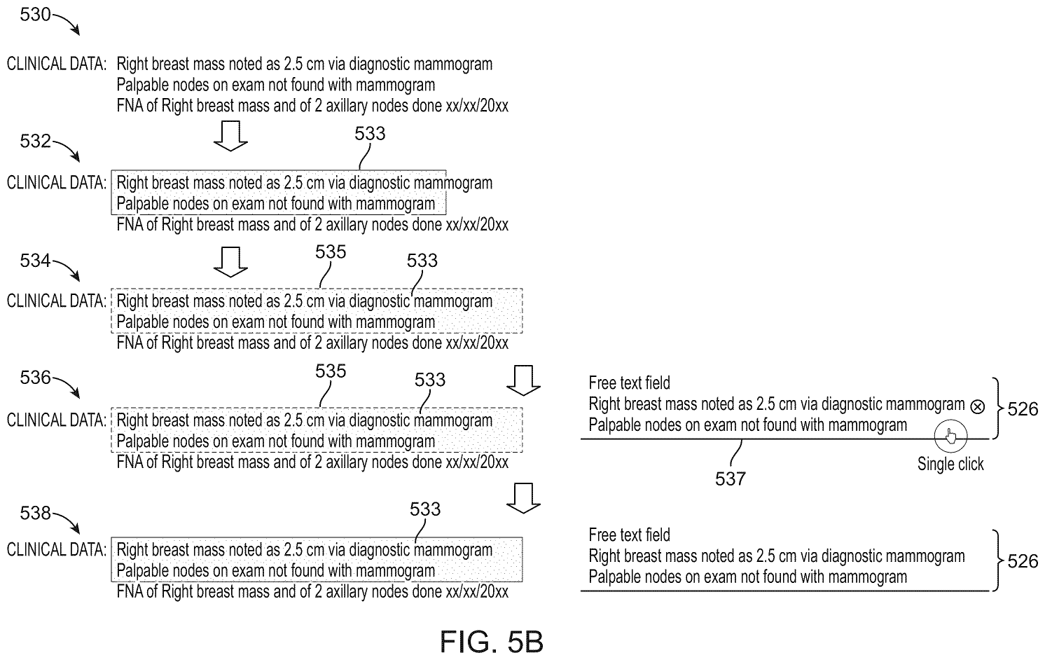

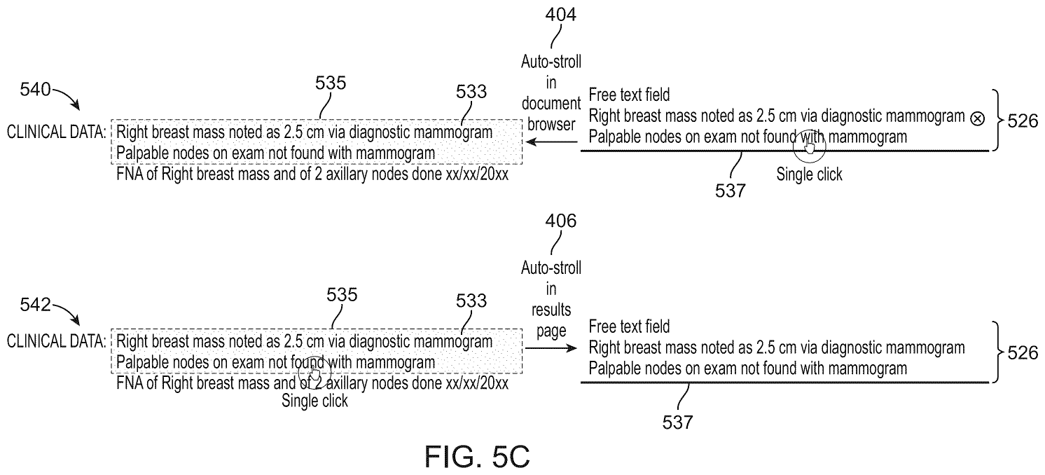

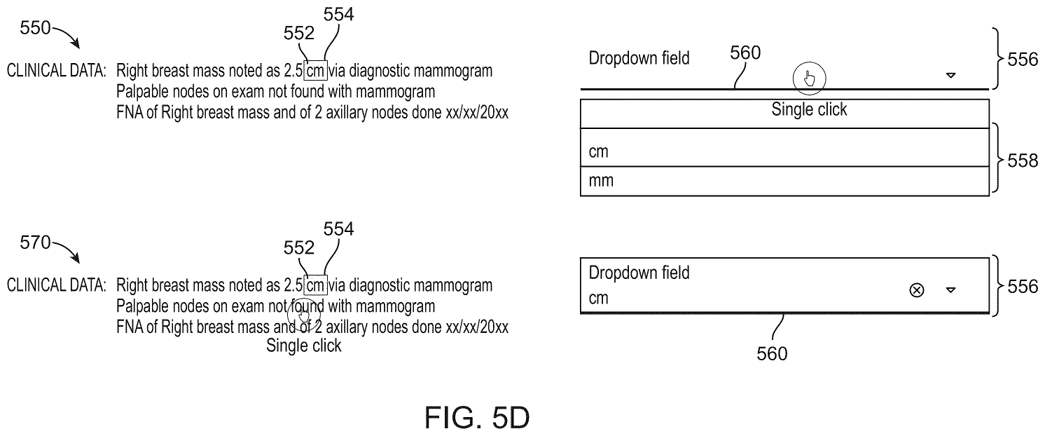

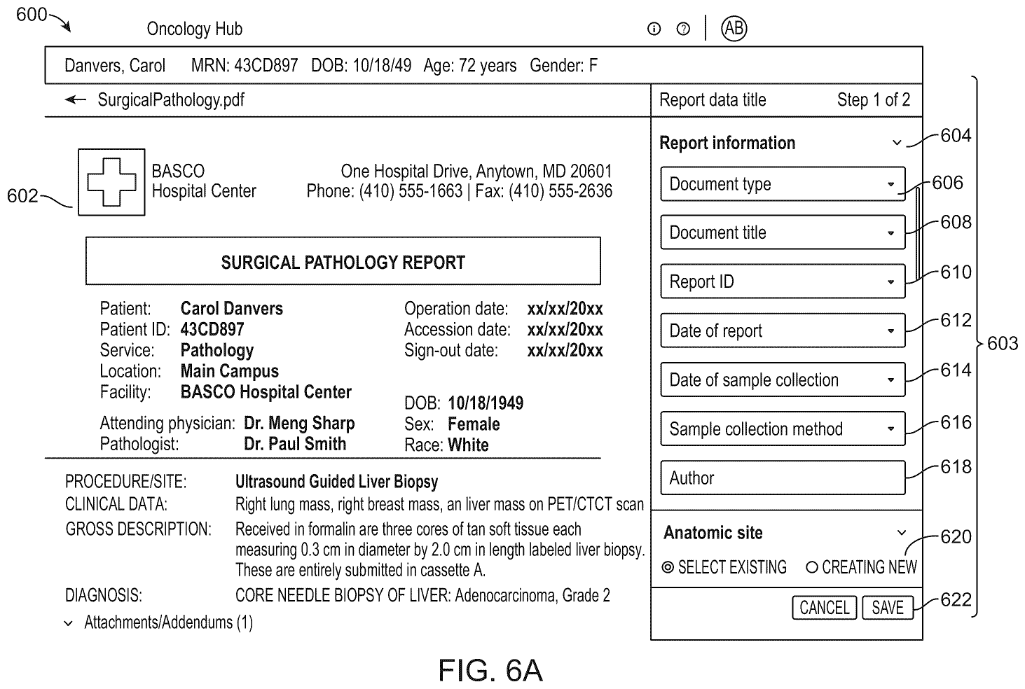

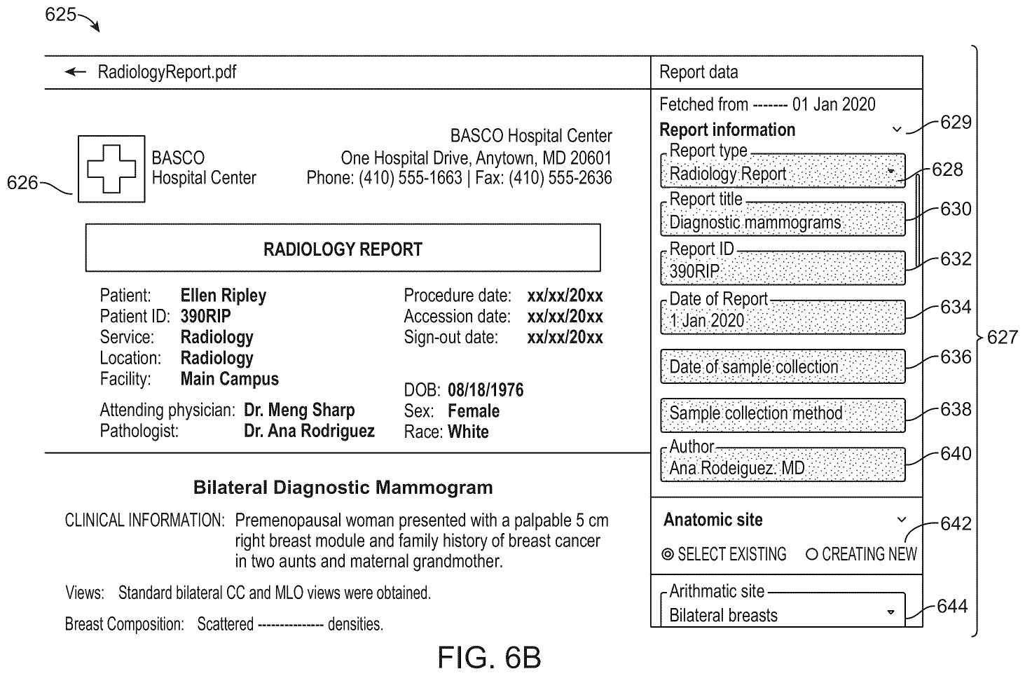

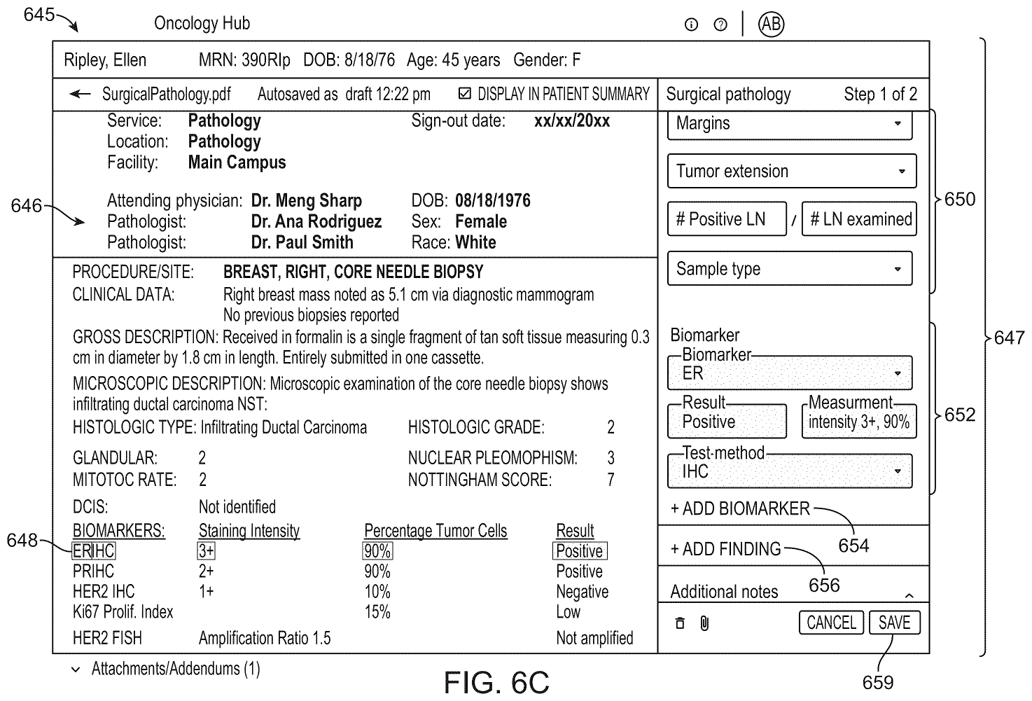

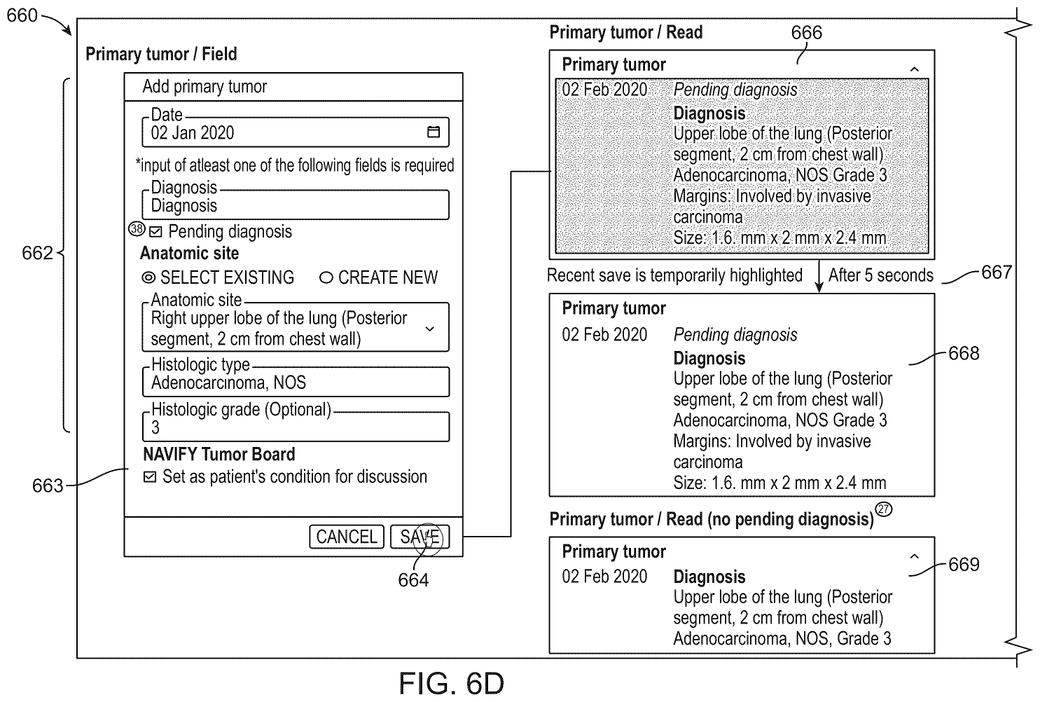

FIGS. 4A-6D illustrate examples of interfaces for managing data from unstructured reports. FIGS. 4A-4C illustrate examples of document abstraction interfaces for importing information from a report file. FIGS. 5A-5D illustrate examples of operations for extracting data from a report using an abstraction interface. FIGS. 6A-6D illustrate different examples of interfaces for extracting fields from reports.

1. Extracting Data from a Report File

In addition to manual entry of data, portal 220 also allows a user to import a document file 244 (e.g., a pathology report, a doctor note, etc.) from patient data sources 240, where data abstraction module 232 can exact various structured medical data from the document file. FIG. 4A, FIG. 4B, FIG. 4C illustrate examples of a document abstraction interface 400 that can be part of portal 220.

FIG. 4A illustrates a document abstraction interface 400 which can be used to guide a user to confirm or update data extracted from a document. As shown in FIG. 4A, document abstraction interface 400 includes a document directory 402, a document browser 404, and an extracted medical data section 406. Document directory 402 can show a list of selectable icons, including icon 407, which represent documents to be selected (or a document that has been selected) to perform medical data extraction and abstraction operations. Moreover, document browser 404 can display the selected document. As described below, document abstraction interface 400 can highlight portions of the document from which medical data are extracted from document browser 404, which allows the user to track the source of the extracted medical data. Extracted medical data section 406 can include a report page 408 and a results page 410. Report page 408 can include a list of metadata extracted from the selected document including, for example, document name 408a, date of report 408b, and document type 408c. Results page 410 includes a set of fields corresponding to a set of categories of data that are to be extracted from the selected document or entered by the user. In some examples, results page 410 can be part of a patient summary as described in FIG. 3A-FIG. 3H.

As described above, the set of fields included in the results page 410 can be defined based on master structured data list (SDL) 246, which data abstraction module 232 can select based on document type 408c. FIG. 4B and FIG. 4C illustrate examples of categories of data to be extracted for different document categories. FIG. 4B illustrates an example results page 411 for a pathology report that provides information about a diagnosis of a cancer. As shown in FIG. 4B, various categories of data can be extracted from a pathology report including diagnostic information 412, staging information 414, and additional notes 416. In addition, diagnostic information 412 can include various fields such as, for example, tumor site information 412a, histologic type 412b, histologic grade 412c, biomarker information 412d, etc., whereas staging information 414 can include various fields to describe the stage of a tumor. In addition, FIG. 4C illustrates an example results page 420 for a cytology report that provides information about the examination of cells from the body of patient. As shown in FIG. 4C, various categories of data can be extracted from a cytology report such as tumor site information 420a and biomarker information 420b. The categories of data shown in FIG. 4B can be defined based on an SDL 246 selected by data abstraction module 232 based on document type 408c of a selected document indicating that the document is a pathology report, whereas categories of data shown in FIG. 4B can be defined based on an SDL 246 selected by data abstraction module 232 based on document type 408c of a selected document indicating that the document is a cytology report.

2. Extracting Results

FIG. 5A, FIG. 5B, FIG. 5C, and FIG. 5D illustrate example operations of document abstraction interface 400 on a pathology report. Document abstraction interface 400 can be used to guide a user to confirm data types for data to be integrated into the unified patient database, such as in fields automatically populated using machine learning. Referring to FIG. data abstraction module 232 can parse the text strings of the selected document (e.g., obtained from an optical character recognition (OCR) processing of the document) and detect text strings that contain data to be extracted, including metadata, data, and various categories of medical data. Data abstraction module 232 can then populate the corresponding fields in report page 408 and results page 410 with the extracted data. Data abstraction module 232 can also cause document browser 404 to display highlight markings, such as highlight markings 502, 504, 506, 508, 510, and 512. Highlight marking 502 can correspond to text indicating document type 408c (e.g., a pathology report), whereas highlight marking 504 can correspond to text indicating a date of the report, both of which can be extracted from the metadata of the pathology report. Fields 520 (report date) and 522 (document type) of results page 420 are then populated with, respectively, the report date and the extracted document type 408c.

In addition, highlight marking 506 can correspond to text describing the procedure involved (e.g., lumpectomy on the right breast), highlight marking 508 can correspond to texts describing the clinical data (e.g., a right breast mass of 2.5 cm is noted via diagnostic mammogram, fine needle aspiration (FNA) of the right breast mass is conducted), highlight marking 510 can correspond to texts describing the right breast mass (e.g., a single fragment of soft tissue received in formalin), whereas highlight marking 512 can correspond to details of a microscopic examination of the right breast mass (e.g., a tumor size of 1.9×1.6×1.4 cm). Fields 524 (e.g., procedure label), 526 (e.g., clinical data label), and 528 (tumor size label) of results page 420 are then populated with, respectively, the texts highlighted by highlight markings 506, 508, and 510. Additional display effects can also be provided to show linkage between fields and the highlighted portions of the document. For example, in FIG. 5A, based on a user selection of field 524, highlight marking 506 can be encircled with a line boundary, whereas the line of field 524 is also emphasized, to indicate correspondence between field 524 and the data covered by highlight marking 506. After the user confirms the populated data and activates publish button 529, data access module 236 can release the data to unified patient database 204.

Data abstraction module 232 can detect text containing medical data and extract the medical data from the text based on various techniques. For example, the detection can be based on a natural language processing (NLP) operation, a rule-based extraction operation, etc., on the text included in the document file. As another example, data abstraction module 232 can detect a select-and-drag action on the document via document browser 404, and the detection can be based on the text selected by the user. After detecting the text strings that contain medical data, data abstraction module 232 can determine the data categories and their associated data values of the medical data, whereas enrichment module 234 can convert the data values to standardized and/or normalized values, or provide options including normalized/standardized values to be chosen by the user. The NLP and rules can be obtained from a training operation based on other medical documents including tags of the data categories as ground truth. For example, the documents used for training may include a sequence of texts “breast, right, lumpectomy” tagged as procedure, which allows data abstraction module 232 to determine that those texts also refer to procedure in the document shown in FIG. 5A. As another example, the documents used for training may include a sequence of text “total size of tumor” followed by another sequence of text noting the size of the tumor. This allows data abstraction module 232 to determine that the sequence of text “1.9×1.6×1.4 cm”, with highlight marking 512, represents the size of a tumor. Enrichment module 234 can then convert the data values to standardized and/or normalized values if needed. For example, if the sequence of texts under highlight marking 512 is “1.9×1.6×1.4 m,” enrichment module 234 may determine that the unit (meters) is not the standard unit and may replace the unit with another unit that is established as the standard unit (e.g., centimeters (cm), millimeters (mm), etc.).

Data collection module 230 can then populate the fields in the results page 410 with the extracted and/or normalized values of the corresponding data categories. In some examples, the population of the fields can be automatic based on a mapping between the data categories and the fields defined in SDL 246. In some examples, the population of the fields can be based on user's selection.

FIG. 5B illustrates an example sequence of operations on document abstraction interface 400 to select texts from document browser 404. Referring to FIG. 5B, operation 530 starts with displaying the document in document browser 404. In operation 532, document browser 404 can receive a select-and-drag action to select part of the document to perform the data abstraction operation, and highlight 533 is displayed to show the extent of the select and drag action and the part of the document being selected at a given a point. In operation 534, document browser 404 can receive a click action from the user, which indicates that select-and-drag action completes and the part of the document selected is confirmed. Document browser 404 can then display a boundary 535 around highlight 533 to indicate that selected text is to be processed by data abstraction module 232 to extract medical data. In operation 536, field 526 of results page 410 can receive a click action from the user, which indicates highlight 533 is mapped to field 526, and the medical data extracted from the part of the document under highlight 533 populates field 526. Document abstraction interface 400 can also display a line 537 in field 526 to indicate that the field is being selected to map to highlight 533. In operation 538, after the population completes, document browser 404 can remove boundary 535 from highlight 533 and line 537 from field 526. The display of boundary 535 and line 537 allow the user to easily visualize which highlighted portion of the document is mapped to which field in results page 410 when the user determines the mapping, which can help the user keep track of the mapping decisions and reduce mapping mistakes, especially in a case where multiple parts of the document are mapped to multiple fields as shown in FIG. 5A.

FIG. 5C illustrates examples of operations on document abstraction interface 400 after the texts in highlighted portion of the document are mapped to the fields in results page 410, to help the user track the source of data in the fields. As shown in FIG. 5C, in operation 540, document abstraction interface 400 detects a click action on field 526. Document abstraction interface 400 can display line 537 in field 526 upon detecting the click action. Moreover, document browser 404 can also automatically scroll to highlight 533 and show boundary 535 around highlight 533, to indicate that the text in field 526 comes from highlight 533. Moreover, in operation 542, document abstraction interface 400 detects a click action on highlight 533. Document abstraction interface 400 can display boundary 535 around highlight 533 upon detecting the click action. Moreover, extracted medical data section 406 can also automatically scroll results page 410 to field 526, also to indicate that the text in field 526 comes from highlight 533.

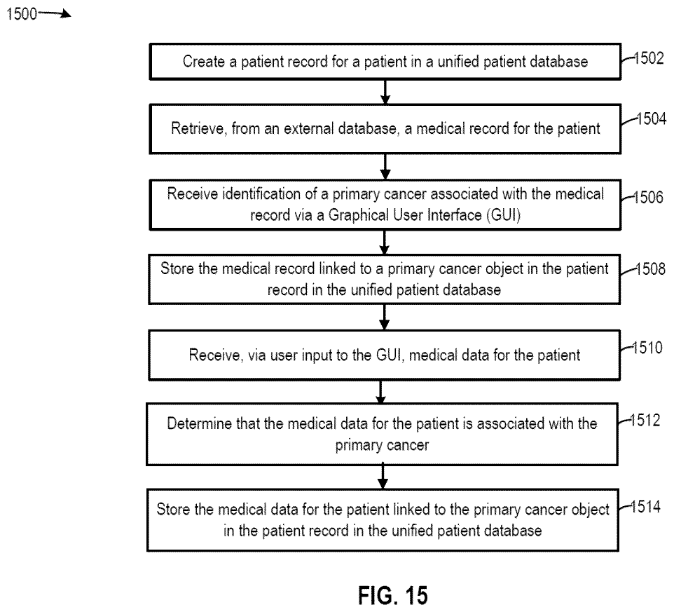

In some examples, data abstraction module 232 can automatically detect texts that may include medical data and extract the medical data from the texts, as further described below with respect to FIG. 15. Enrichment module 234 can determine one or more candidate data values for the extracted medical data for a particular field, based on SDL 246. Document abstraction interface 400 can then provide the candidate data values as options to be selected by the user for the field.

FIG. 5D illustrates a sequence of operations on document abstraction interface 400 involving automatic detection of texts. Document abstraction interface 400 can guide a user to provide or confirm information for use in populating the unified patient database with structured data. As shown in FIG. 5D, in operation 550, data abstraction module 232 detects the text “cm” (centimeters) and causes document browser 404 to display a highlight 552 and a boundary 554 over the text “cm” to indicate that data abstraction module 232 has processed the text. As a result of the processing, field 556 of results page 410 can show a drop-down menu 558 including two candidate values, “cm” and “mm” (millimeters), to be chosen by the user. Document abstraction interface 400 can display a line 560 in field 556 to indicate that the field is mapped to text under highlight 552. In operation 570, document abstraction interface 400 can receive the selection of the candidate value “cm” to populate field 556.

In addition, referring back to FIG. 2, medical data processing system 200 can support an oncology workflow application 222. Oncology workflow application 222 can determine what data to be collected by medical data processing system 200 to support an oncology workflow, which in turn can determine the fields displayed in results page 410 and the categories of data to be received. Moreover, as to be described below, oncology workflow application 222 can perform analysis on the collected medical data and generate analysis results 224.

3. Extracting Data from Reports

FIGS. 6A-6D illustrate additional examples of interface views for extraction and ingestion of data from unstructured reports, according to some embodiments. As shown in FIG. 6A and FIG. 6B, different types of reports are associated with different fields, which can be automatically filled by the system using machine-learning, filled in by a user via the side-by-side view showing both the fields and the report, or a combination of the two.

These reports can come from external systems such as an EMR. Some of the information used to ultimately generate the patient journey interfaces of FIGS. 9A-9E and the patient summary interfaces of FIGS. 8A-8B may come in a structured form from the EMR. Other times, the information is embedded in reports. Information that is embedded in these reports may be unavailable for visualization or analytics because it is not in a structured field. Using the interfaces of FIGS. 6A-6D, the user is shown a list of data fields, allowing the user to enter information in this structured data set. The user can manually enter some of the information while viewing the report, the information can be automatically used to populate fields when ingested in structured form, and/or machine learning is used to scan the document and match up information with a corresponding field.

All the information that comes from these different sources can be consolidated in a single place, i.e., the unified patient database. The data can come from an external source such as an EMR, the data can be manually entered, and/or machine learning such as NLP is used to suggest values, which may be presented to the user for confirmation. All this data is consolidated and enriched within medical data processing system 200.

FIG. 6A shows an interface view 600 including a report 602 side-by-side with a data entry panel 603. The data entry panel 603 includes a set of fields 604-620 that are identified by the system based on an identified type of the report, which can be stored in the report itself, e.g., document type 606. As shown in FIG. 6A, the report 602 is a surgical pathology report, which is associated with a particular set of fields corresponding to surgical pathology. As shown in the example of FIG. 6A, these fields are accessible via a drop-down 604 labeled report information. Other selection mechanisms can be used besides drop-down lists. The fields include document type 606, document title 608, report ID 610, date of report 6012, date of sample collection 614, sample collection method 616, author 618, and anatomic site 620. As described above with respect to FIG. 3G and FIG. 3H, each of these fields can correspond to a data category or data type used to organize and manage the patient data. Based on the fields, the data provided can be stored to corresponding data objects in a data graph. This can also include a data object for the report itself. Examples of such data objects are depicted in, and described below with respect to, FIGS. 12 and 13.

In the example interface view 600 depicted in FIG. 6A, the fields are configured to accept user input via interface elements including drop-down menus 606-616, a text entry field 618, and radio buttons 620. The interface view 600 displays the report 602 side-by-side with the data entry panel 603, so that the user can easily enter information to fill the fields while viewing the report. For example, the drop-down 606 may be populated with each possible type of report which has been previously configured for the system (e.g., radiology reports, pathology reports, etc.). The user can click on the drop-down 606, view the possible types of reports, and select surgical pathology report, which will then be used to populate a corresponding object on the back-end.

Once a user has entered information, the save button 622 may be activated, and, responsive to detecting user interaction with the save button 622, the entered data is saved to the unified patient database 204.

FIG. 6B shows an interface view 625 including a report 626 side-by-side with a data entry panel 627. The data entry panel 627 includes a set of fields 628-644. The fields may be identified by the system based on an identified type of the report. For example, reports for an MRI may be expected to have certain fields, and reports for a mammogram may be expected to have other fields. As noted above, fields for a given report can be identified based on a master structured data list (SDL) 246 that defines a list of data categories for a document type of document file 244.

In the example depicted in FIG. 6B, information has been retrieved from an external system such as an EMR including structured data. Some information is available in the EMR or other external system in a structured form already. This information can be analyzed and associated with a report (e.g., by matching report metadata to the structured data when retrieving the data from the EMR). The interface can include an indication that data corresponding to certain fields were received from the EMR and a given report is tied to these fields. In some implementations, data tied to the reports via information retrieved from a trusted source such as an EMR may be locked for editing, but the user can fill in missing pieces of information. The UI shown in FIG. 6B facilitates augmenting or enriching the data set retrieved from the external system by allowing the user to add missing information to be incorporated into medical data processing system 200.

As shown in FIG. 6B, the report 626 is a radiology report, which is associated with a particular set of fields corresponding to radiology. As shown in FIG. 6B, these fields are accessible for viewing via a drop-down 629 labeled report information. The fields include report type 628, report title 630, report ID 632, date of report 634, date of sample collection 636, sample collection method 638, author 640, and anatomic site 644.

In the example depicted in FIG. 6B, fields 628-640 are highlighted. A particular color may be used to highlight the fields and indicate that the system has retrieved the data populating these fields from an EMR or another external database. Such fields may be locked for user editing. Field 644 is depicted in white, which means that it should be manually filled in by a user. Assigning a site is a diagnostic task that may be best suited to a user such as a doctor. The user can select the radio button 642 for either select existing or create new. In the example depicted in FIG. 6B, select existing has been selected, and an anatomic site drop-down menu is displayed which a user can interact with to select an existing anatomic site. Alternatively, the user can select the create new button and a text entry field will be displayed for entering a name for the new anatomic site.

FIG. 6C shows an interface view 645 including a report 646 side-by-side with a data entry panel 647. The data entry panel 647 includes fields that are identified by the system based on the report 646. As shown in FIG. 6C, these fields are accessible via a drop-down 604 labeled report information. A first set of fields 650, 654, 656, and 658 are configured to be filled via user input (e.g., as described above with respect to FIGS. 6A and 6B). Once a user has entered information, the save button 659 may be activated, and, responsive to detecting user interaction with the save button 659, the entered data is saved to the unified patient database 204.

In the example depicted in FIG. 6C, a second set of fields 652 is depicted with highlighting. The highlighting may be in a different color than used to highlight the fields shown in FIG. 6C, to indicate a different status for the data populating these fields. The highlighted fields 652 correspond to fields 648 highlighted in the report 646. These are fields suggested using machine-learning, which the user can review and confirm or change. In some implementations, the fields are configured to display data that is automatically extracted from the report 646. One or more machine-learning models including optical character recognition (OCR) and natural language processing (NLP) models can be used to identify text data from the report, analyze the report, and identify data that corresponds to certain fields. Medical data processing system 200 may utilize a model which has been trained on labeled data identifying different terms as associated with a given predetermined field. In the example shown in FIG. 6C, the biomarker has been automatically detected by the system. Medical data processing system 200 can populate data elements that are detected using machine learning. The user can be prompted to confirm via the interface, and the user may in some case modify the data elements populating a given field. Over time, medical data processing system 200 can learn and update the machine learning models used to detect data. Using these techniques, the system can provide recommendations in order to reduce the data entry burden on the user. Techniques for applying machine-learning to extract and categorize medical data are described in further detail in PCT Publication WO 2021/046536, titled “Automated Information Extraction And Enrichment In Pathology Report Using Natural Language Processing,” filed Sep. 8, 2020, which is incorporated by reference herein.

FIG. 6D shows a set of interface elements depicting a data entry workflow 660 as can be performed using interfaces such as those depicted in FIGS. 6A-6C. The interface elements depicted in FIG. 6D include interface element 662 for filling in primary tumor information, interface elements 666, 668, and 669 for reading primary tumor information, and interface elements 670 and 674 for editing primary tumor information.

In FIG. 6D, the interface element 662 for filling in primary tumor information includes a set of fields for accepting user input of information associated with a primary tumor. The fields include an interface element for adding information about an anatomic site (e.g., right upper lobe of lung, which is selected from a drop-down menu when the “select existing” radio button is selected). The fields further include a histologic type and histologic grade. A user can fill in the diagnostic information. The interface element 662 further includes a user-selectable check box 663 that can be checked to set the diagnosed primary tumor as patient's condition for discussion. As indicated by the cursor and highlighting on the save button 664, a user can click on the save button 664 to save the entered information to the unified patient database 204.

FIG. 6D, the interface elements 666, 668, and 669 for reading primary tumor information display the information that was entered via interface element 662. In interface element 666, recently entered information is temporarily highlighted. In interface element 668, after 5 seconds (or another suitable timeframe), the entered information is no longer highlighted. In interface elements 666 and 668, the diagnosis is flagged as a pending diagnosis. In interface element 669, the primary tumor is not marked as a pending diagnosis, and the pending diagnosis flag is not present.

FIG. 6D, the interface elements 670 and 674 are for editing primary tumor information. A user may interact with an interface element for reading primary tumor information such as interface element 666. As shown on interface element 670, a cursor is clicking the highlighted primary tumor diagnosis. The highlight remains until editing is complete. On click, the color goes to a focused state and an edit drawer 674 is opened. The edit drawer 674 is an interface element such as a modal that opens on detecting user interaction such as a click. The edit drawer 674 includes fields for accepting user input to edit the information previously input (e.g., via interface element 662). The components of the drawer 674 include data entry fields that can be used to edit fields such as date, diagnosis, pending diagnosis 676, anatomic site 680, and histologic type 682. The edit drawer 674 further includes radio buttons 678 to select existing or create new anatomic sites. These interface elements can be used to retrieve data to update the data stored to unified patient database 204. The retrieved data can additionally or alternatively be used to train the machine learning models used for automated data extraction from documents (e.g., if medical data processing system identifies that a field was incorrectly populated by the model based on user modification, this can be used to update training data for the model).

C. Interfaces for Reconciling Unmapped Data

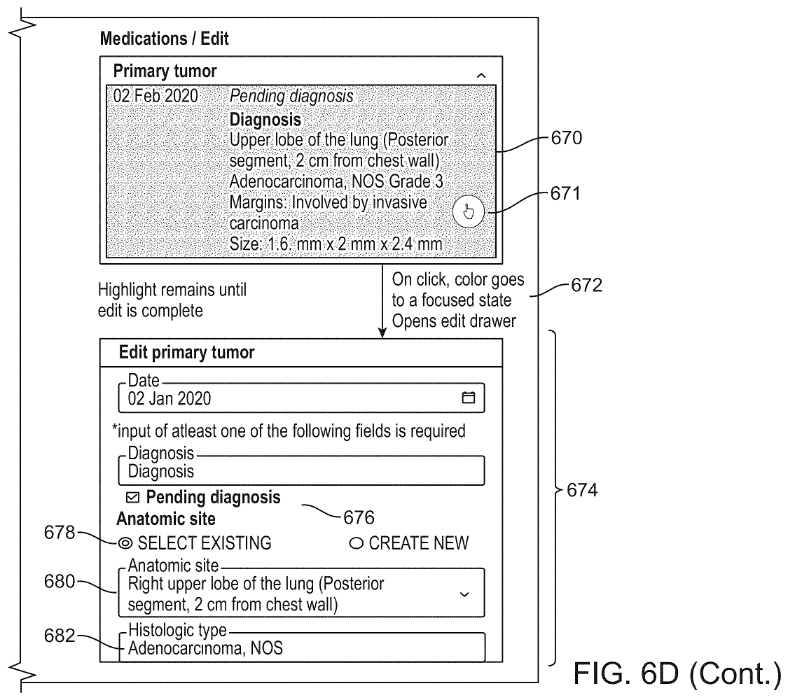

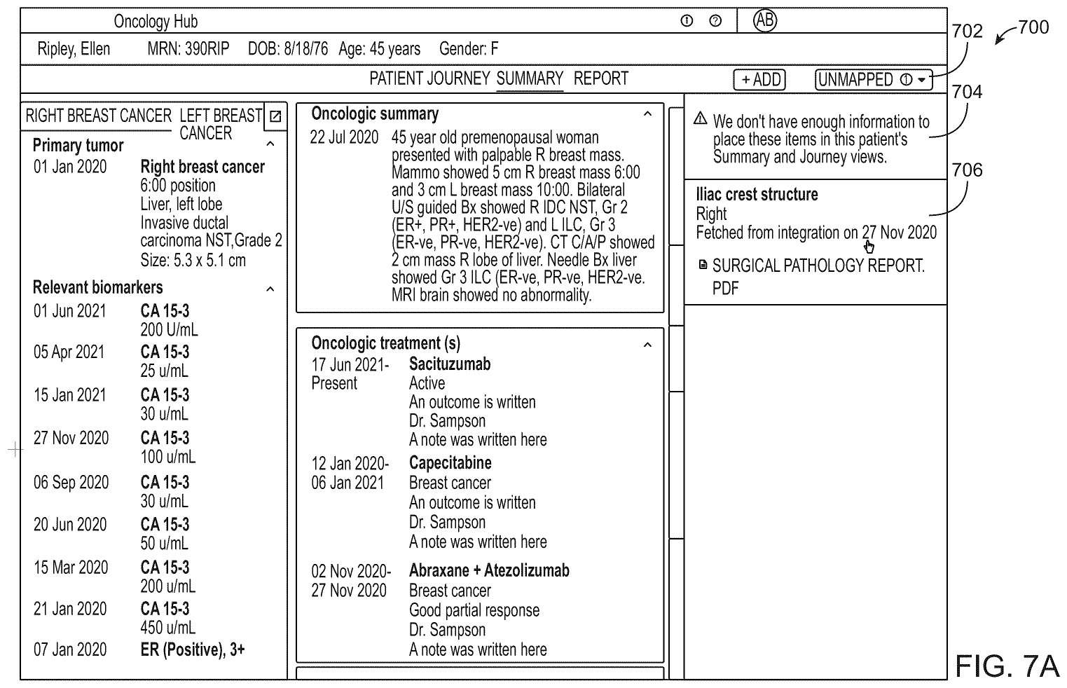

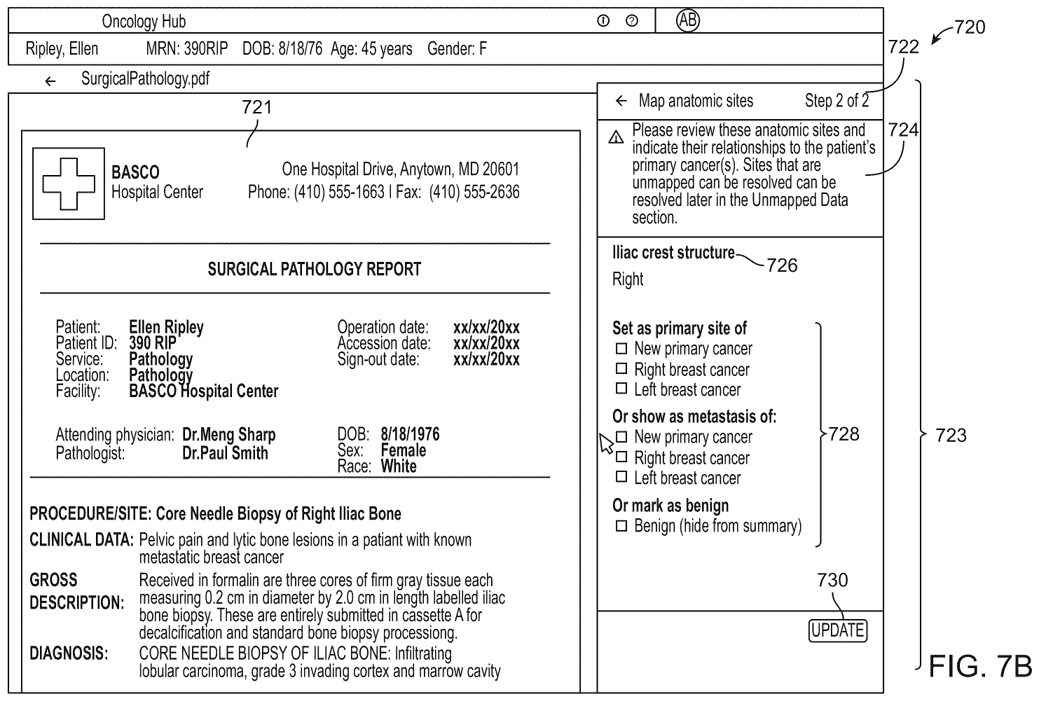

FIGS. 7A and 7B illustrate examples of interface views for reconciling unmapped data, according to some embodiments. Reconciliation may be initiated if data is not mapped to a data field that is deemed necessary, such as association of a cancer mass with a primary cancer (e.g., as a new primary cancer or as a metastasis of another primary cancer). The interfaces depicted in FIGS. 7A and 7B can be used to manage such a reconciliation process prompting the user for necessary information, even after a record has been stored for the cancer mass at issue. For instance, missing information may be flagged in the unified patient database 204 for reconciliation, prompting the workflow described below.

For data that comes from source systems such as an EMR, for some data elements, the relationship between data elements could be missing. In one example, the patient has two primary cancers and a metastatic site. The primary cancers and the metastatic site have been retrieved from a report via the EMR, but a primary associated with that metastatic site is unknown. The clinician may know information not retrieved from the EMR. For such use cases, the system has a reconciliation of unmapped data function.

In reconciliation, certain data has been abstracted but the system still needs to determine where in the UI the data belongs, e.g., to which primary condition the data should be mapped. The reconciliation UI can prompt the user to provide input to associate that particular anatomic site, for example, with the right primary cancer. For a given primary cancer, certain fields can be associated with the primary cancer. The reconciliation UI prompts the user to associate different types of information such as the primary site with related observations such as histology, the biomarkers, the stage, and the metastatic site uniquely to a primary cancer or other data elements. The reconciliation UI may also be used to map certain medical interventions such as oncology treatments or non-oncology surgical history, or certain drugs as antineoplastic or non-cancer, for example.

In some cases, an external system such as an EMR provides information indicating a primary cancer and where this primary cancer has metastasized. In this case, the association is known, and additional work may not be needed. In other cases, in which reconciliation is needed, the external system either is not capturing the association or is not sending that information to medical data processing system 200. If such an association is needed, Medical data processing system 200 may use the reconciliation process to determine where in an interface such as the patient summary or patient journey view to show that metastasis (e.g., against the right breast or the left breast). To be able to present information in a clinically accurate way, the reconciliation process enables the user to provide guidance on where to show this association, which affects the data mappings applied in unified patient database 204.

In some instances, reconciliation can be triggered when an external database such as an EMR sends data pertaining to a particular site, but information indicating other sites with which to associate that site is missing. Using the reconciliation interface, a user can provide information to associate a site with a particular cancer, and after an update, the site will start showing up in association with the correct primary cancer in the interface views and the unified patient database. In other instances, reports may be received from an external database without any structured information, in which case multiple granular details may be missing. Such details can be provided by the user via the interfaces such as that depicted in FIG. 7A.

FIG. 7A shows an interface summary view 700 including data reconciliation elements 702, 704, and 706. In some implementations, in the interface summary view 700, a data reconciliation element 702, such as a button or drop-down menu, is provided for interacting with unmapped data for reconciliation. At the top right of the screen, this data reconciliation element 702, an “unmapped” button, allows the user to open unreconciled items which are not related to any cancer, or otherwise missing mapping information. The user can provide data specifying the missing relationships are and save the updated data. When the user reconciles this data, this data will then start appearing in the portal 220.

User interaction with reconciliation element 702 may trigger display of data reconciliation elements 704 and 706. Data reconciliation element 704 includes a notification, displayed in a conspicuous manner (e.g., highlighted and displayed with a warning sign). In the example depicted in FIG. 7A, the notification displayed in data reconciliation element 704 states “We don't have enough information to place these items in the Patient Summary and Journey views.” Information about the item requiring reconciliation is displayed in the data reconciliation element 706. In this example, a cancer mass, iliac crest structure, is missing information necessary to add it to the patient summary and journey views. The data reconciliation element 706 further provides additional information about the cancer mass—“right” and “fetched from integration on 27 Nov. 2020.” Such information may be retrieved from the unified patient database according to the data types of the mappings therein (e.g., the fetched from integration date may be based on a timestamp and the right side may be based on a position data type). On user interaction with data reconciliation element 706, the interface can transition to the interface view depicted in 7B for reconciliation.

FIG. 7B shows an interface view 720 for data reconciliation. In some implementations, in the interface summary view 720 includes a report 721 and a drawer 723 (e.g., a modal with elements for accepting user input) for accepting data for reconciliation. Within the drawer 723 is included a heading 722, labeled “map anatomic sites.” Drawer 723 indicates that the missing information to be reconciled is to associate the iliac crest structure 726 with a primary cancer or metastasis, or mark it as benign. Drawer 723 also includes an alert 724, similar to the data reconciliation element 704 described above with respect to FIG. 7A. The drawer 723 of the interface view 720 further includes a set of check boxes that a user can use to associate the iliac crest structure 726 with a particularly primary cancer or metastasis, or mark the iliac crest structure 726 as benign. In some implementations, the unified patent database stores a patient record with objects corresponding to different cancer sites. Based on the anatomic site mapping established using the interface view 720, the object for the iliac crest structure 726 can be linked to other objects accordingly. For example, if the iliac crest structure 726 is marked by the user as a metastasis of right breast cancer, the iliac crest structure object will be linked to the right breast cancer object. The received designation of the iliac crest structure as a primary, metastasis, or benign may be stored to the unified patient database in association with a “behavior” data type in a data object for the tumor mass, as further described below with respect to FIG. 12.

As shown, the possible choices include setting as a primary or metastasis of a new primary cancer, which may trigger display of additional interface elements for establishing a new primary cancer. The possible choices further include setting the iliac crest structure as a primary site. This will cause the iliac crest structure to be stored in the unified patient database as a primary cancer object, which will have its own set of linked objects as shown in FIG. 12. Alternatively, the iliac crest structure is set to a metastasis of pre-established cancers—a right breast cancer or a left breast cancer. This will cause the iliac crest structure to be stored in the unified patient database as an object linked to a type metastatic object and linked to another data object corresponding to the selected primary cancer. Another check box is provided to mark the iliac crest structure 726 as benign, which will cause it to be hidden from the summary. In such an event, the iliac crest structure 726 may be stored in the unified patient database as an object linked to a benign type object and not linked to any objects corresponding to primary cancers. Once the user has selected an association for the cancer, the update button 730 will be activated. The user can interact with the update button to trigger the system to store the provided reconciliation data to the unified patient database 204. The data schema for storing the data objects responsive to the selected anatomic site mapping is described in further detail below in section IV with respect to FIGS. 12 and 13.

D. Patient Portal Interfaces

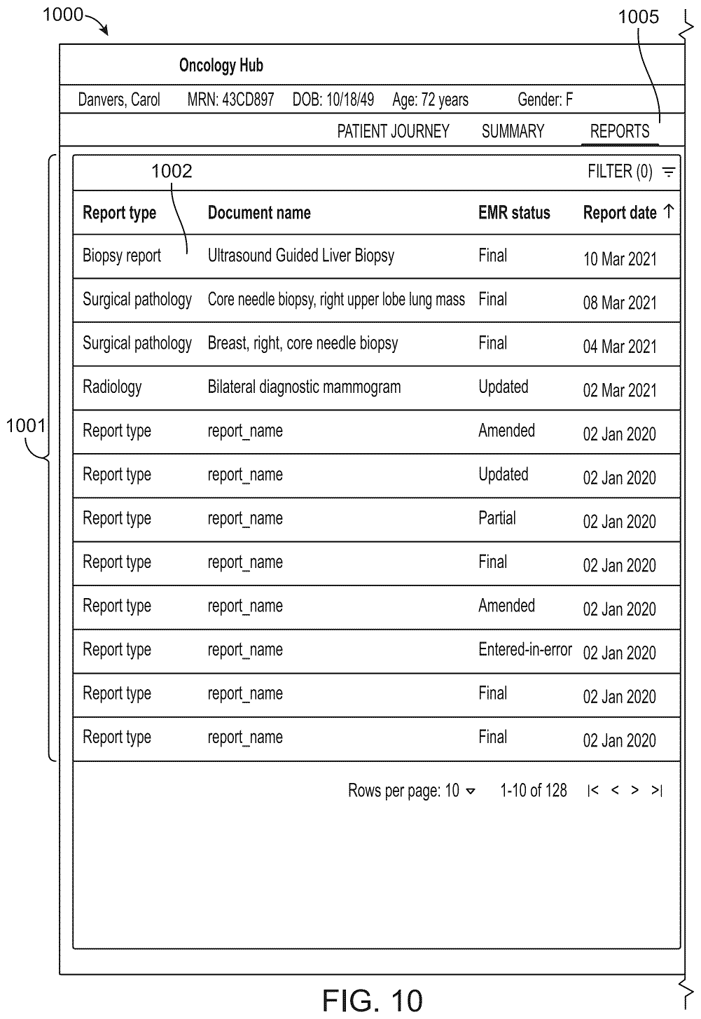

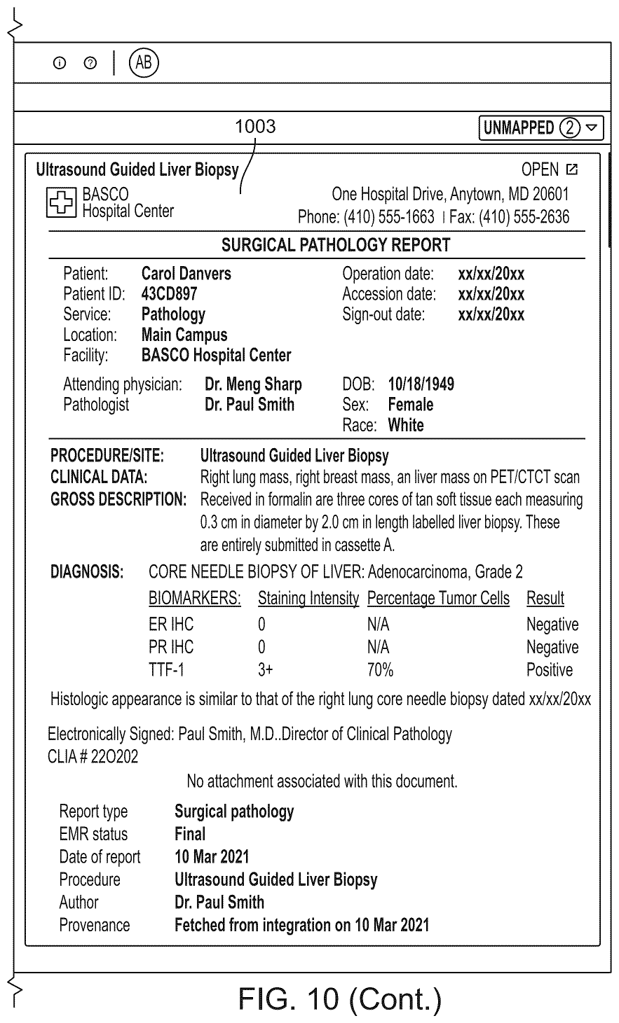

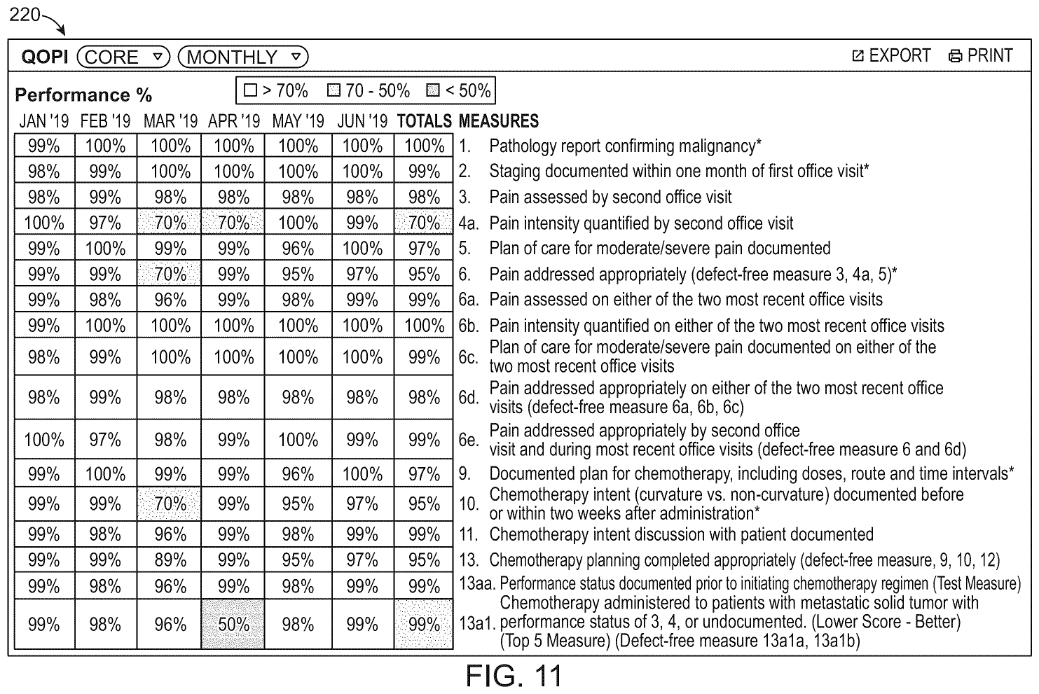

FIG. 8A, FIG. 8B, FIG. 8C, FIG. 9A, FIG. 9B, FIG. 9C, FIG. 9D, FIG. 9E, FIG. 10, and FIG. 11 illustrate examples of portal 220, which provides a centralized view of patient data. Portal 220 can display various interface views, including patient summary interface views as shown in FIGS. 8A-8C, patient journey interface views as shown in FIGS. 9A-9E, reports interface views as shown in FIG. 10, and care quality metric interface views as shown in FIG. 11.

1. Patient Summary Interfaces

FIGS. 8A-8C show examples of patient summary interface views, according to some embodiments. The patient summary interface displays summary data for a patient. The patient summary interface views can be used to display data enabling a user to view detailed information about different primary cancer sites and oncologic summary information, as well as provide a launching pad to perform data input and reconciliation via the portal 220.

Referring to FIG. 8A, portal 220 can show the current diagnosis result of a patient (Adenocarcinoma of the lung), the diagnosis date, notes from the last visit, upcoming visits, and current treatment. Portal 220 can receive structured patient data 202 from unified patient database 204 which are either entered manually via data entry interface 300 or automatically sourced and abstracted from medical reports by data abstraction module 232.

FIG. 8B shows another implementation of a patient summary interface 800 of portal 220. The patient summary interface 800 shows summary information about a particular patient, which can be fetched from the unified patient database 204 for display. A top ribbon 801 can display patient information such as the patient's name, age, date of birth, gender, and an identifier of the patient.

The patient summary interface 800 includes a primary cancer element 802. The primary cancer element 802 includes tabs 803A and 803B corresponding to different primary cancers, breast cancer (in tab 803A) and lung cancer (in tab 803B). In the primary cancer element 802, information about each primary cancer is described, including events, relevant biomarkers, staging, and metastatic sites.

In FIG. 8B, the patient summary interface 800 further includes an oncologic summary element 804, oncologic treatments element 806, and medications element 808, displaying information about each. The patient summary interface includes a patient history element 810, which shows patient history information including medical history, surgical history, family history, and social history.

The patient summary interface 800 also includes user-selectable elements that can be used to navigate to other interface views. The patient journey element 811 can be selected to transition to the patient journey view as shown in FIGS. 9A-9E. The reports element 812 can be selected to transition to the reports view 1000 as shown in FIG. 10. The unmapped data element 813 can be selected to transition to the reconciliation view depicted in FIG. 7B. The add element 814 can be selected to transition to the views for adding data (e.g., directly into the portal 220 or by uploading a report). A summary element 815 is also included and can be used to transition to the patient summary view from other views. Thus, the patient summary view can be used to transition to various views of the portal 220. Primary information element 805 can be selected to cause a modal to be displayed, overlaid on the patient summary view 800, and including additional information about one or more primary cancers, as shown in FIG. 8C. Based on the selected view or mode, data is retrieved from unified patient database 204 according to the mappings of data connections and types therein.

Referring to FIG. 8C, an example of a patient summary view 820 with two modals 822 and 824 corresponding to two primary cancers is shown. Responsive to detecting user interaction with the primary information element 805, in this example, modals for both of the primary cancers associated with the patient are displayed. Modal 822 (e.g., a first modal) is for the right breast cancer, and includes information about the right breast primary cancer including a set of relevant biomarkers with timestamps. Modal 824 (e.g., a second modal) is for the left breast cancer, and includes information about the left breast primary cancer including a set of relevant biomarkers with timestamps.

The first modal and the second modal are displayed side-by-side in the graphical user interface. Advantageously, the side-by-side view allows the user to view more detailed information about multiple primary cancers at once, without navigating away from the summary interface screen. Further, since each modal corresponds to a different primary site, the data can be retrieved efficiently for each site, so that the side-by-side analysis can be provided. This organization of the database (e.g., the data schema described in section IV) enables such retrieval of data and visualization via the graphical interface.

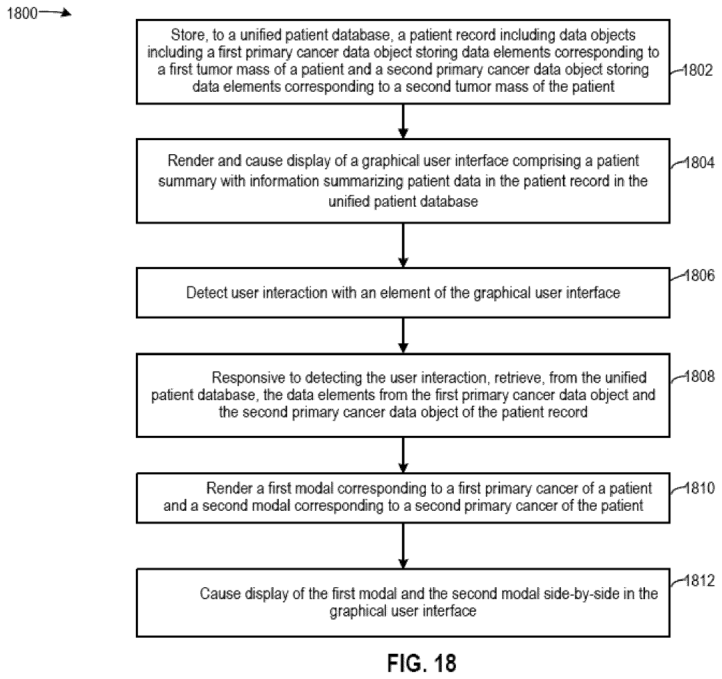

In some implementations, the unified patient database stores a data object corresponding to the right breast primary cancer and a data object corresponding to the left breast primary cancer. These data objects are linked to various other data objects, which are timestamped and descriptive of different events associated with the primary cancers. For example, the right breast primary cancer object is linked to one or more biomarker data objects and the left breast primary cancer object is linked to one or more biomarker data objects. Responsive to detecting user interaction with the primary information element 805, the system queries the unified patient database to identify the right breast primary cancer data object and the left breast primary cancer object. Objects linked to each primary cancer data object are identified based upon mappings in the unified patient database between the identified primary cancer data objects and respective child data objects. Information is retrieved in association with the identified linked objects. The identified linked objects are used to populate the modals 822 and 824 with the retrieved information, as further described below with respect to the method 1800 of FIG. 18.

The right breast cancer and the left breast cancer may be different cancers located at different parts of the body which are unrelated. The patient summary view 820 can be used to show information associated with each of the primary cancers, with the information that is different about each primary shown side-by-side. Using the interface view 820, a clinician can observe information about multiple primary cancers, and compare information such as diagnosis, onset date, the location of each primary site, the key biomarkers for this patient, the staging, and any metastasis. This can be achieved using the specialized data schema described herein to organize data according to primary cancer designations, which can be fetched to display the interface view 820 displaying the primaries side-by-side.

2. Patient Journey Interfaces

FIGS. 9A-9E show examples of patient journey interface views, according to some embodiments. The patient journey interface displays data associated with the patient in chronological fashion. Using the patient journey interfaces depicted in FIGS. 9A-9E, the progression of the cancer and available information about the cancer can be viewed in an organized and chronological fashion. The user can click on objects in the timeline and see how the cancer has evolved.

The patient journey interface views can show patient information from the point of suspicion of cancer to diagnosis, treatment planning, monitoring, survivorship, and so forth. Cancer care is essentially both multidisciplinary multi-institutional. Generally patient information may be scattered across different systems. By extracting and integrating information from reports and other data retrieved from disparate sources, the medical data processing system can build an interinstitutional patient journey, enabling a user to view a holistic patient journey across data points gathered across different service providers and service types.

Once this information is in the patient journey, any other user following the patient journey can see the information depicted in the exact same way. This is advantageous from a care collaboration perspective. Using prior techniques, the over-reliance on medical notes results in different providers often taking away different information about what is happening to a patient based on different note-taking styles. It is therefore difficult, using prior systems, to get a common understanding of what is truly happening with a patient. The patient journey interface illustrated in FIGS. 9A-9E solves these problems and others by allowing any user to see a unified view of the patient's treatment history. The patient journey can be viewed and populated by a cross-function team, such as a radiation oncologist, a medical oncologist, a surgical oncologist, and/or an attending physician. These users will be able to interact with the patient summary UI and be able to see the patient data in a user-friendly, unified way to observe and understand the evolution of patient conditions such as cancer.

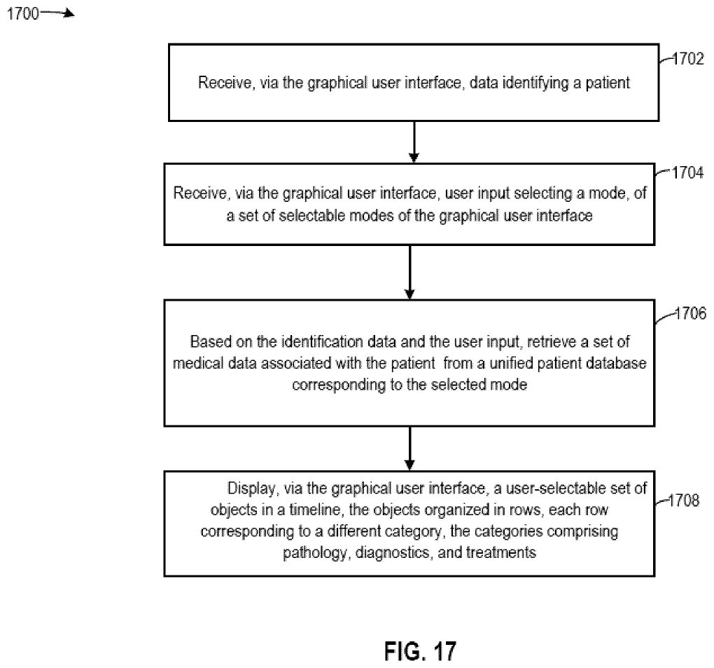

To display the patient journey view, the system may receive, via a graphical user interface, data identifying a patient (e.g., patient ID, name, etc.). Based on the data identifying the patient, the system may retrieve, from a unified patient database, medical data associated with the patient and display, via the graphical user interface, a user-selectable set of objects in a timeline, the objects including a plurality of categories organized in rows, the categories comprising pathology, diagnostics, and treatments, as shown in FIGS. 9A-9E. Techniques for populating the patient journey view are further described below with respect to the method 1700 of FIG. 17.

Referring to FIG. 9A, portal 220 can show a timeline view of the patient journey. The timeline view can show various lab tests and imaging results, as well as diagnosis results provided by oncology workflow module 222, with respect to time.

FIG. 9B is another implementation of a patient journey interface view 900. The patient journey interface view 900 of the portal 220 includes a summary ribbon 902 and an adjustable timeline 908.

The summary ribbon 902 can be a ribbon displayed above the timeline. The summary ribbon 902 can display a subset of the objects flagged as significant and associated information. The user has the ability to bookmark objects to be displayed in the summary ribbon 902. Given the patient journey can be long and include many objects, the summary ribbon 902 is useful for bringing significant objects to the forefront. The user can also remove an object from the bookmark when the object is no longer important, and the object will be removed and disappear from this ribbon. This summary ribbon 902 can serve as a mini journey in itself showing the key objects that have happened with this patient.

The adjustable timeline 908 includes information about the patient's oncological history. The adjustable timeline 908 displays the information in chronological order, with older objects towards the left and newer objects towards the right. The period of time displayed can be controlled with start and end date elements 904 and 905, as well as a scroll bar 906 that is adjustable to select the time window for which objects are displayed in timeline 908.

The information in the timeline 908 is displayed in a set of rows corresponding to different data categories, including events 910, pathology 912, diagnostic imaging and procedures 914, treatments 916, biomarkers 918, and response evaluation 920. For each category, the associated information may be color-coded (e.g., events in orange, pathology in red, etc.). Each row may display information gathered about the patient in the corresponding data category. A given row may include multiple entries at a given time, as shown in FIG. 9B. For example, in events 910, multiple events in January correspond to two different cancers.

The events 910 data category includes events (e.g., a category of displayed objects) corresponding to information about the progression of the cancer itself. For example, events 910 include breast cancer, invasive 922, dated in January 2020. This may correspond to a date when this primary cancer was diagnosed and added to the patient record. If a user clicks on event 922, the system will show additional information about the event 922, as shown in FIGS. 9C and 9D. The events 910 row can show cancer diagnoses for each different cancer that the patient has, as well as the progression of the cancer. For instance, as shown in FIG. 9C, event 922 is the right breast invasive ductal carcinoma on the site at 6 o'clock position. As the user clicks on these different items in the interface view 900, the user will be able to see how the cancer has evolved. For example, when the cancer first started, there were no metastases. The user can scroll through the timeline to see that after one year or two years, if the cancer has metastasized somewhere else, then that will be visible in a particular box. Thus, the patient journey interface view 900 allows a user to see the progression of the cancer over time. This information may be retrieved from tumor mass data objects 1202 and/or cancer condition data objects in the unified patient database according to the data schema depicted in FIG. 12.

The pathology 912 data category includes objects corresponding to pathology reports, displayed chronologically. If there are multiple reports that are associated with a date, the multiple pathology reports can be displayed in a stacked fashion. Pathology reports may be associated with the events 910, e.g., used to diagnose a particular cancer mass. Examples of pathology reports include biopsy reports, cytology reports, genomic reports, surgical excision reports, etc. Via the patient journey interface view 900, the user can drill down into a particular pathology report to discern information such as how much the cancer has spread, what the size is, what the stage is, the key biomarkers that you test from that sample that you obtained, and so forth. This information may be retrieved from diagnostic findings data objects 1205 in the unified patient database according to the data schema depicted in FIG. 12.

The diagnostic imaging and procedures 914 data category includes objects corresponding to diagnostic imaging such as MRIs, CT scans, and so forth. For example, the diagnostic and imaging procedures 914 includes an MRI 924 from 14 Jan. 2020, and so forth, as shown in FIG. 9B. These objects are displayed in a chronological sequence of objects. The objects can link to diagnostic imaging reports such as an MRI report or some lesions. A clinician looking at this information should be able to see that, on a given date in the timeline, there was an MRI done for this patient, and drill down to view the results of that MRI by opening the report. For example, if the user clicks on the MRI of 14 Jan. 2020 (924), the report can open directly from the patient journey interface view 900. Advantageously, the user need not navigate to another system to look for the report, which would be required without the techniques of the present disclosure. This information may be retrieved from diagnostic findings data objects 1205 in the unified patient database according to the data schema depicted in FIG. 12.

The treatments 916 data category includes objects corresponding to treatments given to the patient. As seen in FIG. 9B, the treatments may span over several months. This information may be retrieved from treatment data objects 1208 in the unified patient database according to the data schema depicted in FIG. 12.

The biomarkers 918 data category includes objects corresponding to biomarkers associated with the patient. This can include genomic markers, diagnostic markers, prognostic markers, therapeutic markers, and so forth. These biomarkers may originate from various types of reports, but are handled similarly in the system. For example, biomarkers can come from a cytology report, a genomic report, etc. These various types of biomarker objects are all shown in the biomarkers 918 row. This information may be retrieved from diagnostic findings data objects 1205 (e.g., molecular/biomarker objects) in the unified patient database according to the data schema depicted in FIG. 12.

The response evaluation 920 data category includes objects corresponding to clinician assessments of a patient response. At each step in disease management, clinicians assess the patient's tumor status and clinical condition to determine the effect of a treatment, and decide whether and how to continue the current treatment plan. And determining treatment effectiveness is a complex judgement based on elements of clinical response, radiologic response, molecular response, and serologic response. Patient journey interface view 900 condenses this down to a very telegraphic single icon view on the timeline so clinicians can see it chronologically in line with treatments, scans and other data. For example, a doctor may make note that a patient is given a certain number of cycles of a particular drug, along with radiation therapy, and the patient has partially responded. Using the patient journey view, if at any point the clinician wants to record how this particular cancer is progressing, the user can input an assessment of the response, such as whether it is a partial response, whether the cancer is stable, how the patient is feeling, any adverse events, if the cancer progression is uneventful, and so forth. In some implementations, patient journey is completely read only, except for this one field of response. A key job of an oncologist is to be able to manage toxicities and to monitor a patient's response while on a treatment, therefore response assessment by a clinician is critical in many situations.

In FIG. 9C, a cursor 923 is hovering over breast cancer, invasive element 922. This causes the system to expand the view so that additional text is visible in the breast cancer, invasive element 922—ductal carcinoma, right breast (6:00).

In FIG. 9D, if the user then clicks on breast cancer, invasive element 922, a pop-up 927 will be displayed to show further information such as the date, location, and so forth, as shown in FIG. 9D.

In FIG. 9E, the adjustable timeline 908 has been adjusted (e.g., via the slider) to show a different time window. In FIG. 9E, the time window from 1 Oct. 2020 to 1 Jan. 2021 is shown. Thus, via user interaction with the GUI, the user can scroll around to display different objects in the timeline by moving the slider 906 to view the timeline over a longer time period or zero in on time periods of interest.

In some implementations, a report can be previewed from the patient summary view. The system can detect user interaction with an object displayed in the patient summary view, then identify and retrieve a corresponding report from the unified patient database and display the report via the graphical user interface (e.g., as a popup on the patient summary view). The user can navigate to the reports view for a more detailed view of the reports.

The patient journey view can be used to see how the patient's cancer evolved over time. For example, a first time, the patient has one primary cancer site (e.g., in the example shown in FIG. 9A). At a second time, one primary is still visible in the patient journey view. At a third time, two different primaries can be seen (e.g., in the example shown in FIG. 9B). Thus, in this particular example depicted in FIGS. 9B-9E, two primary cancers, left and right breast cancer, are displayed in the patient journey.

3. Reports View Interface

FIG. 10 shows an example of a reports interface view 1000 according to some embodiments. The reports interface view 1000 includes a list 1001 of reports associated with the patient. One of the listed reports 1002 has been selected, and that report 1003 is displayed on the right hand side. An interface element is also provided that the user can click on to cause the full report to be opened.

In some implementations, the patient journey, summary, and reports tabs at the top (1005) can be used to navigate between the respective interface views. For example, in the patient journey view, the system detects user interaction with the summary tab and transitions to the summary view, displaying oncologic summary data.

4. Quality Care Metric Interfaces

FIG. 11 shows another interface view for displaying quality care metrics. As shown in FIG. 11, portal 220 can show a care quality metric, such as Quality Oncology Practice Initiative (QOPI) with respect to time for different patients. The metrics can be computed based on the structured patient data 202 at different time points.

### IV. Example Schema for Unified Patient Database

FIGS. 12 and 13 show example data schema for use in structuring data stored to the unified patient database. The patient summary and patient journey interfaces described above are enabled by retrieving interconnected data elements associated with a patient, which are timestamped and tied together hierarchically. These data elements are dynamically updated and enriched. This is made possible using a specialized data schema for the unified patient database. FIG. 12 shows examples of different types of data objects connected together in a patient data map. FIG. 13 shows an example of specific data objects that may be stored and modified.

A. Data Schema for Patient Data Elements

FIG. 12 shows an example data schema 1200 for patient data elements according to some embodiments. Using the data schema 1200, the disparate data elements retrieved by medical data processing system 200 are broken down and used to generate discrete data objects (also referred to as data entities). The data objects store various data elements associated with patient data. Relationships are maintained between, and updated for, these data entities. This data schema allows the system to continuously maintain the most current up-to-date picture of the patient, with detailed data elements stored in a structured fashion. In some implementations, the data schema is based on the HL7 FHIR (Fast Healthcare Interoperability Resources) standard, as described in “Welcome to FHIR,” https://www.hl7.org/fhir/(2019)).

In this example, the data schema 1200 includes a set of data objects (pictured in boxes, e.g. tumor mass(es) data object 1202, diagnostic findings data object 1205, etc.). In the unified patient database, for each patient, a patient record can be stored. The patient record includes a network of interconnected data objects, each of which can include a configured set of data elements of data types corresponding to a given data object. Each box depicted in FIG. 12 is a data object, which can be implemented as a resource using the HL7 FHIR Standard. Alternatively, the data objects can be implemented as tables using relational databases.

As shown in FIG. 12, each data object has associated attributes in the form of a set of data types corresponding to that type of data object. For example, the data schema 1200 includes a tumor mass(es) data object 1202, which may store data elements corresponding to data types such as history, anatomic site, site description, and behavior, as shown in FIG. 12. The data types may correspond to the fields shown in the interface views (e.g., fields 606-620 shown in FIG. 6A, fields 628-644 shown in FIG. 6B, etc.).

Each data object such as tumor mass(es) data object 1202, diagnostic findings data object 1205, and patient root data object 1201 represents a clinical data entity. These data objects can be related to each other, which facilitates management of a graph of patient data that is a network of interconnected data objects. For example, a given data object can include information including a data element, such as “colon,” in connection with a corresponding data type characterizing or classifying the data element, such as “site.”

In FIG. 12, the lines 1220 connecting the data objects indicate the relationships between the elements. For example, cancer condition data objects must be linked to a patient data object and one or more tumor mass data objects, and can optionally be linked to one or more oncology treatment data objects. Circles 1222 indicate what can be optional, single solid bars 1224 indicate a one-to-one relationship, v-shaped symbols indicate a one-to-many relationship, and a circle along with a v-shaped symbol (e.g., the middle connector for reports 1204) indicates a zero-or-many relationship. A link (connection) between objects can be specified in various ways within the unified patient database. For example, a master list can be stored for a patient record that identifies each object that is linked to another object. A direct of the link can be specified, e.g., a report from which a tumor mass was created.

Root data object 1201 is a data object for the patient, and can include information such as the patient's name, date of birth, gender, and identifiers. As indicated by the connecting lines 1220, root data object 1201 is connected to various other data objects corresponding to oncological data for that patient. The data objects can be classified in terms of diagnosis, treatment, history, or other suitable categories of data. Each of the other data objects can be tied back to the patient root data object 1201. Information from the patient root data object 1201 may be displayed along the top of the interface views of the portal 220 (e.g., patient ribbon 801 of FIG. 8B). The patient root data object 1201 can be used to identify and traverse the patient data record to identify additional information for display and editing via the portal 220.

Various data objects, corresponding to data types, are connected to the root data object 1201 for the patient. Every data object is related to the patient root data object in some manner. For example, the diagnosis-related data objects 1203 are data objects that are used to describe diagnosis information for the patient. Each of the diagnosis-related data objects 1203 is connected to the patient root data object 1201. The diagnostic findings data object 1205 is a data object corresponding to diagnosis connected to the tumor mass data object 1202. This includes diagnostic findings data objects 1205 of various types including TNM staging data objects, molecular/biomarker data objects, tumor size data objects, and other pathology/image findings data objects, as shown at the top of FIG. 12.

Each of these data objects can store corresponding data elements of configured data types. For example, the tumor size data object is configured to store data elements corresponding to the data types greatest dimension, additional dimension, units, and date. An other pathology/imaging findings data object can be configured to store data elements corresponding to the data types type, value, and date. A finding can be any kind of information about an anatomic site, obtained from one or more samples from the site, which may originate from a report. For example, from a pathology report, findings such as a histologic grade can be extracted; from an imaging report, findings such as the tumor size can be extracted, and so forth. In the data schema 1200, findings are generally tied to a particular site, although some findings may be related directly to the cancer condition itself and not a particular site. For example, cancer stage may be defined at a higher level rather than an individual site. In the patient summary UI as shown in FIGS. 9B-9E, these diagnosis data objects correspond to the data category events 910 displayed in the top row, one instance of which is the cancer diagnosis event 922.

Another data object in the diagnosis 1203 category is a tumor mass(es) data object 12002. The tumor mass data object stores data elements characterizing tumors, organized according to the data types histology, anatomic site, site description, and behavior. For example, the tumor mass data object 1202 includes a structured field for the data type “behavior,” which indicates whether the tumor is a primary tumor, metastatic tumor, or benign.

There may be separate data objects, connected to the root data object 1201, for multiple tumor masses. There can be multiple instances of the tumor mass object, each corresponding to a different tumor mass identified at a different location in the patient. A tumor mass can be designated as a primary cancer or a metastasis, which will affect the network interconnections to other objects. Thus, the data objects can include a data object corresponding to a primary cancer, another data object corresponding to a metastasis of that primary cancer, etc. As shown in FIG. 12, for each tumor mass, the data object can include information such as histology, anatomic site, site description, and behavior. This data object is linked to various other diagnosis-related data objects 1203, including cancer conditions, diagnostic findings 1205, and reports 1204.

One or more treatment-related data objects correspond to treatment, and are connected to the patient root data object 1201 and/or the tumor mass data object 1202. Treatment-related data objects include oncology treatment(s) data object 1208. Oncology treatment(s) data object 1208 is configured to store data elements of type treatment type, date(s), response, and can be linked to an associated report. Oncology treatment(s) data object 1208 can be used to populate the treatments 916 row of the patient journey interfaces of FIGS. 9B-9E.

One or more reports data objects 1204 can be connected to the patient root data object 1201 and/or the diagnostic finding(s) data object 1205. Reports from an EMR or other source can also be stored as a report(s) data object 1204. As shown in FIG. 12, report(s) data object 1204 is configured to store data types status, category, title, date(s), attachment(s). Report(s) data object 1204 can include attachments or addendums in the form of a PDF or image. Addenda are issued when changes are made to a patient's clinical documentation and medical records. They may include information that was not available at the original time of entry, or include corrections to previously issued medical information. It is important for the clinician to know if a particular patient report was updated or added to, and also view a report in its entirety. Report(s) data object 1204 can also include text data extracted from the reports (e.g., using OCR).

One or more history-related data objects 1210 can be stored and connected to the patient root data object 1201 and/or the tumor mass data object 1202. History-related data objects 1210 can include various different types of data objects with corresponding attributes, as shown in FIG. 12. For example, data schema 1200 can include a medication(s) data object, a comorbidities data object, a family medical history data object, a surgical history data object, an allergies data object, a substance abuse data object, a performance status data object, an environmental risks data object, a social history data object, and an other history findings data object, as depicted in FIG. 12. History-related data elements of various data types as shown in FIG. 12 can be stored to the history-related data objects 1210. The data mappings shown in FIG. 12 can be used to establish where in the various interface views the corresponding data elements will be displayed.

In the data schema 1200, each data object can be stored in association with one or more timestamps. The timestamps can track when an event happened. For example, a given data object can include a timestamp corresponding to the day and/or time of a diagnosis, treatment, sample collection, procedure date, report issue, or other event. The timestamps can further track when data was integrated into the unified patient database. For example, when data is stored to the unified patient database, medical data processing system 200 generates and stores a timestamp indicating the time at which the data was incorporated into the unified patient database.

B. Data Schema Example

FIG. 13 shows an example data schema 1300 according to some embodiments. The data schema 1300 includes data objects for different cancer sites. Cancer 1 1302 and cancer 2 1307 are primary cancers. Each of these is stored as its own data object with associated information such as stage, diagnosis, etc., stored to that data object.

At a first time T1, cancer 1 can be associated with multiple data objects in the data schema 1200 of the unified patient database illustrated in FIG. 12, including a tumor mass 1202. Other objects for findings associated with cancer 1 are linked to the tumor mass including TNM staging objects, biomarker objects, tumor size objects, and so forth.

At later times, other sites can be found and associated with the primary cancers, e.g., cancer 1 or cancer 2. As a new site (tumor mass) is identified, the new tumor mass object can be linked in the data model. For example, as shown in FIG. 13, a mass 1 data object 1306 is stored in association with the primary cancer 1 data object 1302. The mass 2 data object 1308 and mass 3 data object 1310 can be two data objects are stored in association with the cancer 2 data object 1304. Mass 1 data object 1306, mass 2 data object 1308, and mass 3 data object 1310 can correspond to multiple tumor mass objects 1202 linked to a same patient object 1201, as shown in FIG. 12.

The example depicted in FIG. 13 illustrates how the data schema of FIG. 12 can be used to handle a diagnostic journey that a cancer patient might go through, which may include various testing, imaging, and other diagnostics, with new information coming in over time. This data schema is set up to be able to be updated while maintaining complex relationships of different data types from different data sources coming in at different times.

As shown on the right hand side, each of the objects for the masses 1306, 1308, and 1310 have associated data elements for storing information such as site, size, liver, site, and, if the data came from a report, that report is also stored as a data element to that data object (e.g. reports 1312, 1214, 1316, and 1318 and associated data attributes that may be extracted from these reports). For example, using the interfaces shown in and described above with respect to FIGS. 3A-7B, information is extracted from the report. The data elements can be assigned a data category using NLP which is then used to populate the appropriate data object.

Each of the data objects 1306, 1308, and 1310 can correspond to three hypothetical time points. Each time point represents a time at which the data populating the corresponding data object was obtained. For example, data object 1306 is populated with data which originates from a radiology report PDF 1312 that was obtained on a given date, data object 1308 is populated with data which originates from a pathology report 1314 which was obtained at a later date, and data object 1310 is populated with data which originates from another pathology report 1316 obtained at a given date. Each of these can be ingested into the unified patient database at different respective times, tracked with timestamps stored to the respective data objects.

For example, at a first time point, based on a radiology report 1312, a lung mass is discovered in the patient's lung. At this time point, other tests are pending. The data schema 1300 can be updated as additional information becomes available. The initial data objects may correspond to initial assumptions about the patient's diagnosis. For example, there is two-centimeter mass in the lung, and another centimeter mass in the liver. There is a primary diagnosis entered that there is a primary lung cancer that has probably metastasized to the liver. The doctor may they send for additional tests. In this example, the report 1312 is connected to two masses 1306 and 1308, indicating that, at the time report 1312 was obtained, both masses 1306 and 1308 were included in the radiology analysis and corresponding data was extracted.

At Time Two, when additional test results 1314 and 1316 come back, two more reports are added to the data schema 1300. Report 1314 is a pathology report pertaining to mass 1 1306. At time two, the pathology report 1314 is ingested into the system and NLP is used to identify data categories corresponding to data fields extracted from the radiology report 1314 and populate corresponding data objects including a tumor mass data object 1202 corresponding to mass 1 1306 and linked data objects corresponding to related findings. Report 1316 is a pathology report pertaining to mass 1 1306. At time two, the pathology report 1316 is also ingested into the system and NLP is used to identify data categories corresponding to data fields extracted from the pathology report 1316 and populate corresponding data objects including a tumor mass data object 1202 corresponding to mass 2 1308 and linked data objects corresponding to related findings.

At Time Three, once a colonoscopy report 1318 is retrieved by medical data processing system 200, additional colonoscopy findings are abstracted from that report. This helps the user to make additional diagnoses such as to confirm that the liver mass matches the colon mass that was found from the colonoscopy. A final picture of the patient's diagnosis can then be created. In this example, the diagnosis includes a lung cancer as well as a colon cancer that has metastasized, with two different primary diseases at the same time.

The data schema depicted in FIGS. 12 and 13 facilitates representation of all of these three states as snapshots in time but also allows a user to change the relationships between entities as new information from new reports becomes available. The data schema provides for representations of the reports themselves, as well as representations of the individual findings that were abstracted from the reports. The data schema also provides a representation of each cancer and anatomic site, and attributes of these sites. Each data object is associated with one or more timestamps, so the journey of a patient can be tracked over time to better facilitate the clinical decision making process. The data schema links sites, finding and reports, while allowing the site to be related to the latest piece of information. Some of these relationships can be modified individually without impacting the rest of the graph of data elements and attributes. When these associations are created, there is a timestamp associated with the association. Thus, the data schema facilitates the interface views that provide a visibility into not only when a report was created, but new associations, old associations, and the changes in associations over time as well. The data schema can also track provenance information (e.g., who edited something and where).

### V. Methods

A. Medical Data Workflow Overview

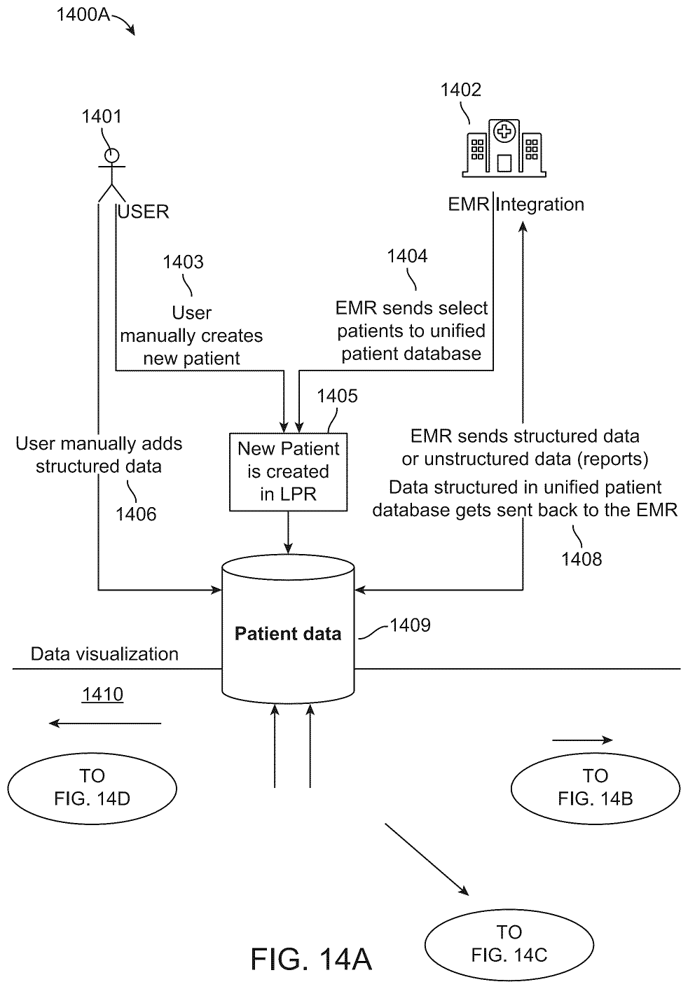

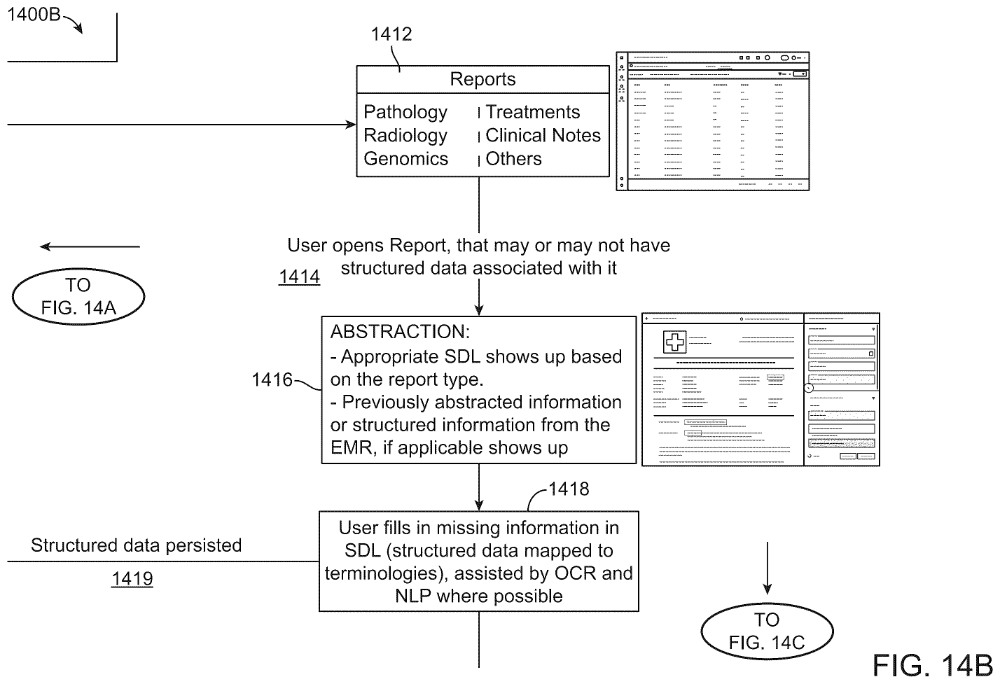

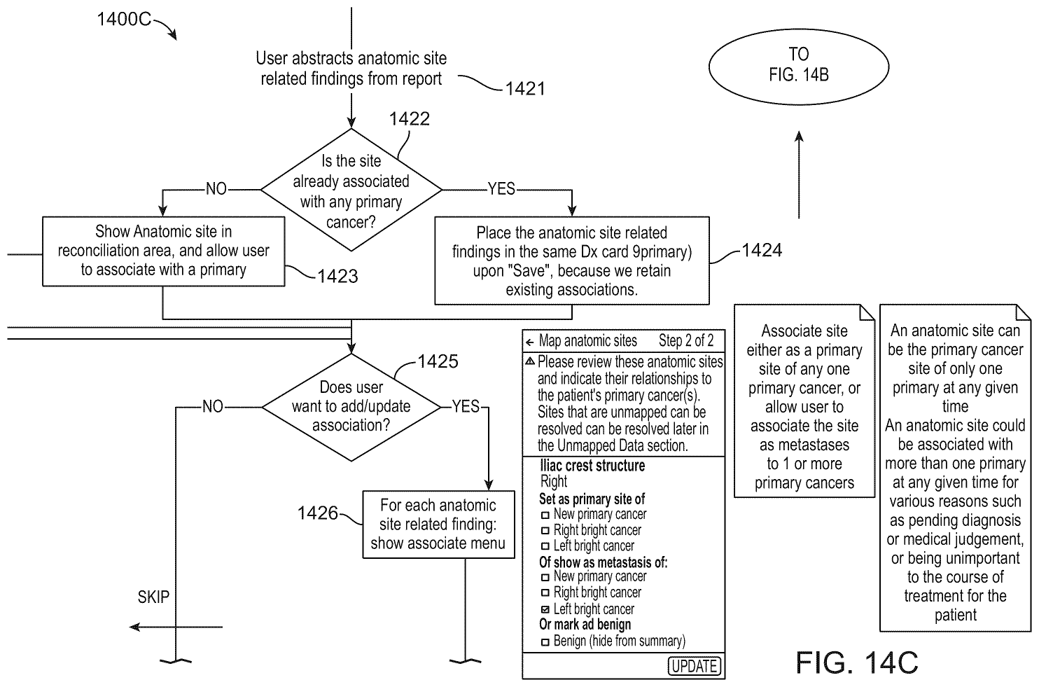

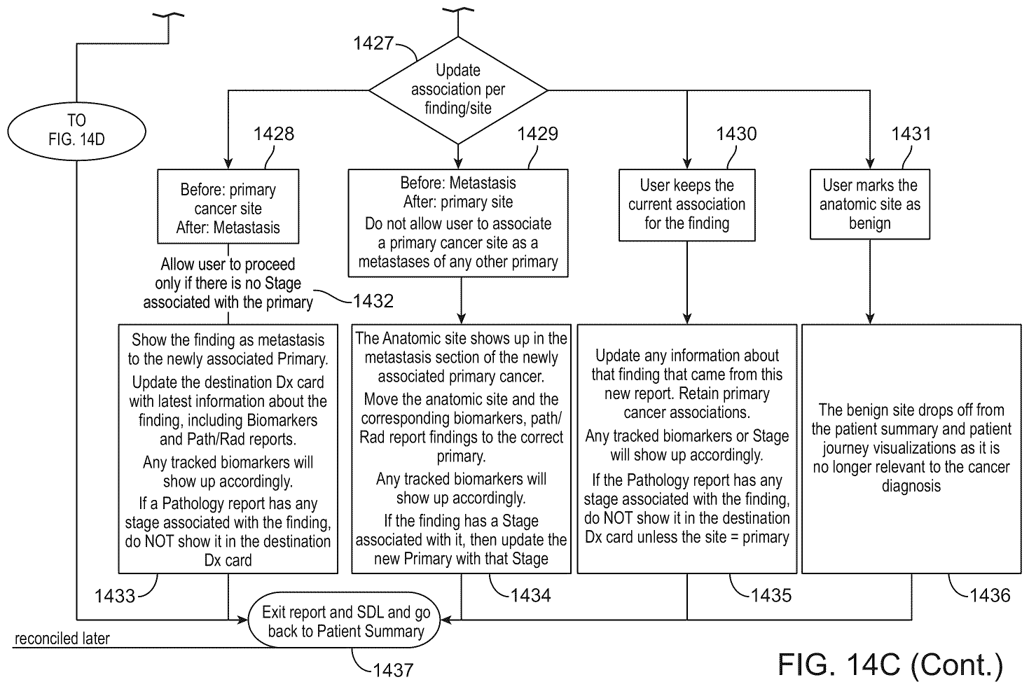

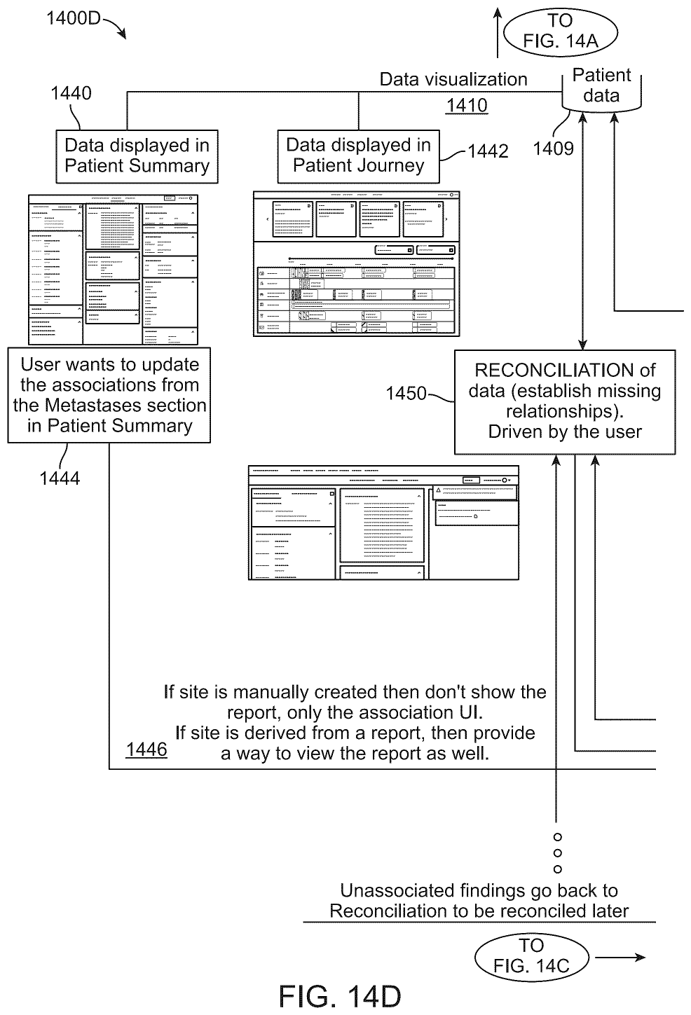

FIGS. 14A-14D illustrate an overview of an oncology workflow for ingestion, modification, and display of patient data. The workflow of FIGS. 14A-14D includes gathering and storing data to a unified patient database 1409 (e.g., the unified patient database 204 depicted in FIG. 2). This data can include relevant radiographic, procedural, and pathologic findings related to one or more primary tumors and their associated metastatic lesions, which can be updated through the course of cancer treatment and other facets of the patient journey. The data can also be retrieved from the unified patient database 204 and displayed in a series of interface views that facilitate clinical patient management, care, and diagnostics. FIGS. 14A-14D provide an overview of operations which are described in further detail with respect to the methods of FIGS. 15-21.

In FIG. 14A, data is gathered and stored to the unified patient database 1409. First, a patient record is created, which can originate via input from a user 1401 and/or EMR integration 1401. A user can manually create a new patient at 1403. The EMR can send select patients to the system at 1404. This data can come from an EMR, or other external databases such as could be lab systems, or other data systems in a hospital. This can result in patient data such as a patient identifier and other data types which may be stored to a patient root data object 1201 as shown in FIG. 12. Data gathered at 1403 and 1404 includes patient data identifying a patient. If there is not a preexisting record for the patient, a new record is created.

Additional data can then be stored to the unified patient database, e.g., as additional data is gathered and/or periodically. At 1408, the EMR sends reports to the system. The system generates structured data from the reports and sends the structured data to the unified patient database 1409 for storage in association with the patient record. This can be performed using the interfaces shown in and described above with respect to FIGS. 4A-7B. At 1406, the user manually adds structured data, which is stored to the unified patient database 1409. This can be performed using the interfaces shown in and described above with respect to FIGS. 3A-3H. The data can be stored according to the data schema described above with respect to FIGS. 12 and 13. Thus, the system can gather both structured and unstructured data from disparate sources and store it in a unified fashion in the unified patient database 1409.

The data stored to the unified patient database 1409 can include identifying information about the patients and the patient demographics. The data stored to the unified patient database can include structured data about the patient's diagnosis, medications, medical history, etc. The data stored to the unified patient database can also include unstructured data such as pathology reports, imaging reports, clinical notes, and so forth. For example, as shown in FIG. 12, data can be stored to data objects that are mapped to one another and can be updated and modified over time. As shown in FIG. 13, specific instances of these data objects may store the reports themselves in association with data which has been extracted from these reports.

If all the data stored to the unified patient database is in a structured form, that data can be used to generate various analytics or visualizations as described above with respect to Section III, e.g., the patient summary and the patient journey views. Before this can be achieved, data enrichment operations are performed on the data that comes from the EMR or other external database/system.

In FIG. 14B, data abstraction of reports 1412 is performed. The reports 1412 can include pathology reports, treatments, etc., as shown in FIG. 14B. At 1414, a user opens a report. The report may or may not include structured data. The user may open a report for display. Based on which report is opened, the list of the fields that can be populated using information that resides in this report may vary. For example, as shown in FIG. 6A, the interface 600 for data abstraction shows a surgical pathology report and a corresponding set of fields to be populated which are associated with surgical pathology reports. As shown in FIG. 6B, the interface 625 for data abstraction shows a radiology report and a corresponding set of fields to be populated which are associated with radiology reports.

At 1416, abstraction is performed. Certain fields or medical concepts may be highlighted in the data abstraction UI for the user to provide information such as diagnoses, notes, etc. At 1418, the user fills in missing information. The structured data is mapped to terminologies, assisted by OCR and NLP where possible. This process generates structured data from the unstructured report, and the structured data is persisted at 1419. Once the user saves all of this information, it is immediately sent to the unified patient database. This data is enriched by adding more structured information that has been taken out from this report, and sending it back to the unified patient database.

In FIG. 14C, further detail is shown as to the data abstraction process. At 1421, a user abstracts anatomic site related findings from a report. At 1422 the system determines whether the site is already associated with any primary cancer. This may be achieved, for example, via user input to the interface providing or confirming an association. If the site is already associated with a primary cancer, at 1424, the system stores the anatomic site findings in association with that primary cancer, upon save, to retain existing associations. If the site is not already associated with a primary cancer, at 1423, the system shows the anatomic site in the reconciliation area, and allows the user to associate the anatomic site with a primary. Then, the flow proceeds to the reconciliation process described below with respect to FIG. 14D.

At 1425, the system determines whether the user wants to add/update an association. If the user does not want to add or update an association, then the add/update process is skipped. If the user does want to add or update an association, then at 1426, for each anatomic site related finding, the system shows an associate menu. Via the associate menu, the user can associate a site either as a primary site of any one primary cancer, or allow the user to associate the site as metastases to one or more primary cancers. In some implementations, an anatomic site can be the primary cancer site of only one primary at a given time. An anatomic site could be associated with more than one primary at any given time for various reasons, such as pending diagnosis or medical judgment, or being unimportant to the course of treatment for the patient.

There are several options for these association updates. At 1428, before, the site is labeled a primary cancer site and after, the site is labeled a metastasis. Then at 1432, the user is allowed to proceed only if there is no stage associated with the primary. At 1433, the system shows the finding as metastasis to the newly associated primary and updates the data object with the latest information about the finding, including biomarkers and pathology/radiology reports. Any tracked biomarkers will show up accordingly. The system also allows the user to choose and track which of the many biomarkers are critical to the description of the cancer. Biomarker information can be presented up front in the patient summary view. In addition to the patient summary interface views depicted in FIGS. 8A-8C, the patient summary interface may further include interface views that display relevant biomarkers and accept user input to add, modify, or drill down to view more detailed biomarker data.

At 1429, the site is labeled, as described at 1427, the association is updated per finding/site. For example, before, a site is designated as a metastasis. This is updated such that the site is associated with a primary site. At 1434, the anatomic site shows up in the metastasis section of the newly associated primary cancer. The system moves the anatomic site and the corresponding biomarkers and pathology/radiology report findings to the correct primary. Information such as biomarkers, findings, etc. can be stored in connection with a different primary cancer object, using the connections of data objects described above with respect to FIGS. 12 and 13. Any biomarkers will show up accordingly in association with the updated primary cancer object. For example, if the finding has a stage associated with it, then the new primary is updated with that stage.

At 1430, the user keeps the current association for the finding. In the case of keeping the current association, at 1435, the system updates any information about that finding that came from this new report. Primary cancer associations are retained. Any tracked biomarkers or stage will show up accordingly. If the pathology report has any stage associated with the finding, the stage information may not be shown in the patient summary unless the site is a primary site.

At 1431, the user marks the anatomic site as benign. If the user marks the anatomic site as benign, at 1436, the benign site drops off from the patient summary and patient journey visualizations as it is no longer relevant to the cancer diagnosis. At 1437, the report is exited and the interface transitions back to the patient summary view.

FIG. 14D illustrates a data visualization 1410 portion of the work flow. This can include retrieving data associated with a patient from the unified patient database and displaying a user interface such as a patient journey interface or patient summary interface.

At 1442, data is retrieved from the unified patient database 1409 and displayed in the patient journey. Based on an identifier of a patient, a patient record in the unified patient database is identified. This can include a patient root data object 1201 which can be identified by querying the unified patient database to identify the patient object corresponding to that identifier. As shown in FIG. 12, the patient root data object 1201 is mapped to various different data objects which can be timestamped and used to visualize the patient journey over time. Examples of patient journey interfaces are shown in FIGS. 9A-9E.

At 1440, data is retrieved from the unified patient database 1409 and displayed in the patient summary. Based on an identifier of a patient, a patient record in the unified patient database is identified. This can include a patient root data object 1201 which can be identified by querying the unified patient database to identify the patient object corresponding to that identifier. As shown in FIG. 12, the patient root data object 1201 is mapped to various different data objects which can be timestamped and used to populate the patient summary interface. Examples of patient summary interfaces are shown in FIGS. 8A-8C. From the patient summary, the user can perform updates, e.g., if the user wants to update the associations from the metastases section in patient summary at 1444. This can trigger display of a modified UI at 1446. If a site is manually created, no report is shown, only the association UI. If the site is derived from a report, then the report is also viewable.

At 1450, reconciliation of data is performed. The user can interact with the GUI to establish missing relationships (e.g., to associate an identified cancer mass with a particular primary site, etc.). Unassociated findings are flagged to be reconciled later. In reconciliation, unmapped data is identified. In one example, a cancer mass does not specify an associated primary site. In another example, a surgical history record does not specify whether is it an oncology surgical history or a non-oncology surgical history. As another example, reconciliation can be used to identify a stage of a cancer. Reconciliation can be used both to identify missing relationships and fill in those missing relationships, as well as determine where in the UI it is appropriate to display a particular piece of information. Reconciliation can be guided using the interfaces depicted in FIGS. 7A-7B.

B. Data Management Techniques

FIG. 15 illustrates a method 1500 of managing patient data from disparate sources in an integrated fashion. Method 1500 can be performed by, for example, medical data processing system 200 of FIG. 2. Method 1500 can be used to integrate both structured and unstructured data from a variety of sources into a unified patient database in a unified and organized fashion so that the data can be used to generate useful visualizations as described herein.

In step 1502, medical data processing system 200 creates a patient record for a patient in a unified patient database. The patient record includes an identifier of the patient and one or more data objects related to medical data associated with the patient. The identifier of the patient may, for example, be the patient's name, an alphanumeric identifier of the patient, or the like. As described above with respect to FIG. 12, the unified patient database can store multiple data objects of different types that organize different types of medical data associated with the patient. For example, the patient record can include a data object corresponding to a tumor mass, a data object corresponding to treatments given to the patient, and so forth.

The unified patient database includes data from a plurality of sources (e.g., the data can be ingested to the unified patient database from an EMR, RIS, user entry, wearable devices, etc.). As described above with respect to FIG. 14A, the patient record can be created via user input or from information retrieved from an external database such as an EMR. Creating the patient record can include generating and storing a data object, table, or other record for that patient. The data stored can include information such as the patient identifier, demographic information, date of birth, etc.

In step 1504, medical data processing system 200 retrieves, from an external database, a medical record for a patient. The medical record can include unstructured data such as reports in PDF or image format. Alternatively, or additionally, the medical record can include structured data such as a table. The medical record can be retrieved from one or more external databases including, for example, an EMR (electronic medical record) system, a PACS (picture archiving and communication system), a Digital Pathology (DP) system, a LIS (laboratory information system) including genomic data, RIS (radiology information system), patient reported outcomes, wearable and/or digital technologies, social media etc. The medical record can include information such as a name identifying a particular cancer mass, a timestamp associated with the report, and other information, as described herein. In some implementations, medical data processing system 200 retrieves the medical record based upon the identifier of the patient. For example, medical data processing system 200 queries the unified patient database to identify a record including or indexed by the identifier of the patient. Alternatively, or additionally, medical data processing system 200 may retrieve medical records periodically (e.g., by downloading data from an external database in batches).

The medical record may include structured data and/or unstructured data. For example, the medical record for the patient is structured (e.g., is in a first format). The structured data can include a set of data elements correlated to corresponding data types. Data elements can include a word or group of words corresponding to an element in the medical record, examples of which can include “right breast tumor,” “MRI of Jan. 5, 2021,” and so forth. Each data element can be labeled and/or stored in association with a corresponding data type characterizing the data element, such as “primary tumor,” “treatment,” and so forth. Alternatively, or additionally, the medical record for the patient is unstructured (e.g., in a second format). The unstructured data may include data elements without specifying the data types.

In some embodiments, the medical record includes unstructured data. Medical data processing system 200 may identify text from unstructured data such as a PDF or image. Medical data processing system 200 may apply a first machine learning model to identify text in the medical record. For example, the first machine learning model is or includes an Optical Character Recognition (OCR) model and the text is identified using OCR.

Medical data processing system 200 may apply a second machine learning model to correlate a portion of the identified text with a corresponding field. Medical data processing system 200 may use the second machine learning model to identify a data element such as a word or set of words, and analyze the unstructured data to assign the data element to a data type. For example, upon identifying the data element “colon cancer,” surrounding words and the phrase itself are analyzed to assign the data type “diagnosis” to the data element.

In some aspects, the second machine learning model is or includes a Natural Language Processing (NLP) model. A trained NLP model identifies data types for text in the unstructured report (e.g., the NLP model determines that the text “Jan. 10, 2020” corresponds to a “date” data type/field and the text “radiation” corresponds to a “treatment type” data type/field). Medical data processing system 200 may, for example, use NLP to recognize entities from the input text strings. A NLP model may identify entities corresponding to pre-defined medical categories and classifications, such as medical diagnoses, procedures, medications, specific locations/organs in the patient's body, etc. This can be performed in some implementations using a named entity recognizer trained on medical data to recognize entities corresponding to the data types of interest. Each entity can be labeled with a data type that indicates the category/classification, and specifies a data element or value corresponding to the data being categorized. Medical data processing system 200 can then generate structured medical data that associates the data types with the data elements based on the mapping. Techniques for processing unstructured medical data using machine learning are described in further detail in PCT Publication WO 2021/046536, supra.

In some implementations, medical data processing system 200 is communicatively coupled to multiple external databases/systems, including an EMR, PACS, DP, etc. When these systems make changes to data associated with one or more patients managed by the medical data processing system 200, the data is transmitted to the medical data processing system 200. The medical data processing system 200 can periodically pull medical records from the one or more external databases to periodically update the unified patient database.

In step 1506, medical data processing system 200 receives identification of a primary cancer associated with the medical record via a Graphical User Interface (GUI). For example, an abstraction process can be performed using an association interface such as that shown in FIG. 7B. The user can associate the cancer mass identified in the report with a particular primary cancer site. In some embodiments, receiving the identification of the primary cancer associated with the medical record includes displaying, via the GUI, the medical record and a menu configured to receive user input selecting one or more primary cancers and receiving, via the graphical user interface, user input selecting the primary cancer.

In some cases, such user selection is performed in the course of a reconciliation process, as described above with respect to FIGS. 7A and 7B. For example, the medical record is stored in the patient record. Medical data processing system 200 parses the medical record to determine that the patient record is not associated with a particular primary cancer. Medical data processing system 200 displays the medical record and the menu responsive to determining that the patient record is not associated with a particular primary cancer, prompting the user to reconcile the data via an interface such as that depicted in FIGS. 7A and 7B.

Alternatively, or additionally, medical data processing system 200 receives identification of a potential primary cancer associated with the medical record from the external database (e.g., an EMR). Such an identification received from a remote database may be confirmed via user input to the GUI in some cases. For example, medical data processing system 200 identifies the primary cancer by analyzing the data elements and the data types. A particular data element (e.g., “left breast cancer”) may, for example, be labeled with a data type indicating that the data element corresponds to a primary cancer (e.g., “primary cancer”). In some implementations, data abstraction module 232 extracts medical data from a document file and maps the extracted data to a particular primary cancer. The mapping can be based on a master structured data list (SDL) that defines a list of data categories for a document type of the document.

Medical data processing system 200 may display the GUI with a prompt for a user to confirm the primary cancer identification (e.g., with a prefilled field, which may be highlighted and/or flagged with text prompting the user to confirm or modify the primary cancer designation). Medical data processing system 200 may then receive user confirmation of the primary cancer identification via the GUI. Alternatively, or additionally, medical data processing system 200 can identify a primary cancer without user intervention in some cases. For example, the data element may be stored to the unified patient database in association labeled with a data type (e.g., a structured field) that indicates the “behavior” of the tumor, as shown in FIG. 12, which may whether the tumor is a primary tumor, metastatic tumor, or benign.

In some cases, identifying the primary cancer can include analysis of unstructured data by medical data processing system 200. For example, a medical record is received in an unstructured format including unstructured data. Medical data processing system 200 identifies, from the unstructured data, a data element associated with the primary cancer and analyzes the unstructured data to assign the data element to a data type. This may be performed using one or more machine learning models as described above with respect to step 1504.

In step 1508, medical data processing system 200 stores the medical record linked to a primary cancer object in the patient record in the unified patient database. Storing the medical record may include storing identified text to the unified patient database in association with an identified field, using the data schema described above with respect to FIGS. 12 and 13.

In step 1510, medical data processing system 200 receives, via user input to the GUI, medical data for the patient. This may be medical data directly entered using the interfaces shown and described above with respect to FIGS. 3A-3H. For example, a user may enter treatment information, diagnosis information, information about a metastasis of the primary cancer, and so forth, into corresponding fields of the GUI.

In step 1512, medical data processing system 200 determines that the medical data for the patient is associated with the primary cancer. For example, data entered into the GUI by the user may be entered into a field designated for the primary cancer. As another example, data retrieved from the external database indicates that the medical data for the patient is associated with the primary cancer. The medical data processing system 200 may compare the field received at 1510 to a corresponding stored data element in the unified patient database corresponding to the medical record retrieved at 1504.

In step 1514, medical data processing system 200 stores the medical data for the patient linked to the primary cancer object in the patient record in the unified patient database. The data elements can be linked using the data schema described above with respect to FIGS. 12 and 13.

The data stored to the unified patient database can be efficiently retrieved and displayed for a user. For example, medical data processing system 200 retrieves, from the unified patient database, at least a subset of the medical data for the patient. Medical data processing system 200 causes display, via a user interface, of the at least the subset of the medical data for the patient for performing clinical decision making. Causing display may include displaying the user interface on a display component of medical data processing system 200 itself, or transmitting instructions useable by an external computing device to display the user interface. The displayed information is displayed in a user-friendly manner to facilitate clinical decision making, via interfaces such as those depicted in FIGS. 7A-10.

C. Techniques for Data Management

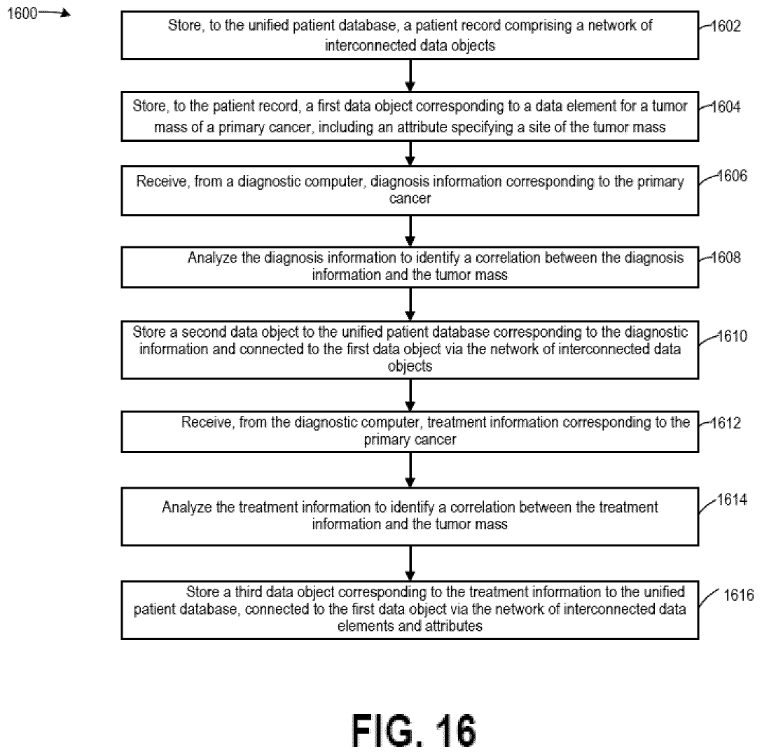

FIG. 16 illustrates a method 1600 of managing a unified patient database using a data schema such as that depicted in FIG. 12. The data schema can be used to manage patient data to facilitate efficient generation of the interface views depicted herein for ease of clinical decision making, as well as facilitate exportation of structured medical data. Method 1600 can be performed by, for example, medical data processing system 200 of FIG. 2.

In step 1602, medical data processing system 200 stores, to the unified patient database, a patient record comprising a network of interconnected data objects. As described above, the unified patient database can include data from multiple sources, such as data integrated from an EMR system, provided via user input to an interface on a remote computer, gathered from wearable device, and so forth.

In step 1604, medical data processing system 200 stores, to the patient record in the unified patient database, a first data object corresponding to a data element for a tumor mass of a primary cancer, the first data object including an attribute specifying a site of the tumor mass. In some implementations, initial data is uploaded to the unified patient database from one or more of the multiple sources. As a given data element (e.g., information corresponding to a particular field, such as information characterizing a tumor mass) is ingested into the system, the medical data processing system 200 creates a data object to which to store this information. The data object may be created responsive to data being obtained from disparate sources. For example, a user may enter data from the user interface. Some data may be automatically ingested from an external system such as an EMR. Additional structured data may be automatically abstracted from documents (e.g., PDFs) and verified by the user. The data object can further include one or more data attributes, including one that specifies the site of the tumor mass (e.g., right lung, left breast, and so forth).

In step 1606, medical data processing system 200 receives, from a diagnostic computer, diagnosis information corresponding to the primary cancer. Medical data processing system 200 may, for example, receive, over a network, information that a doctor has input into a user interface provided by medical data processing system 200. As a specific example, using a GUI such as that depicted in FIGS. 4B and 4C, a doctor can input diagnostic information such as findings and biomarkers. Such information can be gathered in a structured fashion based on the input data fields of the GUI.

In step 1608, medical data processing system 200 analyzes the diagnosis information to identify a correlation between the diagnosis information and to the tumor mass. This may involve, for example, traversing the data received from a GUI. As a specific example, as depicted in FIG. 4C, the GUI includes fields for an tumor site information 420a as well as biomarker information 420b. When medical data processing system 200 receives data from such a GUI it can determine that the tumor site (e.g., primary tumor mass) is associated with the biomarkers. Alternatively, or additionally, the diagnosis information can come from an unstructured report, and medical data processing system 200 can apply one or more machine learning models to identify data types and correlations, as described above with respect to step 1504 of FIG. 15.

In step 1610, based on identifying the correlation between the diagnosis information and the tumor mass, medical data processing system 200 stores, to the unified patient database, a second data object corresponding to the diagnostic information, the second data object connected to the first data object via the network of interconnected data objects. The second data object may include one or more attributes such as a stage of the primary cancer, a biomarker, and/or a tumor size. Medical data processing system 200 can store the data object connected to the first data object using the data schema described above with respect to FIGS. 12 and 13.

In step 1612, medical data processing system 200 receives, from the diagnostic computer, treatment information corresponding to the primary cancer. The treatment information may be received from the diagnostic computer in a similar fashion as the diagnosis information, as described above at step 1606. For example, the treatment information can be retrieved from the diagnostic computer via input to a GUI, analysis of an unstructured report, or other suitable means.

In step 1614, medical data processing system 200 analyzes the treatment information to identify a correlation between the treatment information and to the tumor mass. Medical data processing system 200 may, for example, analyze structured fields and/or perform NLP on text data, in a similar fashion as described above with respect to step 1608.

In step 1616, based on identifying the correlation between the treatment information and the tumor mass, medical data processing system 200 stores, to the unified patient database, a third data object corresponding to the treatment information, the third data object connected to the first data object via the network of interconnected data objects.

Medical data processing system 200 may also receive and store patient history data such as surgical history, comorbidities, medications, and other family history, as described above with respect to FIG. 12. For example, medical data processing system 200 receives patient history data. The patient history data may be received from the diagnostic computer (e.g., via direct user input). Alternatively, or additionally, the patient history data may be received from an external computing system such as an EMR. Medical data processing system 200 analyzes the patient history data to identify a correlation between the patient history data and the tumor mass (e.g., in a similar fashion as described above with respect to step 1608). Based on identifying the correlation between the patient history data and the tumor mass, medical data processing system 200 stores, to the unified patient database, a fourth data object corresponding to the patient history data. The fourth data object is connected to the first data object via the network of interconnected data objects.

Medical data processing system 200 may also receive and store information about additional tumor masses such as a tumor mass at a metastasis site of the primary cancer, a tumor mass associated with another primary cancer, and so forth. For example, medical data processing system 200 receives, from the diagnostic computer, tumor mass information corresponding to a tumor mass at a metastasis site of the primary cancer. A user may enter the tumor mass information into a GUI or upload a document, and data can be transmitted to medical data processor via the GUI in a similar fashion as described above with respect to step 1606. Medical data processing system 200 analyzes the tumor mass information to identify a correlation between the diagnosis information and the tumor mass (e.g., in a similar fashion as described above with respect to step 1608). Based on receiving the tumor mass information and identifying the first data object, medical data processing system 200 stores, to the unified patient database, a fifth data object corresponding to the tumor mass information connected to the first data object via the network of interconnected data objects.

Medical data processing system 200 may subsequently update the unified patient database. For example, medical data processing system 200 imports medical data from an external database. The external database may correspond, for example, to one or more of an EMR (electronic medical record) system, a PACS (picture archiving and communication system), a Digital Pathology (DP) system, an LIS (laboratory information system), and/or a RIS (radiology information system). In some examples, medical data processing system 200 parses the imported data to identify a particular data element associated with the patient and the primary cancer. Medical data processing system 200 can, for example, parse structured data received from an EMR or other source to identify a field noting that the data element describes a treatment, tumor mass, or other type of medical data. The structured data may further note the primary cancer corresponding to the first data object (e.g., a field in ingested structured data may indicate that a treatment was applied for the primary cancer). Medical data processing system 200 may then store the particular data element in association with the first data object (e.g., to a sixth data object). For example, the sixth data object is linked to the first data object in a data schema similar to that depicted in FIG. 12.

As described above with respect to FIG. 12, the data stored to the unified patient database can be indexed using timestamps. The timestamps can track when an event happened (e.g., the day and/or time that a MRI was taken or a treatment was administered or a diagnosis was given). The timestamps can further track when data was integrated into the unified patient database. For example, upon generating each of the first data object and the second data object, medical data processing system 200 generates a first timestamp stored in association with the first data object indicating the time of creation of the first data object. Medical data processing system 200 generates a second timestamp stored in association with the second data object indicating the time of creation of the second data object. These timestamps are then stored to the respective data object, and can be used to show history of the database entries. The timestamps tracking when each event happened can further be used to generate chronological visualizations such as the patient journey views shown in FIGS. 9A-9E.

The data stored to the unified patient database can be efficiently retrieved and displayed for a user. For example, medical data processing system 200 retrieves, from the unified patient database, one or more of the attributes specifying the site of the tumor mass, the diagnosis information, and/or the treatment information. Retrieving the attributes may include querying the unified patient database. In some aspects, medical data processing system 200 traverses the connections between the data objects to identify associated data objects. For example, medical data processing system 200 may identify a pointer from a data object corresponding to a tumor mass to another data object corresponding to treatment of the tumor mass, and retrieve the treatment information therefrom.

Medical data processing system 200 may cause display, via a user interface (e.g., a GUI such as those depicted in FIGS. 8A-9E) of one or more of the attribute specifying the site of the tumor mass, the diagnosis information, and/or the treatment information for clinical decision making. For example, referring to FIG. 8B, the patient summary interface 800 shows an attribute specifying the site of the tumor mass, “right breast 2:00 position,” at 802. The GUI 800 also displays diagnostic information such as the stage and “invasive ductal carcinoma” on the left hand side. The patient summary interface 800 also shows treatment information such as the Oncologic treatments at 806. Similarly, in the patient journey views 9A-9E, information including tumor mass site information, diagnostic information, treatment information, and other information, can be shown in a timeline view. Causing display may include displaying the GUI on a display component of medical data processing system 200 itself, or transmitting instructions useable by an external computing device to display the GUI. The displayed information is displayed in a user-friendly manner to facilitate clinical decision making, as a medical professional can view the information all in one place in an organized fashion that shows the patient's responses over time.

The data stored to the unified patient database can also be efficiently provided to an external system such as an EMR in structured form. For example, medical data processing system 200 identifies, from the unified patient database, a data element and a data type associated with the patient. Medical data processing system 200 transmits, to an external system, the data element and the data type in structured form. As noted above, some data objects or data fields may be populated by integration, but these data can often be unstructured or semi-structured. Using the techniques described herein, a user and/or machine learning can add more details or relationships between the data objects (e.g., via reconciliation or the abstraction tool). This facilitates storing the data to the unified patient database with structured information (e.g., characterizing different data elements as different data types). Such structured data can then be leveraged to send the structured data to an external system such as an EMR if needed.

D. Techniques for Displaying Patient Data Via Patient Journey Interface

FIG. 17 illustrates a method 1700 of displaying patient data for ease of navigation and presentation, via a patient journey interface view such as those depicted in FIGS. 9A-9E. The patient journey view can provide a view of how a patient has responded to treatments over time, with different types of data organized by rows, which helps a clinician to better understand and manage the patient's treatment. Method 1700 can be performed by, for example, medical data processing system 200 of FIG. 2.

In step 1702, medical data processing system 200 receives, via a graphical user interface, data identifying a patient. For example, the user may type in a patient name or identifier to the portal, or select a patient identifier from a displayed menu.

In step 1704, medical data processing system 200 receives user input selecting a mode, of a set of selectable modes of the graphical user interface. For example, as illustrated in FIGS. 8A-10, various modes or interface views are available including a patient journey mode, a summary view, and a reports view. For example, the user selects the patient journey view. Medical data processing system 200 detects a user clicking on the patient journey tab shown near the top of the interface 800 depicted in FIG. 8B, causing the view to transition to the patient journey view.

In step 1706, based on the identification data and the user input, medical data processing system 200 retrieves a set of medical data associated with the patient from a unified patient database. The set of medical data corresponds to the selected mode. For example, the set of medical data corresponds to the patient journey mode. The medical data processing system 200 may query the unified patient database to identify a record for the patient (e.g., by identifying a patient data record as shown in FIG. 12).

Retrieving the set of medical data may include querying a unified patient database to identify a patient record for the patient from the unified patient database. The patient record can include a patient object such as patient root data object 1201 depicted in FIG. 12. Based on the patient object, objects connected to the patient object are identified. Some or all of these objects can then be retrieved for display. For example, as shown in FIG. 12, the patient root data object 1201 is connected to many different data objects each of which can store various data elements. The patient journey view can be configured to display some of this information, which the system can identify based on preconfigured data object types and/or elements. The list of data object types to be displayed in the patient journey can be stored in a configuration file. Based on the object types in the list, instances of these object types can be retrieved, e.g., as long as they have a timestamp within a specified time window. For example, as shown in FIGS. 9A-9E, some of the data is not in the time window currently displayed, and will not be fetched for display at a given time. Other data, such as benign tumors, may be stored to the unified patient database but not displayed in the patient journey UI.

The data retrieved may include various data objects and elements described herein, e.g., with respect to FIG. 12. For example, the set of medical data can correspond to (e.g., be retrieved from or in association with) a treatment object in a unified patient database, the treatment object storing a treatment type, date, and response. The set of medical data can alternatively or additionally correspond to a diagnostic finding object in the unified patient database, the diagnostic finding object storing biomarker data, staging data, and/or tumor size data. The set of medical data can alternatively or additionally correspond to a history object in the unified patient database, the history object storing surgical histories, allergies, and/or family medical history.

In step 1708, medical data processing system 200 displays, via the graphical user interface, a user-selectable set of objects in a timeline, the objects organized in rows, each row corresponding to a different category of a plurality of categories, the categories comprising pathology, diagnostics, and treatments. This may correspond to the patient journey views shown in FIGS. 9A-9E. The medical data processing system 200 may retrieve this information from the unified patient database and use it to display the patient journey view. For example, based on the object types defined above with respect to FIG. 12, a corresponding row in the patient journey interface is identified for a particular object. Based on a timestamp associated with that object, the object is placed at a particular time on the timeline of the patient journey view in the identified row. As a specific example, a biomarker object is placed at a particular time in the biomarker row. This is repeated across each element of the medical data retrieved at 1606, which can result in a GUI view such as those depicted in FIGS. 9A-9E.

The graphical user interface can further include a ribbon displayed above the timeline, the ribbon displaying a subset of the objects flagged as significant. For example, as shown in FIG. 9B, there is a summary ribbon 902 that highlights key events. The summary ribbon can highlight key events in an easy to view place, and the user can drill down to look closer at the events and the order they occurred using the timeline view below. This provides an improved user experience, and can help facilitate clinical decision making by giving the user key events and temporal views of events.

The graphical user interface can receive user interaction to prompt display of additional information including reports. Reports can be viewed in detail or in simplified form. In some implementations, a user can hover over an object in the timeline (e.g., MRI 924 shown in FIG. 9B), and medical data processing system 200 retrieves and displays the report. Medical data processing system 200 may detecting user interaction with an object of the set of objects, such as the MRI 924 shown in FIG. 9B. Medical data processing system 200 identifies and retrieves a corresponding report from the unified patient database. For example, as shown in FIG. 12 a data record can include reports 1204 linked to different data objects such as a patient root data object 1201 for the patient, one for diagnostic findings 1205, etc. Medical data processor can traverse such connections in the unified patient database to identify a report associated with an object in the patient summary graphical user interface. Medical data processor can then display the report via the graphical user interface. This provides a convenient way to drill down into the different objects displayed in the patient summary view.

From the patient journey view, the user can switch to other available views, such as the patient summary view or reports view. For example, the graphical user interface further includes an element for navigating to a second interface view, such as the selectable reports element 812 and summary element 815 depicted in FIG. 8B. Medical data processing system 200 detects user interaction with the element for navigating to the second interface view. For example, the second interface view is the summary view, as shown in FIGS. 7A and 8A-8B, and the second interface view displays oncologic summary data. As another example, the second interface view is the reports view, displaying a particular report or list of reports, e.g., as depicted in FIG. 10.

E. Techniques for Managing and Displaying Multiple Tumor Mass Data

FIG. 18 illustrates a method 1800 of displaying patient data for ease of navigation and presentation, via a side-by-side tumor mass view such as that depicted in FIG. 8C. Method 1800 can be performed by, for example, medical data processing system 200 of FIG. 2.

In step 1802, medical data processing system 200 stores, to a unified patient database, a patient record. The patient record includes a plurality of data objects including a first primary cancer data object storing data elements corresponding to a first tumor mass of a patient and a second primary cancer data object storing data elements corresponding to a second tumor mass of the patient. For example, one object can be stored in association with a primary cancer in the right breast, and another data object can be stored in association with another primary cancer in the right lung. As shown in FIG. 13, the data schema used by medical data processing system 200 can include multiple objects for multiple primary cancers, each having respective data objects such as cancer 1 1302 and cancer 2 1304.

As described above, the unified patient database includes data from a plurality of sources, which can include an EMR (electronic medical record) system, a PACS (picture archiving and communication system), a Digital Pathology (DP) system, an LIS (laboratory information system), a RIS (radiology information system), patient reported outcomes, a wearable device, a social media website, and so forth.

In step 1804, medical data processing system 200 renders and causes display of a graphical user interface. The graphical user interface includes a patient summary. As shown in FIGS. 8A-8C, the patient summary view can include information summarizing patient data in the patient record in the unified patient database. The patient summary view can be displayed as described above with respect to FIG. 17.

In step 1806, medical data processing system 200 detects user interaction with an element of the graphical user interface. For example, the patient summary view shows information about primary cancers, and an element for displaying more information about one or more primary cancers. As a specific example, the patient summary view shown in FIG. 8B includes a box 802 with information about two primary cancers, “breast cancer” and “lung cancer,” along with an element 805 that the user can interact with to display more information. In some implementations, there is an element configured to initiate showing information about multiple primary cancers. Alternatively, the graphical user interface can display a first element 805 when viewing a first primary cancer (e.g., breast cancer) and a second element 805 when viewing a second primary cancer (e.g., lung cancer). In this case, the user could click each of the two buttons in turn.

In some implementations, medical data processing system 200 identifies a number of primary cancers and displays information about each of the identified primary cancers. For example, medical data processing system 200 stores each tumor mass represented as an independent data object, which has structured data fields indicating behavior of the tumor mass, as shown in FIGS. 12 and 13. This data schema allows medical data processing system 200 to count the number of primary or metastatic tumors when necessary.

In step 1808, responsive to detecting the user interaction, medical data processing system 200 retrieves, from the unified patient database, the data elements from the first primary cancer data object and the second primary cancer data object of the patient record. Medical data processing system 200 may identify one or more primary cancer data objects based on the element interacted with at step 1806, and query the unified patient database to retrieve the data elements associated with the corresponding primary cancer data object(s). In some implementations, each tumor mass is represented as an independent data object, which has structured data fields indicating information about the tumor mass such as its behavior (e.g., primary, metastasis, etc.) as shown in FIG. 12. This allows medical data processing system 200 to identify the primary cancer data objects in the unified patient database.

In step 1810, medical data processing system 200 renders a first modal corresponding to a first primary cancer of a patient and a second modal corresponding to a second primary cancer of the patient. Rendering a modal may include generating graphics to overlay over the current GUI (e.g., as a popup over the patient summary view).

In step 1812, medical data processing system 200 causes display of the first modal and the second modal side-by-side in the graphical user interface. The side-by-side modals can include two pop-up windows overlaid over the patient summary view, as shown in FIG. 8C. As shown in FIG. 8C, the first modal and the second modal can provide a summary of key information about each of the primary cancers. The information displayed in the first modal and the second modal can include a set of biomarkers with timestamps, staging information, and metastatic site information, as shown in FIG. 8C. Showing the primary cancers side-by-side in a summary fashion can help a clinician such as an oncologist to see how multiple primary cancers are progressing at once. Causing display of the modals may include displaying the modals on a display component of medical data processing system 200 itself, or transmitting instructions useable by an external computing device to display the GUI.

F. Diagnostic Workflow Overview

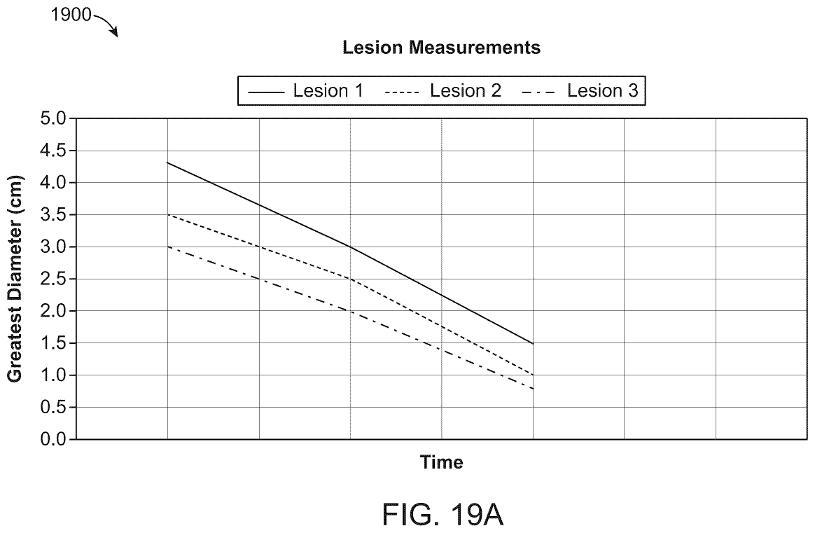

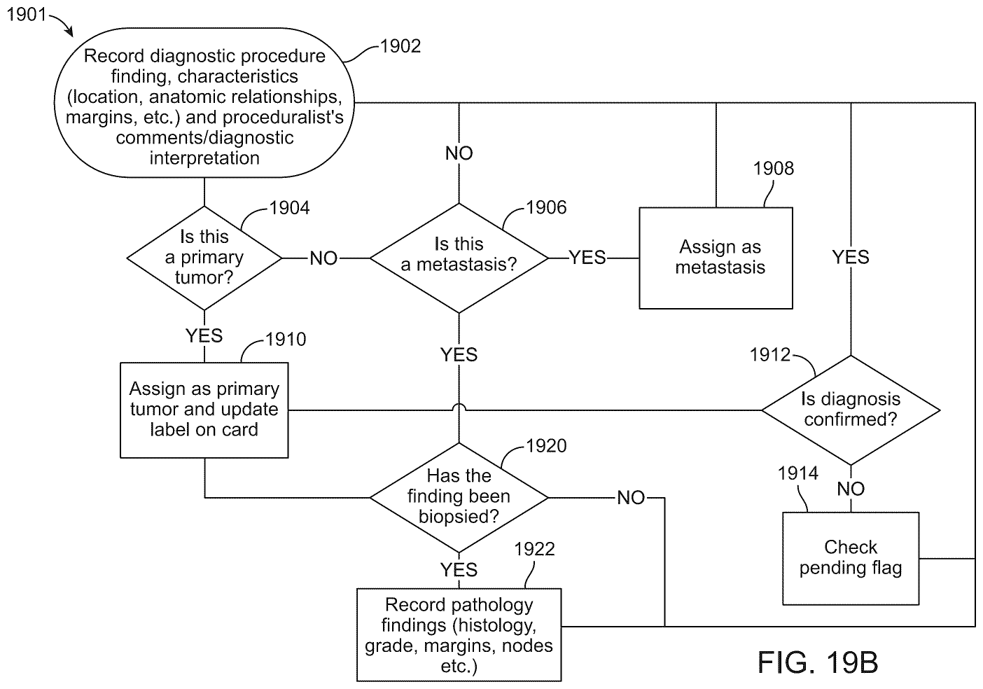

FIG. 19A and FIG. 19B illustrate examples of an oncology workflow that can be implemented by oncology workflow application 222. The goal of the workflow of FIG. 19A and FIG. 19B is to maintain a detailed curated table of relevant radiographic, procedural, and pathologic findings related to the primary tumor and its associated metastatic lesions which is updated through the course of cancer treatment and other facets of the patient journey. The primary tumor and the metastatic lesions are treated as target lesions. The measurements can be captured as structured data, allow judgment about tumor response or progression to be more objectively, and better inform judgements about the patient's clinical status. As findings change, they are recorded in an iterative fashion. FIG. 19A illustrates an example chart 1900 that show the change of lesion size with respect to time, for different target lesions, that can be obtained from the example workflow.

FIG. 19B illustrates a flowchart 1901 of an example of a oncology workflow which allows an oncologist to select a target lesion for monitoring and for response evaluation. Referring to FIG. 19B, in step 1902, diagnostic procedure findings, characteristics of the finding, and proceduralists' comments/diagnostics interpretation of the finding, are recorded based on data received from medical data processing system 200. In step 1904, a determination is made about whether the finding (e.g., a lesion, etc.) indicates a primary tumor. If the lesion is neither a primary tumor (in step 1904) nor a metastasis (in step 1906), the iteration can end, and step 1902 is then repeated at later time to record new diagnostic procedure findings, characteristics of the finding, and proceduralists' comments/diagnostics interpretation of the finding. If the lesion is a metastasis (in step 1906), the finding can be assigned as metastasis in data entry interface 300 in step 1908. The assignment of metastasis can be performed in patient summary page 311 as shown in FIG. 3F, and the patient's oncology data can be updated, the iteration can then end, and step 1902 can be repeated.

On the other hand, if the lesion is a primary tumor (in step 1904), the lesion is assigned as primary tumor via data entry interface 300, as shown in operation 340 of FIG. 3D, in step 1910. If the diagnosis is confirmed (in step 1912), the patient's oncology data can be updated. If the diagnosis is not confirmed (in step 1912), the “pending diagnosis” flag 321 of FIG. 3B can remain asserted, in step 1914. In both cases, the iteration can end, and step 1902 can be repeated. Moreover, from step 1910, a determination can be made about whether a biopsy has been performed, in step 1920. If the finding has been biopsied (in step 1920), pathology findings can be recorded as part of the structured data of the patient, in step 1922. Step 1902 is then repeated at later time to record new diagnostic procedure findings, characteristics of the finding, and proceduralists' comments/diagnostics interpretation of the finding.

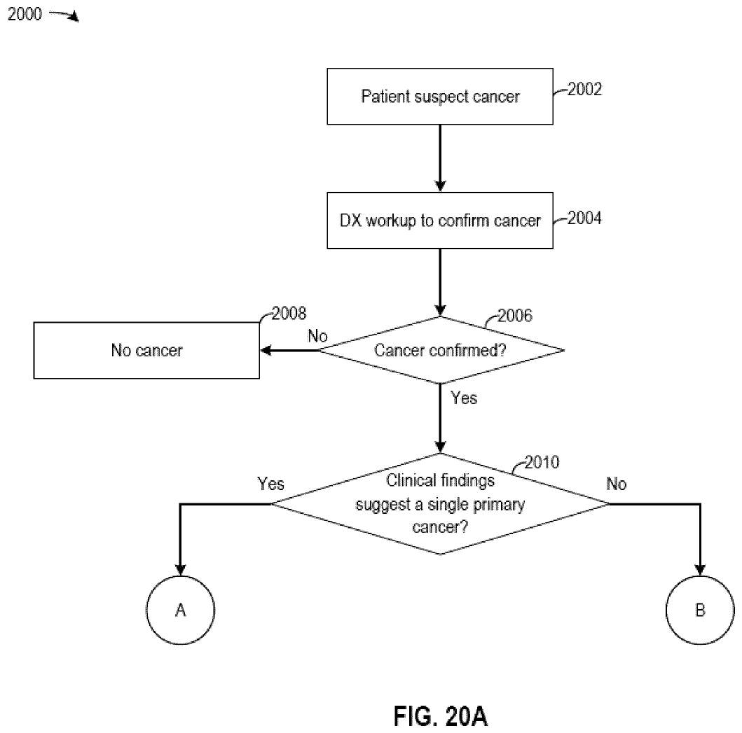

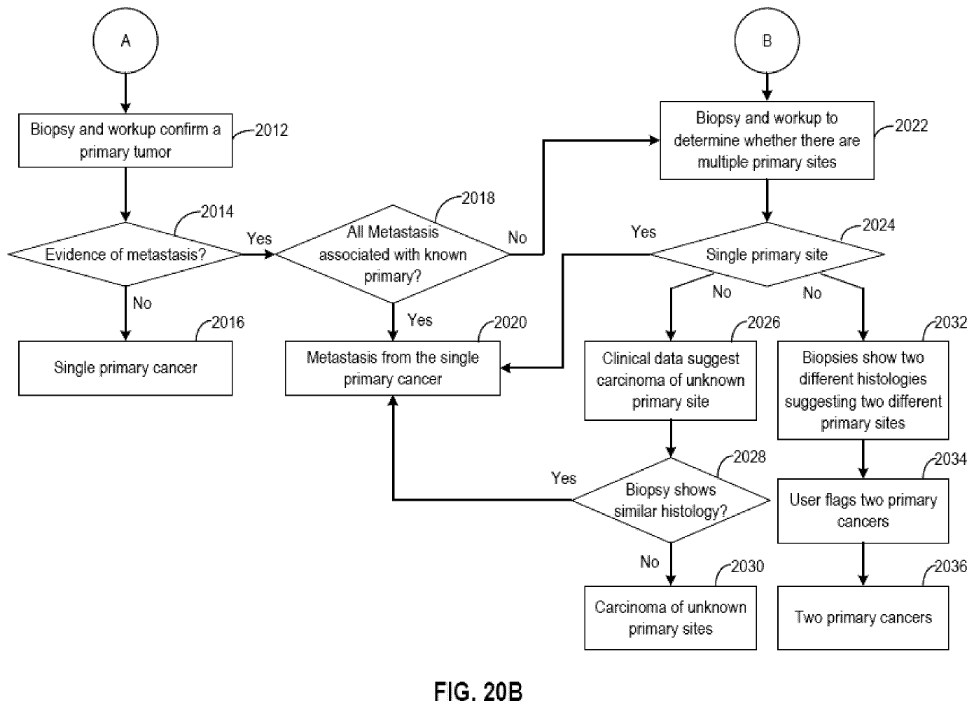

FIG. 20A and FIG. 20B illustrate a flowchart 2000 of another example oncology workflow. The oncology workflow of flowchart 2000 enables oncologists (and their delegates) to longitudinally manage cancer patients from suspicion of cancer through treatment and follow-up by leveraging the full context of patient information. Referring to FIG. 20A, data collection module 230 can collect medical data, via portal 220, of a patient who suspects cancer, in step 2002. In step 2004, an oncologist can analyze the data to confirm whether the patient has cancer. If no cancer is confirmed (in steps 2006 and 2008), the oncology workflow can end. But if cancer is confirmed, a determination is made about whether clinical findings suggest a single primary cancer, in step 2010.

Referring to FIG. 20B, if clinical findings suggest a single primary cancer (in step 2010), biopsy and workup data can be analyzed to confirm a primary tumor, in step 2012. If there is no evidence of metastasis (in step 2014), it can be concluded that the patient has single primary cancer, in step 2016. On the other hand, if there is evidence of metastasis (in step 2014), and all metastasis is associated with known primary (in step 2018), it can concluded that there is metastasis from the single primary cancer, in step 2020.

If clinical findings do not suggest a single primary cancer (in step 2010), or that the metastasis is not associated with known primary (in step 2018), biopsy and workup data can be analyzed to determine whether there are multiple primary sites, in step 2022. If the biopsy and workup data of step 2022 confirm there is only a single primary site, it can be determined that the metastasis is from the single primary cancer, in step 2020. But if the biopsy and workup data of step 2022 cannot confirm there is only a single primary site, the workflow can proceed with different routes. For example, if the clinical data suggest carcinoma of unknown primary site (in step 2026), and biopsy shows similar histology (step 2028), it can be determined that the metastasis is from the single primary cancer, in step 2020. But if the clinical data suggest carcinoma of unknown primary site (in step 2026), and biopsy shows different histologies (in step 2028), it can be determined that the patient has carcinoma of unknown primary sites, in step 2030. Moreover, returning back to step 2024, if biopsies show two histologies suggesting two different primary sites (step 2032), and that user flags two primary cancers (e.g., via assigning a mass to a primary cancer site, as in FIG. 3D and FIG. 3E) in step 2034, it can be determined that the patient has two primary cancers, in step 736. Certain diagnostic results (e.g., finding of a tumor mass) can be associated with the second primary tumor, as in FIG. 3F.

G. Method of Processing Medical Data to Facilitate a Clinical Decision

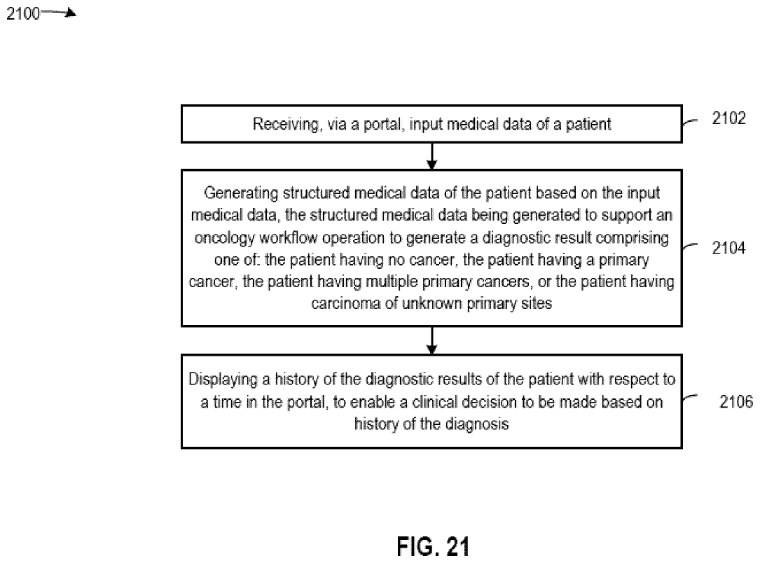

FIG. 21 illustrates a method 2100 of processing medical data to facilitate a clinical decision. Method 2100 can be performed by, for example, medical data processing system 200 of FIG. 2.

In step 2102, medical data processing system 200 receives, via a portal (e.g., portal 220), input medical data of a patient. The patient data can originate from various data sources (at one or more healthcare institutions) including, for example, an EMR (electronic medical record) system, a PACS (picture archiving and communication system), a Digital Pathology (DP) system, a LIS (laboratory information system) including genomic data, RIS (radiology information system), patient reported outcomes, wearable and/or digital technologies, social media etc.

In some examples, the portal can provide a data entry interface, which includes various fields to receive the input medical data, and structured medical data can be generated based on the mapping between the fields and the data. The structured medical data can include various information related to the diagnosis of a tumor, such as tumor site, staging, pathology information (e.g., biopsy results), diagnostic procedures, and biomarkers of both the primary tumor as well as additional tumor sites (e.g., due to metastasis from the primary tumor). The portal can display the structured data in the form of a patient summary. The portal can also organize the display of the structured data into pages, with each page being associated with a particular primary tumor site and including the fields of information of the associated primary tumor site and can be accessed by a tab. Based on detecting the user's input of certain fields in the page of a first primary tumor (e.g., designation of an additional tumor site as a new primary tumor), the portal can create an additional page for a second primary tumor, and populate the fields of the newly-created page for the second primary tumor based on the addition tumor site information input into the page of the first primary tumor. In some examples, the portal processor also allows a user to select an additional tumor mass found during a diagnostic procedure of the primary tumor and associate the mass with the second primary tumor to represent the case of metastasis. Based on detecting the association, medical data processor can transfer all the diagnostic results of the additional tumor from the first primary tumor page to the newly-created page for the second primary tumor.

In some examples, the portal also allows a user to import a document file (e.g., a pathology report, a doctor note, etc.) from the aforementioned data sources. The medical data abstraction module can then extract various structured medical data from the document file. The structured medical data can be extracted based on performing, for example, a natural language processing (NLP) operation, a rule-based extraction operation, etc., on the texts included in the document file. The medical data abstraction module also allows manual extraction of structured medical data from the document file via the portal. The portal can then display the extracted medical data in addition to the document file.

For example, the portal can overlay texts of the file with highlight markings. The portal can also display text boxes including the medical data extracted from the texts over the highlighted texts. In addition, the structured medical data can also be extracted from various metadata of the document file, such as date of the file, category of the document file (e.g., a pathology report versus a clinician's note), the clinician who authored/signed off the document file, and a procedure type associated with the content of the document file (e.g., biopsy, imaging, or other diagnosis steps). The portal can then populate various fields of a page based on the extracted data. Various enrichment operations can also be performed on the extracted data to improve the quality of the extracted medical data. One enrichment operation can include a normalization operation to normalize various numerical values (e.g., weight, tumor size, etc.) included in the extracted medical data to a standardized unit, to correct for a data error, or to replace a non-standard terminology provided by a patient with a standardized terminology based on various medical standards/protocols, such as International Classification of Diseases (ICD) and Systematized Nomenclature of Medicine (SNOMED). The enriched extracted medical data can then be stored in a unified patient database as part of the structured medical data (e.g., structured oncology data) for the patient.

In step 2104, structured medical data is generated based on the input medical data. The structured medical data are generated to support an oncology workflow operation to generate a diagnostic result comprising one of: the patient having no cancer, the patient having a primary cancer, the patient having multiple primary cancers, or the patient having carcinoma of unknown primary sites. Examples of the oncology workflow are described in FIG. 19A-FIG. 20B. The oncology workflow can also perform a diagnosis operation based on the structured medical data. In one example, the diagnosis operation can be performed to confirm whether a biopsy result is for the same primary tumor or is for a different tumor, and to track the size of the primary tumor for evaluating the tumor's response to particular treatment. In another example, the diagnosis operation can be performed to determine whether the patient has a single primary tumor site, multiple primary tumor sites, or unknown primary sites. The results of the diagnosis operation can then be recorded and/or displayed with respect to time in the portal as part of the medical journey of the patient, to enable an oncologist or his/her delegates to longitudinally manage cancer patients from suspicion of cancer through treatment and follow-up. The diagnosis results can also be used to support other medical applications, such as a quality of care evaluation tool to evaluate a quality of care administered to a patient, a medical research tool to determine a correlation between various information of the patient (e.g., demographic information) and tumor information (e.g., prognosis or expected survival) of the patient, etc.

In step 2106, the portal can display a history of the diagnostic results of the patient with respect to a time, to enable a clinical decision to be made based on history of the diagnosis. For example, the portal can display a timeline representing the patient's medical journey, as shown in FIGS. 9A-9E, which can include a history of the primary tumor size, a history of other diagnostic results, etc. This allows the clinician to make a clinical decision about, for example, a treatment to be administered to the patient.

### V. Example Computer System

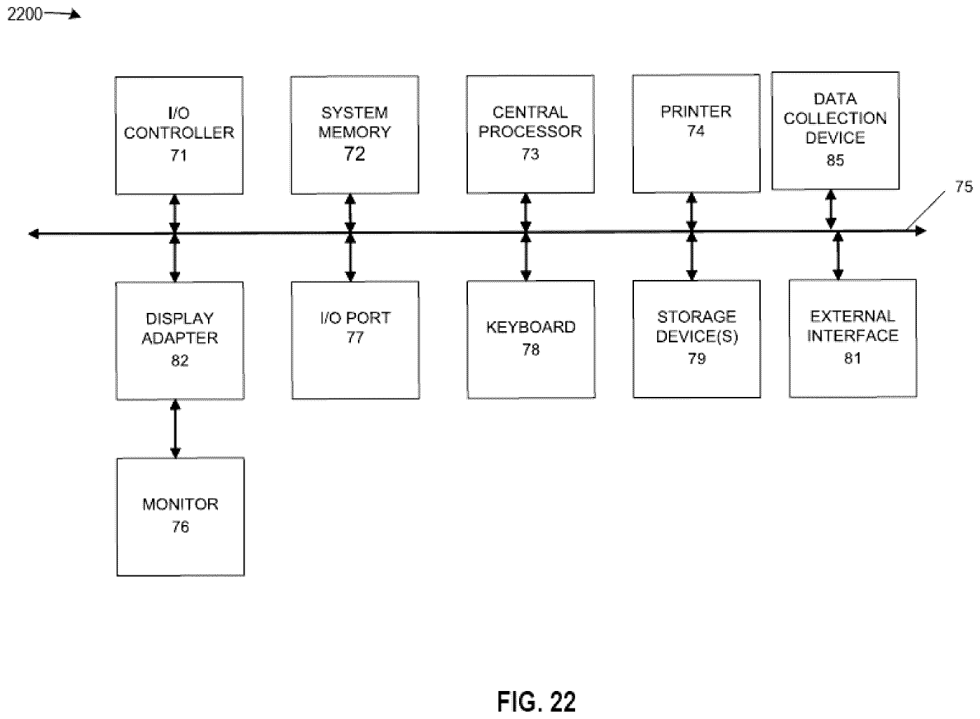

Any of the computer systems mentioned herein may utilize any suitable number of subsystems. Examples of such subsystems are shown in FIG. 22 in the computer system 2200. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 22 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect the computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A method for managing medical data comprising performing by a server computer:
creating a patient record for a patient in a unified patient database, the patient record comprising an identifier of the patient and one or more data objects related to medical data associated with the patient, the unified patient database including data from a plurality of sources;
retrieving, from an external database, a medical record for the patient;
receiving identification of a primary cancer associated with the medical record via a Graphical User Interface (GUI);
in response to receiving the identification of the primary cancer, creating a primary cancer object in the patient record, the primary cancer object having a field including the primary cancer;
storing the medical record linked to the primary cancer object in the patient record in the unified patient database;
receiving, via user input to the GUI, medical data for the patient;
determining that the medical data for the patient is associated with the primary cancer; and
storing the medical data for the patient linked to the primary cancer object in the patient record in the unified patient database.

2. The method of claim 1, wherein:
the medical record for the patient is in a first format comprising a set of data elements correlated to corresponding data types; and
receiving the identification of the primary cancer comprises:
identifying the primary cancer by analyzing the data elements and the data types;
displaying the GUI comprising a prompt for a user to confirm the primary cancer identification; and
receiving user confirmation of the primary cancer identification via the GUI.

3. The method of claim 2, wherein the medical record is a first medical record, the method further comprising:
receiving a second medical record for the patient, wherein the second medical record is in a second format comprising unstructured data;
identifying, from the unstructured data, a data element associated with the primary cancer;
analyzing the unstructured data to assign the data element to a data type; and
based on the assigned data type and the identifying the data element is associated with the primary cancer, storing the data element linked to the primary cancer object in the patient record in the unified patient database.

4. The method of claim 1, wherein receiving the identification of the primary cancer associated with the medical record comprises:
displaying, via the GUI, the medical record and a menu configured to receive user input selecting one or more primary cancers; and
receiving, via the GUI, user input selecting the primary cancer.

5. The method of claim 4, further comprising:
storing the medical record in the patient record; and
parsing the medical record to determine that the patient record is not associated with a particular primary cancer,
wherein displaying the medical record and the menu is responsive to determining that the patient record is not associated with a particular primary cancer.

6. The method of claim 1, wherein:
the medical record comprises unstructured data; and
the method further comprises:
applying a first machine learning model to identify text in the medical record; and
applying a second machine learning model to correlate a portion of the identified text with a corresponding field,
wherein storing the medical record further comprises storing the identified text to the unified patient database in association with the field.

7. The method of claim 6, wherein:
the first machine learning model comprises an Optical Character Recognition (OCR) model; and
the second machine learning model comprises a Natural Language Processing (NLP) model.

8. The method of claim 1, further comprising:
retrieving, from the unified patient database, at least a subset of the medical data for the patient; and
causing display, via a user interface, of the at least the subset of the medical data for the patient for performing clinical decision making.

9. A method for managing a unified patient database comprising performing by a server computer:
storing, to the unified patient database, a patient record comprising a network of interconnected data objects, the unified patient database including data from a plurality of sources;
storing, to the patient record in the unified patient database, a first data object corresponding to a data element for a tumor mass of a primary cancer, the first data object including an attribute specifying a site of the tumor mass;
receiving, from a diagnostic computer, diagnosis information corresponding to the primary cancer;
analyzing the diagnosis information to identify a correlation between the diagnosis information and to the tumor mass;
based on identifying the correlation between the diagnosis information and the tumor mass, storing, to the unified patient database, a second data object corresponding to the diagnosis information, the second data object connected to the first data object via the network of interconnected data objects;
receiving, from the diagnostic computer, treatment information corresponding to the primary cancer;
analyzing the treatment information to identify a correlation between the treatment information and to the tumor mass; and
based on identifying the correlation between the treatment information and the tumor mass, storing, to the unified patient database, a third data object corresponding to the treatment information, the third data object connected to the first data object via the network of interconnected data objects.

10. The method of claim 9, further comprising:
retrieving, from the unified patient database, one or more of the attributes specifying the site of the tumor mass, the diagnosis information, and/or the treatment information; and
causing display, via a user interface, of one or more of the attribute specifying the site of the tumor mass, the diagnosis information, and/or the treatment information for clinical decision making.

11. The method of claim 9, further comprising:
receiving, from the diagnostic computer, patient history data;
analyzing the patient history data to identify a correlation between the patient history data and the tumor mass; and
based on identifying the correlation between the patient history data and the tumor mass, storing, to the unified patient database, a fourth data object corresponding to the patient history data, the fourth data object connected to the first data object via the network of interconnected data objects.

12. The method of claim 9, further comprising:
receiving, from the diagnostic computer, tumor mass information corresponding to a tumor mass at a metastasis site of the primary cancer;
analyzing the tumor mass information to identify a correlation between the diagnosis information and the tumor mass; and
based on receiving the tumor mass information and identifying the first data object, storing, to the unified patient database, a fifth data object corresponding to the tumor mass information connected to the first data object via the network of interconnected data objects.

13. The method of claim 9, wherein the second data object includes one or more attributes selected from: a stage of the primary cancer, a biomarker, and a tumor size.

14. The method of claim 9, further comprising:
identifying, from the unified patient database, a data element and a data type associated with the patient; and
transmitting, to an external system, the data element and the data type in structured form.

15. The method of claim 9, further comprising, upon generating each of the first data object and the second data object, generating a first timestamp stored in association with the first data object indicating a time of creation of the first data object and a second timestamp stored in association with the second data object indicating the time of creation of the second data object.

16. The method of claim 9, further comprising updating the unified patient database by:
importing medical data from an external database;
parsing the imported medical data to identify a particular data element associated with the patient and the primary cancer; and
storing the particular data element to a sixth data object in association with the first data object.

17. A method of processing medical data to facilitate a clinical decision, comprising:
receiving, via a portal, input medical data of a patient associated with a plurality of data categories, the plurality of data categories being associated with an oncology workflow operation;
generating structured medical data of the patient based on the input medical data, the structured medical data being generated to support the oncology workflow operation to generate a diagnostic result comprising one of: the patient having no cancer, the patient having a primary cancer, the patient having multiple primary cancers, or the patient having a carcinoma of unknown primary sites; and
displaying, via the portal, the structured medical data and a history of the diagnostic results of the patient with respect to a time in the portal, to enable a clinical decision to be made based on the history of the diagnosis results.

18. The method of claim 17, wherein the portal comprises a data entry interface to receive the input medical data, and to map the input medical data into fields to generate the structured medical data; and
wherein the data entry interface organizes the structured medical data into one or more pages, each of the one or more pages being associated with a particular primary tumor site.

19. The method of claim 18, further comprising:
receiving, via the data entry interface, a first indication that a first subset of the medical data entered into a first page of the data entry interface associated with a first primary tumor site belongs to a second primary tumor site; and
based on the first indication:
creating a second page for the second primary tumor site; and
populating the second page with the first subset of the medical data.

20. The method of claim 19, further comprising:
receiving, via the data entry interface, a second indication that a second subset of the medical data entered into the first page is related to a metastasis of the second primary tumor site; and
based on the second indication, populating the second page with the second subset of the medical data.
